(19) United States
(12) Patent Application Publication
(10) Pub. No.: US 2026/0063623 A1
(43) Pub. Date: Mar. 5, 2026

(54) METHODS OF ASSESSING METABOLIC FLUX

(71) Applicant: The Regents of the University of Michigan, Ann Arbor, MI (US)

(72) Inventors: Andrew Scott, Ann Arbor, MI (US); Abhinav Achreja, Ann Arbor, MI (US); Anjali Mittal, Ann Arbor, MI (US); Deepak Nagrath, Ann Arbor, MI (US); Costas A. Lyssiotis, Ann Arbor, MI (US); Daniel Wahl, Ann Arbor, MI (US); Baharan Meghdadi, Ann Arbor, MI (US)

(21) Appl. No.: 19/120,865

(22) PCT Filed: Oct. 13, 2023
(86) PCT No.: PCT/US2023/076858

§ 371 Date: Apr. 14, 2025

Related U.S. Application Data
(60) Provisional application No. 63/416,146, filed on Oct. 14, 2022.

(51) Int. Cl. G01N 33/50; G16C 20/70

(52) U.S. Cl. CPC G01N 33/5088; G01N 33/5038; G16C 20/70

(57) ABSTRACT

The present disclosure relates to methods for assessing metabolic flux. In some aspects, the disclosure relates to methods for estimating absolute metabolic flux for a pathway of interest based upon a level of one or more metabolites and/or isotopologues thereof at a single point in time following administration of a tracer to a subject. In some embodiments, the methods described herein are performed or generated using artificial intelligence/machine-learning (AI/ML) models.

### PRIORITY STATEMENT

This application claims priority to U.S. Provisional Application No. 63/416,146, filed Oct. 14, 2022, the entire contents of which are incorporated herein by reference.

### FIELD

The present disclosure relates to methods for assessing metabolic flux. In some aspects, provided herein is a method for estimating absolute metabolic flux for a pathway of interest based upon a level of one or more metabolites and/or isotopologues thereof at a single point in time following administration of a tracer to a subject. In some embodiments, the methods described herein are performed or generated using artificial intelligence/machine-learning (AI/ML) models.

### BACKGROUND

Knowledge of metabolic rates in a metabolic network provides insight into the regulation of metabolism and the contribution of metabolic alterations to pathology. However, several challenges exist in in-vivo flux quantification: (1) metabolite uptake and secretion fluxes (i.e. exchange fluxes) cannot be measured experimentally, (2) In-vivo experiments are resource extensive and can only be performed for a few hours, which is not enough to achieve isotopic steady state, and (3) Inter-organ metabolism leads to secondary enrichment in circulating metabolites. Accordingly, improved methods for adequately assessing metabolic flux are needed.

### SUMMARY

In some aspects, provided herein are methods of estimating absolute metabolic flux for a pathway of interest. In some embodiments, methods of estimating absolute metabolic flux for a metabolic pathway of interest comprise administering a tracer to subject, collecting a sample from the subject, and generating an estimate of absolute metabolic flux for a pathway of interest. In some embodiments, the sample is a tissue sample. In some embodiments, the estimate of absolute metabolic flux for the pathway of interest is generated based upon a level of one or more metabolites and/or isotopologues thereof in the sample at a single point in time following administration of the tracer to the subject.

In some embodiments, the pathway of interest is a purine synthesis pathway. In some embodiments, the pathway of interest is a purine synthesis pathway and the one or more metabolites and/or isotopologues thereof comprise one or more of Ribose-5-Phosphate (R5P), Glycine (GLY), Carbon Dioxide (CO2), 5-Methyltetrahydrofolate (C-THF), Inosine Monophosphate (IMP), Inosine, Hypoxanthine, Guanine, Guanosine Monophosphate (GMP), Guanosine Diphosphate (GDP), Guanosine, Adenosine Monophosphate (AMP), and Adenosine. In some embodiments, the method further comprises determining a contributing fraction of de novo GMP synthesis and/or de novo IMP synthesis in the purine synthesis pathway.

In some embodiments, the pathway of interest is a pyrimidine synthesis pathway. In some embodiments, the pathway of interest is a pyrimidine synthesis pathway and the one or more metabolites and/or isotopologues thereof comprise one or more of Ribose-5-Phosphate (R5P), Aspartate (ASP), Carbon Dioxide (CO2), Uridine, and Uridine Monophosphate (UMP).

In some embodiments, the pathway of interest is a serine synthesis pathway (e.g. a de novo serine synthesis pathway). In some embodiments, the pathway of interest is a serine synthesis pathway and the one or more metabolites and/or isotopologues thereof comprise one or more of 3-phosphoglycerate (3PG), glycine (gly), and 5,10-methylene-THF (Me-THF).

In some embodiments, subject has cancer, and the tissue sample comprises a tumor tissue sample. In some embodiments, the estimate of absolute metabolic flux for the pathway of interest is generated based upon a level of one or more metabolites and/or isotopologues thereof in the sample at a single point in time following administration of radiation therapy to the subject.

In some embodiments, estimate of absolute metabolic flux for the pathway of interest is generated using an artificial intelligence/machine learning (AI/ML) model.

In some embodiments, the tracer comprises a moiety labeled with ¹³C, ¹⁵N, ¹⁸O, or ²D. In some embodiments, the moiety comprises glucose, methionine, serine, glutamine, lactate, acetate, hypoxanthine, or uridine.

In some aspects, provided herein are methods of selecting therapy for a subject in need thereof. In some embodiments, methods of selecting therapy for a subject in need thereof comprise generating an estimate of absolute metabolic flux for a pathway of interest using the methods described herein, and selecting a therapy for a subject in need thereof based upon the estimate of absolute metabolic flux for the pathway of interest. In some embodiments, the subject has cancer, and wherein the pathway of interest is a cancer-related pathway. In some embodiments, the method further comprises the step of administering the selected therapy to the subject. For example, the method may further comprise administering a selected anti-cancer therapy to a subject based upon the estimate of absolute metabolic flux for a pathway of interest in the sample obtained from the subject.

In some aspects, provided herein are machine-learning methods for estimating absolute activity of one or more metabolic fluxes. In some embodiments, machine-learning methods for estimating the absolute activity of one or more metabolic fluxes comprise obtaining a plurality of measurements from a sample collected from a subject, and applying an artificial intelligence/machine learning (AM/ML) model to the measurements to generate an estimate of absolute activity of one or more metabolic fluxes. In some embodiments, each measurement is a level of a metabolite and/or an isotopologue thereof in a metabolic pathway of interest at a single point in time.

In some embodiments, the sample comprises a tissue sample. In some embodiments, the subject is diagnosed with or at risk of having cancer. In some embodiments, the subject is diagnosed with or at risk of having cancer and the tissue sample is a tumor tissue sample. In some embodiments, the plurality of measurements are obtained from the sample after administering radiation therapy to the subject.

In some embodiments, the plurality of measurements are obtained from the sample after administering a tracer to the subject. In some embodiments, the tracer comprises a moiety labeled with ¹³C, ¹⁵N, ¹⁸O, or ²D. In some embodiments, the moiety comprises glucose, methionine, serine, glutamine, lactate, acetate, hypoxanthine, or uridine.

In some aspects, provided herein are methods selecting a therapy for a subject in need thereof, comprising estimating absolute activity of one or more metabolic fluxes by the method described herein, and selecting therapy for a subject in need thereof based upon the estimated absolute activity of the one or more metabolic fluxes. In some embodiments, the subject has cancer. In some embodiments, the method further comprises the step of administering the selected therapy to the subject. For example, the method may further comprise administering a selected anti-cancer therapy to the subject based upon the estimated activity of the one or more metabolic fluxes.

In some aspects, provided herein are methods of selecting a therapy for a subject having cancer, comprising determining a contributing fraction of de novo GMP synthesis and/or de novo IMP synthesis in a purine synthesis pathway, and selecting a inosine monophosphate dehydrogenase (IMPDH) inhibitor for the subject when the contributing fraction of de novo GMP synthesis and/or de novo IMP synthesis is increased relative to a threshold value.

In some aspects, provided herein are methods of selecting a therapy for a subject having cancer, comprising determining absolute activity of a serine synthesis pathway (e.g. a de novo serine synthesis pathway) and selecting dietary serine restriction for the subject when the absolute activity of a serine synthesis pathway is decreased relative to a threshold value.

In some embodiments, the subject has a brain cancer. In some embodiments, the subject has a glioblastoma.

### DESCRIPTION OF THE DRAWINGS

FIG. 1. Data acquisition methods. (FIG. 1A) Isotope labeling of metabolites from tissues of orthotopic GBM bearing mice at multiple time points under different treatment conditions are used to generate models. (FIG. 1B) Patients are infused with uniformly labeled ¹³C-glucose during brain tumor resection, and plasma and resected tissues are collected for label assessment of metabolites. (FIG. 1C) Plasma enrichment of isotopic glucose is measured by LC-MS. (FIG. 1D) Relative guanylate labeling in human tumor compared to adjacent cortex tissue.

FIG. 2. Reaction network of quantified purine pathway fluxes. AMP: adenosine monophosphate; C-THF: formyl tetrahydrofolate; CO2: carbon dioxide; IMP: inosine monophosphate; GDP: guanosine diphosphate; GMP: guanosine monophosphate; R5P: Ribose-5-Phosphate.

FIG. 3. Estimated flux values for the INST-MFA model. The fluxes were estimated for four conditions: GBM without radiation treatment, GBM after radiation treatment, adjacent normal cortex tissue without radiation treatment, and the normal cortex tissue after radiation treatment.

FIG. 4A-4D. MFA reveals higher flux through IMPDH after radiation therapy (RT). (FIG. 4A) De novo GMP synthesis in treatment-naïve mice model. (FIG. 4B) Dynamic profile of de novo IMP (Inosine Monophosphate) synthesis after RT. IMP is the precursor for de novo GMP synthesis after RT. (FIG. 4C) Dynamic profile of de novo GMP synthesis. (FIG. 4D) Fraction of total GMP flux synthesized de novo via IMPDH after RT. RT: radiation treatment.

FIG. 5 shows an exemplary architecture for a convolutional neural network (CNN model) described herein.

FIGS. 6A-6H show robust [U¹³C]-glucose uptake and utilization in mouse models and patients with brain cancer. (A) Schema of [U¹³C]-glucose infusions into patients and mouse models of brain cancer. (B) Example of MRI-defined tissue acquisition. (C) Clinical and molecular characteristics of patients studied with stable isotope tracing. (D) Time course of M+6 arterial glucose enrichment in patients (top) and mice (bottom) undergoing [U¹³C]-glucose infusions. For mice, error bars indicate SD from 2-10 mice. (E) Schema of glucose carbon (red circles) redistribution into glycolytic intermediates. Scrambling can occur via recombination with unlabeled intermediates in the pentose phosphate cycle (E4P, S7P, R5P, GAP). (F) Mean enrichment of glycolytic intermediates in GBM (gold) and cortex (blue) tissue isolated from orthotopic GBM38-bearing mice infused with [U¹³C]-glucose (n=7 mice, error bars indicate SD). (G). Normalized (to plasma M+6 glucose on a per-patient basis) enrichment of glycolytic intermediates in cortex (blue), non-enhancing tumor (grey) and enhancing tumor (orange) from 8 patients infused with [U¹³C]-glucose (n=7-8 samples per group, error bars indicate SD). (H) H&E staining of GBM38 PDX grown orthotopically (left) and MALDI image showing ¹³C enrichment of lactate with tissue maximum set at 100%. * p<0.05. Metabolite abbreviations as follows: FBP (Fructose Bisphosphate), GAP (Glyceraldehyde phosphate), DHAP (dihydroxyacetone phosphate), PG (phosphoglycerate), PEP (phosphoenolpyruvate), Pyr (pyruvate), Lac (lactate), G6P (glucose 6-phosphate), F6P (Fructose 6-phosphate), R5P (ribose 5 phosphate), E4P (erythrose 4-phosphate), S7P (sedoheptulose 7-phosphate), 3PG (3-phosphoglycerate).

FIGS. 7A-7I show decreased TCA cycle and neurotransmitter labeling in brain cancer. (A) Schema of ¹³C labeling in TCA cycle intermediates and neurotransmitters arising from M+3 pyruvate. Red circles indicate entry through pyruvate dehydrogenase, and orange indicates entry through pyruvate carboxylase. Blue circles indicate labeling patterns possible on second TCA cycle turn. (B) Mean enrichment of TCA cycle intermediates and aspartate in GBM (gold) and cortex (blue) tissue isolated from orthotopic GBM38-bearing mice infused with [U¹³C].glucose (n=7 mice, error bars indicate SD). (C) Mean enrichment of glutamine and glutamate in GBM (gold) and cortex (blue) tissue isolated from orthotopic GBM38-bearing mice infused with [U¹³C]-glucose (n=7 mice, error bars indicate SD). (D) Normalized (to plasma M+6 glucose on a per-patient basis) enrichment of TCA cycle intermediates and aspartate in cortex (blue), non-enhancing tumor (grey) and enhancing tumor (orange) from 8 patients infused with [U¹³C]-glucose (n=7-8 samples per group and error bars indicate SD). (E) Normalized (to plasma M+6 glucose on a per-patient basis) enrichment of glutamate and glutamine in cortex (blue), non-enhancing tumor (grey) and enhancing tumor (orange) from 8 patients infused with [U¹³C]-glucose (n=7-8 samples per group, error bars indicate SD). Mean enrichment (F) and normalized mean enrichment (G) of GABA in mouse (F) and human (G) samples. (H) Malate normalized mean enrichment by MALDI. Color bar tissue maximum normalized to 100%. (G) Malate % enrichment in representative human cortex (left) non-enhancing tumor (middle) and enhancing tumor (right). Color bar maximum set at true enrichment. Abbreviations: aKG (alpha-ketoglutarate), NME (normalized mean enrichment). * p<0.05, ** p<0.01.

FIGS. 8A-9H show increased glucose-driven nucleotide synthesis in brain cancer. (A) Schema of purine synthetic pathways. Green and yellow circles indicate glycine- and folate-derived carbons, respectively. Blue circles indicate ribose 5-phosphate (R5P)-derived carbons (different R5P labeling patterns are shown to indicate potential pentose phosphate cycle scrambling). (B) Mean enrichment of purines in GBM (gold) and cortex (blue) tissue isolated from orthotopic GBM38-bearing mice infused with [U¹³C]-glucose (n=7 mice, error bars indicate SD). (C) Normalized (to plasma M+6 glucose on a per-patient basis) enrichment of purines in cortex (blue), non-enhancing tumor (grey) and enhancing tumor (orange) from 8 patients infused with [U¹³C]-glucose (n=7-8 samples per group, error bars indicate SD). (D) Schema of pyrimidine synthetic pathways. Purple indicates aspartate-derived carbons, blue indicates R5P-derived carbons, and green indicates folate-derived carbons. (E) Mean enrichment of pyrimidines in GBM (gold) and cortex (blue) tissue isolated from orthotopic GBM38-bearing mice infused with [U¹³C₆]-glucose (n=7 mice, error bars indicate SD). (F) Normalized (to plasma M+6 glucose on a per-patient basis) enrichment of pyrimidines in cortex (blue), non-enhancing tumor (grey) and enhancing tumor (orange) from 8 patients infused with [U¹³C]-glucose (n=7-8 samples per group, error bars indicate SD). (G) Normalized (to plasma M+6 glucose on a per-patient basis) enrichment of NAD and NADH in cortex (blue), non-enhancing tumor (grey) and enhancing tumor (orange) from 8 patients infused with [U¹³C]-glucose (n=7-8 samples per group, error bars indicate SD). (H) AMP M+5 signal intensity by MALDI in mouse GBM and cortex. Color bar tissue maximum normalized to 100%. Abbreviations: Me-THF (N10-formyltetrahydrofolate). AMP (adenosine monophosphate), IMP (inosine monophosphate), GMP (guanosine monophosphate), GDP (guanosine diphosphate), ADP (adenosine diphosphate), 5,10-MeTHF (5,10-methylenetetrahydrofolate), UMP (uridine monophosphate), CMP (cytidine monophosphate), dTDP (deoxythymidine diphosphate), NAD (oxidized nicotinamide adenine dinucleotide), NADH (reduced nicotinamide adenine dinucleotide). * p<0.05, ** p<0.01, *** p<0.001.

FIGS. 9A-9F show quantification of metabolic fluxes in GBM and cortex. (A) Schema. [U¹³C]-glucose was infused into GBM38 PDX-bearing mice. Mice were serially euthanized at varying timepoints (0, 30, 120 and 240 min), and GBM and cortex was harvested to measure metabolite enrichment and estimate metabolic fluxes. (B-C) Absolute fluxes of purine (B) and pyrimidine (C) synthetic reactions in cortex (left) and GBM (right). * indicates p<0.05 comparing GBM and cortex. (D-E) Percent abundance of individual malate isotopologues in cortex (D) or GBM (E) Error bars indicate SD of n=3-9 datapoints from n=1-3 mice at each timepoint. (F) Percent change in indicated malate isotopologues from 120 to 240 min. Error bars are propagated from uncertainty in 120 and 240 min datapoints in (D) and (E).

FIGS. 10A-10I show dynamic metabolic response to radiation in GBM and cortex. (A) Schema of dynamic metabolic flux analysis pipeline. (B) Overview of purine synthesis pathways. (C-I) dynamic metabolic fluxes following RT in GBM (red) and cortex (grey). GBM38 PDX-bearing mice were treated with cranial RT (8Gy) at t=0. Mice were serially euthanized at varying timepoints after RT (0, 30, 60, 120 and 240 min), and GBM and cortex were harvested to estimate metabolic fluxes (n=1-3 mice per group with 1-3 samples/mouse). Solid lines indicate estimated fluxes and shaded regions indicate 95% confidence intervals. Abbreviations: Me-THF (again, I think this is N¹⁰ formyl-tetrahydrofolate), R5P (ribose 5-phosphate), IMP (inosine monophosphate), AMP (adenosine monophosphate).

FIGS. 11A-11H show preferential reliance on environmental serine in brain cancer. (A) Mean ¹³C enrichment of serine in cortex (n=7) or orthotopic GBM38 PDXs (n=7). (B). Normalized (to plasma glucose enrichment) mean enrichment of ¹³C in serine in human samples (n=7-8 per group). (C) Percent serine that contains one (M+1) or three (M+3) tracer-derived ¹³C atoms. (D) Serine isotopologue distributions of tissues from 8 human brain cancer patients. Error bars indicate SD of 3 technical replicates per patient. (E) Isotopologue distributions of plasma serine and tissue phosphoglycerate (PG) in PDXs. (F) Isotopologue distributions of plasma serine and tissue PG in human tissues (PG normalized to plasma glucose enrichment). (G) Metabolic flux analysis model to estimate routes of serine synthesis in cortex and brain cancer. (H) Relative (to cortex) reliance on serine uptake compared to glucose-driven de novo serine synthesis. Significance was tested by comparing the 95% confidence intervals from the flux model. * indicates p<0.05 compared to 1.0 (i.e., significantly different than cortex).

FIGS. 12A-12J show dietary serine restriction selectively alters GBM metabolism and slows tumor growth. (A) Representative bioluminescence imaging of GBM38 PDXs grown orthotopically in mice on control (top) or serine/glycine (Ser/Gly)-restricted diet (bottom). (B). Fold-change in luminescence (compared to mean luminescence on day 3) in GBM38 PDX-bearing mice fed a control diet (black) or Ser/Gly-restricted diet (red). Error bars indicate SEM from 9-10 animals per group. (C-G) Mice in panel B were euthanized approximately 4 weeks after implantation, and brains were bisected for analysis by histopathology (C-E) or metabolomics (F-G). (C-D). Representative H&E (C) or Ki-67 immunohistochemistry (D) images from mice on control or Ser/Gly restricted diets. (E) Ki-67 quantification of GBM38 tumors from mice on a control diet (black, n=5) or Ser/Gly-restricted diet (red, n=4). (F) Heatmap of relative metabolite levels (top 65 PLS-DA) in orthotopic GBM38 tumors from mice fed a control (n=4) or Ser/Gly-restricted diet (n=5), with each row representing a separate tumor. (G) Relative serine and phosphoserine levels in GBM38 tumors and cortex from mice fed control or Ser/Gly-restricted diets. Error bars indicate SD. (H-J) Models of cortical metabolic rewiring in brain cancer. (H) Cortex robustly takes up glucose, which it uses to fuel the TCA cycle and the synthesis of neurotransmitters serine, GABA, and glutamate. (I) Brain cancers upregulate the uptake of environmental serine and downregulate the oxidation of glucose in the TCA cycle and glucose-derived neurotransmitter synthesis. Tumors also re-route glucose-derived carbons to synthesize nucleotides and NAD/NADH, used to drive tumor growth. (J) Restriction of dietary serine forces gliomas to re-route glucose carbon towards serine synthesis, which decreases nucleotide and NAD/NADH levels and slows tumor growth. * p<0.05, ** p<0.01.

FIGS. 13A-13D show circulating lactate enrichment and specimen histology. (A). Time course of M+3 lactate in plasma from patients infused with [U¹³C]-glucose. (B). Time course of M+3 lactate in plasma from orthotopic GBM bearing mice (n between 2 and 10 for each time point) infused with [U¹³C]-glucose. Data are shown as average±standard deviation. (C) Representative hematoxylin and eosin stains of tissues resected from our cohort of patients. (D) Percent tumor content in tissues from each patient was defined by a clinical neuropathologist (SV). Data are shown as average±standard deviation, n=8 patients. *p<0.05. **p<0.01. ns, not significant. Abbreviations: NAA, N-acetylaspartate.

FIGS. 14A-14D show altered metabolite abundances in glioma compared to normal cortex. (A) Levels of NAA in cortical tissue and tumor tissue (enhancing and non-enhancing) from human glioma patients undergoing surgical resection. (B-D) Volcano plots of metabolite abundance determined by LC-MS were used to compare fold change in tumor metabolite levels compared to cortical metabolite levels as follows: (B) enhancing tumor compared to cortex in patients, (C) non-enhancing tumor compared to cortex in patients, and (D) GBM compared to cortex in orthotopic GBM bearing mice. **p<0.01. Abbreviations: NAA, N-acetylaspartate; NAAG, N-acetylaspartylglutamate; GMP, guanosine monophosphate.

FIGS. 15A-15B show isotopic enrichment of UDP-glucose in cortex and glioma. (A) UDP-glucose M+6, predominantly derived from M+6 glucose (left), and UDP-glucose fractional enrichment, accounting for all ¹³C isotopologues (right). N=7 mice. (B) Normalized (to plasma m+6 glucose on a per-patient basis) M+6 UDP-glucose enrichment in tissues from glioma patients infused with [U¹³C]-glucose. Data are shown as average±standard deviation. *p<0.05. ns, not significant.

FIGS. 16A-16G show spatially defined isotope labeling in mouse brain and GBM. (A) Hematoxylin and eosin staining of brains from orthotopic GBM bearing mice intraperitoneally injected with either vehicle (as a negative control for panels B-G) or [U¹³C]-glucose. MALDI-MS was used to determine ¹³C enrichment of (B) lactate, (C) aspartate, (D) GABA, (E) glutamate, (F) glutamine, and (G) AMP M+5. Tissue maximum is set to 100%. Abbreviations: GABA, gamma-aminobutyric acid; AMP, adenosine monophosphate.

FIGS. 17A-17C show spatially defined isotope labeling in human cortex and glioma. The indicated tissue types resected from brain cancer patients receiving either [U¹³C]-glucose infusion (patients 1-8) or no infusion (unlabeled 1, 2) were assessed by MALDI-MS for ¹³C isotope labeling of (A) malate, (B) glutamate, and (C) glutamine.

FIGS. 18A-18C show enrichment of ¹³C-labeled metabolites quantified by spatial MALDI-MS. ¹³C labeling of (A) malate, (B) glutamate, and (C) glutamine for all data points from spatial MALDI-MS scans of tissues resected from glioma patients (shown in FIG. 16). Patients 1-8 were infused with [U¹³C]-glucose, and patients I and II received no infusion. Line inside of each box represents median, boxes represent interquartile range, whiskers represent minimum and maximum values, and points outside whiskers indicate outliers.

FIGS. 19A-19B show production of ribose 5-phosphate from glucose in cortex and brain tumors. (A) Fractional enrichment of ribose 5-phosphate in tissues from orthotopic GBM bearing mice (n=7) infused with [U¹³C]-glucose. (B) Normalized (to plasma m+6 glucose on a per-patient basis) enrichment of ribose 5-phosphate in cortex and tumor tissues from glioma patients infused with [U¹³C]-glucose. Data are shown as average±standard deviation. ns, not significant.

FIGS. 20A-20D show glucose-derived production of inosylate and adenylate purine metabolites in human cortex and glioma. Normalized (to plasma m+6 glucose on a per-patient basis) enrichment of (A) IMP, (B) inosine, (C) AMP, and (D) ADP. Data are shown as average±standard deviation. *p<0.05. **p<0.01. ***p<0.001. ****p<0.0001. ns, not significant. Abbreviations: IMP, inosine monophosphate; AMP, adenosine monophosphate; ADP, adenosine diphosphate.

FIGS. 21A-21C show glucose-derived production of guanylate purine metabolites in human cortex and glioma. Normalized (to plasma m+6 glucose on a per-patient basis) enrichment of (A) guanosine, (B) GMP, and (C) GDP. Data are shown as average±standard deviation. *p<0.05. **p<0.01. ***p<0.001. ****p<0.0001. Abbreviations: GMP, guanosine monophosphate; GDP, guanosine diphosphate.

FIGS. 22A-22B show glucose-derived production of pyrimidine metabolites in human cortex and glioma. Normalized (to plasma m+6 glucose on a per-patient basis) enrichment of (A) UMP and (B) dTDP. Data are shown as average±standard deviation. *p<0.05. **p<0.01. ***p<0.001. ****p<0.0001. Abbreviations: UMP, uridine monophosphate; dTDP, deoxythymidine diphosphate.

FIGS. 23A-23B show glucose-derived production of NAD in cortex and GBM. (A) Schematic of carbon incorporation into NAD and NADH. (B) ¹³C enrichment of NAD and NADH in cortex and GBM tissue from orthotopic GBM-bearing mice infused with [U¹³C]-glucose. Data are shown as average±standard deviation. N=7 mice. **p<0.01. Abbreviations: NAM, nicotinamide; ATP, adenosine triphosphate; NAD, oxidized nicotinamide adenine dinucleotide; NADH, reduced nicotinamide adenine dinucleotide.

FIGS. 24A-24C show time-course incorporation of [U¹³C]-glucose carbons into nucleotide metabolites in cortex and GBM. (A) Time-dependent enrichment profiles of purine metabolites IMP, GDP, guanosine, AMP, and inosine in cortex and GBM in orthotopic brain tumor bearing mice infused with [U¹³C]-glucose. (B) Time-dependent enrichment of pyrimidine metabolites UMP and uridine in cortex and GBM in orthotopic brain tumor bearing mice infused with [U¹³C]-glucose. (C) Relative abundance of nucleotide species in cortical and GBM tissues from orthotopic tumor bearing mice. Data are shown as average±standard deviation, n=3-9 samples from 1-3 mice per time point.

FIGS. 25A-25B show pathway based framework for in vivo modeling of nucleotide synthesis. Biochemical interconversions and model boundaries used for metabolic modeling of (A) purine synthesis and (B) pyrimidine synthesis.

FIGS. 26A-26F show decreased glucose-derived TCA cycle activity in GBM compared to cortex. Isotopologue abundance levels of citrate in cortex (A) and GBM (B) tissues at indicated time points from orthotopic GBM bearing mice infused with [U¹³C]-glucose. (C) Percent change in indicated citrate isotopologues from 120 to 240 min. Error bars are propagated from uncertainty in t=120 and t=240 min datapoints in (A) and (B). Isotopologue abundance levels of succinate in cortex (D) and GBM (E) in mice infused with [U¹³C]-glucose. (F) Percent change in indicated succinate isotopologues from 120 to 240 min. Error bars are propagated from uncertainty in t=120 and t=240 min datapoints in (D) and (E). Data are shown as average±standard deviation, n=6-9 samples from 2-3 mice per time point.

FIGS. 27A-27B show time-dependent changes in purine synthesis activity after RT in cortex and GBM. (A) Isotope enrichment and (B) relative abundance of purine metabolites IMP, GDP, guanosine, AMP, and inosine in cortex and GBM in orthotopic brain tumor bearing mice treated with a single dose of cranial RT (8 Gy) and then immediately infused with [U¹³C]-glucose. Data are shown as average±standard deviation, n=3-9 samples from 1-3 mice per time point.

FIG. 28 shows metabolic reaction fluxes after RT in GBM and cortex. Flux values were approximated using time-course enrichment profiles from mice infused with [U¹³C]-glucose as described in supplementary methods.

FIGS. 29A-29B show different serine sources in human cortex and brain cancers. (A) Statistical analysis of M+1 and M+3 serine enrichment confirms higher M+1 serine and lower M+3 serine in brain cancer compared to the cortex. Data are shown as average±standard deviation. *p<0.05. **p<0.01. ***p<0.001. ****p<0.0001. (B) Ratio of contribution of de novo serine synthesis to serine salvage in human cortex and brain cancers. A value higher than 1 indicates that de novo synthesis is the predominant source of labeled serine, while a value lower than 1 indicates that serine salvage is the predominant source of labeled serine. The error bars represent 95% confidence intervals.

FIGS. 30A-30C show plasma serine levels and tissue metabolites in orthotopic GBM bearing mice on a serine/glycine-restricted diet. (A) Levels of serine in plasma from orthotopic GBM bearing mice fed either a control diet or serine/glycine-restricted diet, n=5 mice per group. (B) Principal component analysis of cortex and GBM tissues from mice on a control diet (n=4) or serine/glycine-restricted diet (n=5) in panel A. (C) Individual metabolite levels in cortical tissue from mice in panel A. One mouse in the control diet group in panel A was found to have no detectable tumor and was therefore excluded from analyses in panels B and C.

FIG. 31 shows metabolic fluxes generated through a constrained optimization problem and used to simulate time-course MIDs. The simulated MIDs were split into training, validation, and test datasets. Validation dataset (Val.) was used to tune hyperparameters in a Bayesian optimization approach (BO) with an objective function set to coefficient of determination (CD) between predicted and actual values. Test dataset was used to evaluate model on unseen data (Eval.). (2) Three machine learning models were implemented in this study including two convolutional neural networks (CNNs) and a graph neural network (GNN). (3) The machine learning models were validated experimentally. (4) Machine learning models were administered to predict the ratio of de novo GMP synthesis in patients. (5) The predicted ratio was further validated by flux balance analysis of patient scRNA-seq data. (6) The machine learning model will be used in a clinical trial to suggest which patient may benefit from MMF treatment.

FIGS. 32A-32G show metabolic-based machine learning models. (A) Metabolic model used in the MID simulations and machine learning models. (B) Distribution of simulated fluxes based on stoichiometric mass balances. (C) Simulated data structure. M, i, and t represent metabolites, MIDs, and time, respectively. m, n, and k denote no. of metabolites, no. of MIDs, and no. of timepoints, respectively. (D) First CNN framework. (E) Second CNN framework. (F) Graph structure of purine metabolism. (G) GNN framework.

FIGS. 33A-33C show machine learning predictions. (A) First CNN predictions on training, validation, and test datasets. (B) Experimental validation of model performance on MMF treated PDXs vs control PDXs. (C) Model prediction on patient enhancing and non-enhancing tumors.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “absolute metabolic flux” as used herein refers to the total flux of molecules through a given metabolic pathway.

The term “isotopologues” as used herein refers to molecules that have the same chemical formula and bonding arrangement of atoms, but differ only in their isotopic composition. Isotopologues are also referred to as “mass isotopomers”. Isotopologues are generated by the methods described herein due to metabolization of the tracer (e.g. ¹³C, or other suitable tracers. Metabolization of the tracer leads to the generation of metabolites labeled with the tracer (e.g. with the ¹³C). For example, a metabolite with n carbon atoms can have 0 to n of its carbon atoms labeled with the tracer (e.g. with the ¹³C), resulting in isotopologues that increase in mass (M) from M+0 (all carbons unlabeled) to M+n (all carbons labeled).

The term “subject” is used herein in the broadest sense and refers to any organism in which metabolic flux can be assessed. In some embodiments, the subject is an animal. In some embodiments, the subject is a vertebrate. In some embodiments, the subject is a bird. In some embodiments, the subject is a mammal. Suitable mammals include, but are not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some embodiments, the subject is a plant.

### DETAILED DESCRIPTION

In some aspects, provided herein are methods for estimating absolute metabolic flux for a pathway of interest. In some embodiments, methods for estimating absolute metabolic flux for a pathway of interest comprise administering a tracer to a subject, collecting a sample from the subject, and generating an estimate of absolute metabolic flux for the pathway of interest. In some embodiments, the sample is a tissue sample.

In some embodiments, the estimate of absolute metabolic flux for the pathway of interest is generated based upon a level of one or more metabolites and/or isotopologues thereof in the sample at a single point in time following administration of the tracer to the subject. In some embodiments, the level of one or more metabolites and/or isotopologues is measured in the sample. For example, the level of the one or more metabolites and/or isotopologues thereof can be measured in the sample using any suitable technique, including mass spectrometry-based methods (e.g., liquid chromatography-mass spectrometry (LC-MS)), nuclear magnetic resonance (NMR) spectroscopy, or other suitable methods. In some embodiments, the level of a given metabolite or isotopologue thereof is measured in the sample at a point in time following administration of a tracer. In some embodiments, the level of a given metabolite or isotopologue thereof in the sample is measured at a point in time following administration of radiation therapy.

In some embodiments, the pathway of interest is a purine synthesis pathway. In some embodiments, the pathway of interest is a purine synthesis pathway and the one or more metabolites and/or isotopologues thereof comprise one or more of Ribose-5-Phosphate (R5P), Glycine (GLY), Carbon Dioxide (CO2), 5-Methyltetrahydrofolate (C-THF), Inosine Monophosphate (IMP), Inosine, Hypoxanthine, Guanine, Guanosine Monophosphate (GMP), Guanosine Diphosphate (GDP), Guanosine, Adenosine Monophosphate (AMP), and Adenosine. In some embodiments, the one or more metabolites and/or isotopologues thereof comprise at least 2 of, at least 3 of, at least 4 of, at least 5 of, at least 6 of, at least 7 of, at least 8 of, at least 9 of, at least 10 of, at least 11 of, at least 12 of, or each of Ribose-5-Phosphate (R5P), Glycine (GLY), Carbon Dioxide (CO2), 5-Methyltetrahydrofolate (C-THF), Inosine Monophosphate (IMP), Inosine, Hypoxanthine, Guanine, Guanosine Monophosphate (GMP), Guanosine Diphosphate (GDP), Guanosine, Adenosine Monophosphate (AMP), and Adenosine.

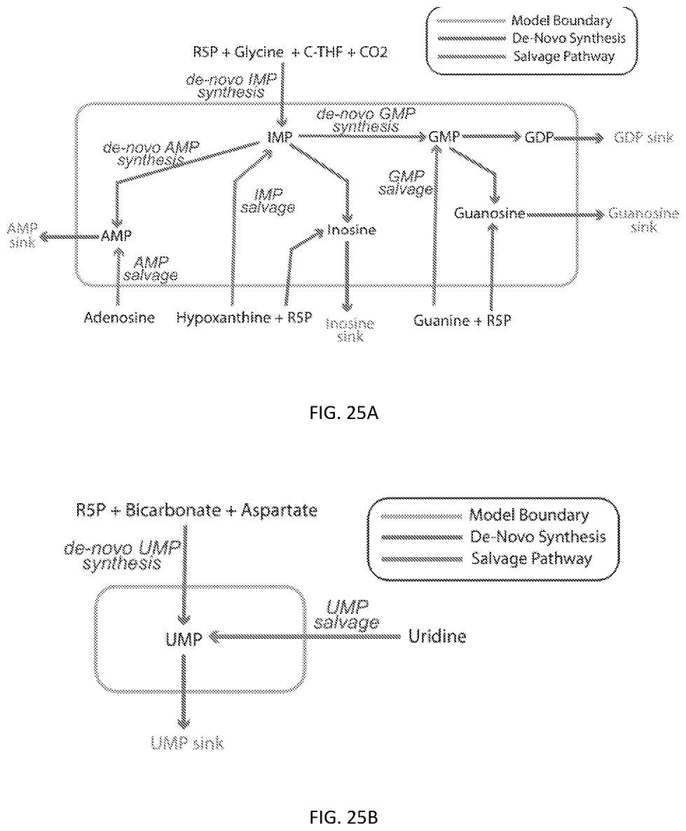

In some embodiments, the purine synthesis pathway comprises multiple reactions. Exemplary reactions in the purine synthesis pathway are shown in FIG. 25A. In some embodiments, generating an estimate of absolute metabolic flux for the purine synthesis pathway comprises determining the total flux through multiple reactions involved in the purine synthesis pathway. For example, in some embodiments generating an estimate of absolute metabolic flux for the purine synthesis pathway comprises determining the total flux through each of the reactions shown in FIG. 25A. In some embodiments, the method further comprises determining a contributing fraction of de novo GMP synthesis and/or de novo IMP synthesis in the purine synthesis pathway. The “contributing fraction” refers to the portion of the estimate of absolute metabolic flux which can be attributed to (e.g. which is contributed by) de novo GMP synthesis and/or de novo IMP synthesis.

In some embodiments, the pathway of interest is the pyrimidine synthesis pathway. In some embodiments, the pathway of interest is the pyrimidine synthesis pathway and the one or more metabolites and/or isotopologues thereof comprise one or more of Ribose-5-Phosphate (R5P), Aspartate (ASP), Carbon Dioxide (CO2), Uridine, and Uridine Monophosphate (UMP). In some embodiments, the one or more metabolites and/or isotopologues thereof comprise at least 2 of, at least 3 of, at least 4 of, or each of Ribose-5-Phosphate (R5P), Aspartate (ASP), Carbon Dioxide (CO2), Uridine, and Uridine Monophosphate (UMP). In some embodiments, the pyrimidine synthesis pathway comprises multiple reactions. Exemplary reactions in the pyrimidine synthesis pathway are shown in FIG. 25B. In some embodiments, generating an estimate of absolute metabolic flux for the pyrimidine synthesis pathway comprises determining the total flux through multiple reactions involved in the pyrimidine synthesis pathway. For example, in some embodiments generating an estimate of absolute metabolic flux for the pyrimidine synthesis pathway comprises determining the total flux through each of the reactions shown in FIG. 25B.

In some embodiments, the pathway of interest is a serine synthesis pathway (e.g. a de novo serine synthesis pathway). In some embodiments, the pathway of interest is a serine synthesis pathway and the one or more metabolites and/or isotopologues thereof comprise one or more of 3-phosphoglycerate (3PG), glycine (gly), and 5,10-methylene-THF (Me-THF).

In some embodiments, the level or the one or more metabolites and/or isotopologues thereof is increased at the time of measurement. The estimate of the absolute metabolic flux for the pathway of interest is therefore also described herein as being generated base upon a measured enrichment in one or more isotopologues of at least one metabolite of interest at a given point in time (e.g. following administration of the tracer, and/or following administration of radiation therapy to the subject). Accordingly, measuring the level of the one or more metabolites and/or isotopologues thereof is also referred to herein as measuring “enrichment” in one or more isotopologues of at least one metabolite of interest at a given point in time following administration of radiation therapy to the subject. For example, measuring “enrichment” or the level being “enriched” can refer to an increase in a level of a given metabolite or isotopologue thereof at a given point in time (e.g. at the time of measurement) compared to another point in time or compared to a baseline level. In some embodiments, the baseline level is measured. For example, in some embodiments the baseline level is measured prior to radiation and/or prior to administration of the tracer to the subject. In some embodiments, the baseline level is obtained through other means, such as an estimation of the amount of a given metabolite in a specific tissue based upon knowledge in the literature or based upon measurements extrapolated from other sources.

In some embodiments, the estimate of absolute metabolic flux for the metabolite of interest is generated using an artificial intelligence/machine learning (AI/ML) model. An AI/ML model refers to a mathematical algorithm trained using data and/or human input to make predictions. The AI/ML model may use any one or more algorithms, including linear regression, logistic regression, linear discriminant analysis, decision trees, Naive Bayes, K-Nearest Neighbors, learning vector quantization, support vector machines, bagging and random forest, and/or neural networks (e.g. Deep neural networks). Exemplary algorithms and equations that can be used in estimating the absolute metabolic flux are shown in the accompanying examples.

In some aspects, provided herein is a machine-learning method for estimating absolute activity of one or more metabolic fluxes. In some embodiments, the machine-learning method is used for estimating the absolute activity of a single enzyme (e.g. estimating the absolute activity of a single enzyme in a given metabolic pathway). In some embodiments, the machine-learning method is used for estimating the absolute activity of a metabolic pathway (e.g. a pathway influenced by a plurality of enzymes).

In some embodiments, the machine-learning method for estimating absolute activity of one or more metabolic fluxes comprises obtaining a plurality of measurements from a sample collected from a subject, and applying an artificial intelligence/machine learning model to the measurements to generate an estimate of absolute activity of one or more metabolic fluxes.

In some embodiments, each measurement is a level of a metabolite and/or an isotopologue thereof in a metabolic pathway of interest at a single point in time. The level of the one or more metabolites and/or isotopologues thereof can be measured in the sample using any suitable technique, including mass spectrometry-based methods (e.g., liquid chromatography-mass spectrometry (LC-MS), nuclear magnetic resonance (NMR) spectroscopy, or other suitable methods. In some embodiments, the plurality of measurements are obtained at a single point in time after administration of a tracer to the subject. In some embodiments, the plurality of measurements are obtained at a single point in time after administration of radiation therapy to the subject.

In some embodiments, the level or the one or more metabolites and/or isotopologues thereof is increased at the time of measurement. Accordingly, the method may also be described herein as involving obtaining a plurality of measurements from the sample, wherein each measurement is a measurement of “enrichment” in one or more metabolites or isotopologues thereof. For example, measuring “enrichment” or the level being “enriched” can refer to an increase in a level of a given metabolite or isotopologue thereof at a given point in time (e.g. at the time of measurement) compared to another point in time or compared to a baseline level. In some embodiments, the baseline level is measured. For example, in some embodiments the baseline level is measured prior to radiation and/or prior to administration of the tracer to the subject. In some embodiments, the baseline level is obtained through other means, such as an estimation of the amount of a given metabolite in a specific tissue based upon knowledge in the literature or based upon measurements extrapolated from other sources.

In some embodiments, the method further comprises applying an artificial intelligence/machine learning (AI/ML) model to the measurements to generate an estimate of absolute activity of one or more metabolic fluxes. The AI/ML model may use any one or more algorithms, including linear regression, logistic regression, linear discriminant analysis, decision trees, Naive Bayes, K-Nearest Neighbors, learning vector quantization, support vector machines, bagging and random forest, and/or neural networks (e.g. Deep neural networks). In some embodiments, the AI/ML model uses a neural network.

For any of the embodiments herein, the subject may be affected with or at risk of having a disease or condition for which assessment of metabolic flux may be useful, such as for guiding therapy selection for the disease or condition. In some embodiments, the subject is affected with or suspected of having cancer. In some embodiments, the subject has cancer, and the tissue sample comprises a tumor tissue sample. In some embodiments, the cancer is a brain cancer. In some embodiments, the brain cancer is a glioblastoma. In some embodiments, the subject is a candidate for radiation therapy treatment or has received radiation therapy. In some embodiments, the estimate of absolute metabolic flux for the pathway of interest is generated based upon a level of one or more metabolites and/or isotopologues thereof at a single point in time following administration of radiation therapy to the subject.

In some embodiments, the estimate of absolute metabolic flux for the metabolic pathway of interest can be used for personalized medicine, including guiding therapy selection for a subject having cancer (e.g. having brain cancer, including glioblastoma). For example, subjects may be identified as likely to be sensitive to or resistant to a given treatment targeting a given metabolic pathway based upon the estimate of absolute metabolic flux generated for the subject using the methods described herein. Accordingly, in some aspects provided herein are methods of selecting a therapy for a subject in need thereof, including a subject having cancer. The methods comprise generating an estimate of absolute metabolic flux, using methods described herein, and selecting the appropriate therapy (e.g. anti-cancer therapy) for the subject based upon the estimate of absolute metabolic flux. The term “selecting” is used in the broadest sense and includes identifying the patient as sensitive to a therapy, recommending to the subject a therapy, and/or administering to the subject a therapy. In some embodiments, the therapy is an IMPDH inhibitor. In some embodiments, an increased contribution of de novo GMP synthesis and/or increased de novo IMP synthesis in a subject (e.g. in a sample obtained from a subject and assessed by the methods described herein) indicates that the subject is sensitive to treatment with an IMPDH inhibitor. In some embodiments, the therapy is dietary restriction of amino acids. In some embodiments, the therapy is dietary restriction of amino acids such as serine and glycine. In some embodiments, the therapy is dietary serine restriction. Dietary serine restriction is inclusive of dietary restriction of serine and any additional amino acids (e.g. glycine). In some embodiments, the therapy is dietary serine and glycine restriction. In some embodiments, decreased serine synthesis in a subject indicates that a subject is sensitive to dietary restriction of amino acids (e.g. dietary serine restriction or dietary serine and glycine restriction).

In some embodiments, the estimate of the absolute activity of one or more metabolic fluxes is used for personalized medicine, including guiding therapy selection for a subject having cancer. For example, subjects may be identified as likely to be sensitive to or resistant to a given treatment targeting a given metabolic pathway based upon the estimate of the absolute activity of a metabolic flux (e.g. the activity of a given enzyme in a metabolic pathway) determined using the methods described herein. Accordingly, in some aspects provided herein are methods for selecting an appropriate therapy for a subject in need thereof, including an appropriate anti-cancer therapy for a subject. The methods comprise estimating the absolute activity of one or more metabolic fluxes in a subject using the methods described herein, and selecting the appropriate therapy (e.g. anti-cancer therapy) based upon the estimated absolute activity.

In some aspects, the methods provided herein find use in determining which subjects will be sensitive to treatment with an IMPDH inhibitor or an agent which targets de novo GMP synthesis. In some embodiments, subjects (e.g. subjects having brain cancer, including subjects having glioblastoma) having increased de novo GMP synthesis (or increased de novo IMP synthesis, which is a precursor for de novo GMP synthesis) are identified as sensitive to treatment with an IMPDH inhibitor or an agent that targets the de novo pathway, such as 5-fluorocil. In some embodiments, provided herein is a method of selecting a therapy for a subject having cancer, the method comprising determining a contributing fraction of de novo GMP synthesis and/or de novo IMP synthesis in a purine synthesis pathway, and selecting a inosine monophosphate dehydrogenase (IMPDH) inhibitor for the subject when the contributing fraction of de novo GMP synthesis and/or de novo IMP synthesis is increased relative to a threshold value. The threshold value may be a value of de novo GMP and/or de novo IMP synthesis in control subjects or from control tissue (e.g. tissue not affected by the cancer). In some embodiments the IMPDH inhibitor a reversible inhibitor, a synthetic non-nucleoside inhibitor, a non-nucleoside natural product inhibitor, a parasite-selective IMPDH inhibitor, a reversible nucleoside inhibitor, or a mechanism-based inactivator of IMPDH. In some embodiments, the IMPDH inhibitor is mycophenolate mofetil (MMF), mycophenolic acid (MPA) or a derivative thereof, tiazofurin, ribavirin, VX-994, VX-497, FF-10501, thiazole-4-carboxamide adenine dinucleotide (TAD), nicotinamide adenine dinucleotide (MAD), benzmide riboside, mizorbine, EICAR, selenazofurin, thiophenfurin, myricetin, gnidilatimonoein, VS-148, BMS-566419, BMS-337197, or AS2643361. In some embodiments, the method further comprises administering the IMPDH inhibitor to the subject.

In some aspects, the methods provided herein find use in determining which subjects will be sensitive to treatment with dietary restriction of amino acids (e.g. dietary serine restriction or dietary serine and glycine restriction). In some embodiments, subjects (e.g. subjects having brain cancer, including subjects having glioblastoma) having decreased de novo serine synthesis are identified as sensitive to treatment dietary serine restriction. In some embodiments, provided herein is a method of selecting a therapy for a subject having cancer, the method comprising determining absolute activity of a serine synthesis pathway (e.g. de novo serine synthesis) and selecting dietary serine restriction for the subject when the absolute activity of a serine synthesis pathway is decreased relative to a threshold value. The threshold value may be a value of serine synthesis in control subjects or from control tissue (e.g. tissue not affected by the cancer).

For any of the methods described herein, the term “tracer” is used in the broadest sense and refers to any moiety comprising a label that can be incorporated into a metabolite over the course of a metabolic process. In some embodiments, tracer comprises a moiety labeled with ¹³C, ¹⁵N, ¹⁸O, or ²D. The moiety may be any suitable moiety, including but not limited to glucose, methionine, serine, glutamine, lactate, acetate, hypoxanthine, uridine, and water. In some embodiments, the moiety is glucose. In some embodiments, the tracer comprises ¹³C-glucose.

For any of the methods described herein, the subject can be any subject, including human and non-human subjects. In some embodiments, the subject is a vertebrate. In some embodiments, the subject is a mammal. In some embodiments, the subject is a human.

In some embodiments, a sample is obtained from a subject and the level of one or more metabolites and/or isotopologues from the sample are analyzed. In some embodiments, the sample comprises tumor tissue (e.g., a biopsy, removed tumor tissue from surgery, etc.). In some embodiments, tracer is administered to a subject prior to or during a procedure that removes sample from a subject. For example, tracer may be administered one or more minutes or hours prior to a procedure. Tracer may be administered via any suitable manner. Suitable routes of administration include, but are not limited to, oral, sublingual, buccal, rectal, vaginal, ocular, nasal, transdermal, parenteral, inhalation, and the like. In some embodiments, the tracer is administered orally. In some embodiments, the tracer is administered parenterally (e.g. by injection, including intravenous, intramuscular, intrathecal, subcutaneous, intraparenchymal, intracerebroventricular, etc.).

For any of the methods described herein, the level of the one or more metabolites and/or isotopologues thereof can be measured at any suitable point in time following administration of the tracer. For example, in some embodiments the in the sample about 1 minute to about 5 hours after administration of the tracer. For example, in some embodiments the level is measured about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 120 minutes, about 2.5 hours, about 3 hours, about 3.5 hours, about 4 hours, about 4.5 hours, or about 5 hours after administration of the tracer.

For any of the methods described herein, the level of the one or more metabolites and/or isotopologues thereof can be measured at any suitable point in time following administration of radiation therapy to the subject. For example, in some embodiments the in the sample about 1 minute to about 5 hours after administration of radiation therapy to the subject. For example, in some embodiments the level is measured about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 120 minutes, about 2.5 hours, about 3 hours, about 3.5 hours, about 4 hours, about 4.5 hours, or about 5 hours after administration of radiation therapy to the subject.

### EXAMPLES

### Example 1

### In Vivo Model Systems

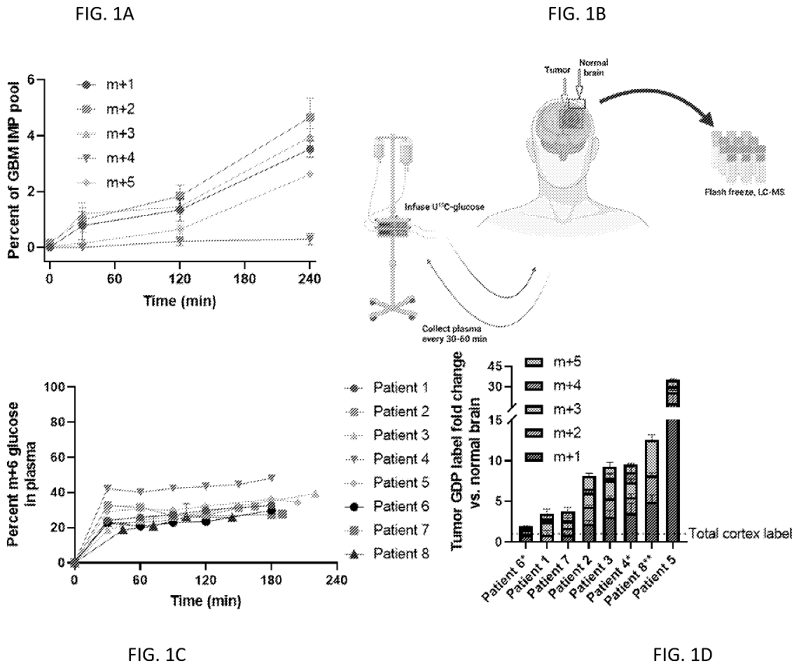

To generate data used for flux modeling, patient-derived xenografts (PDXs) from human glioblastomas are implanted into the brains of immunodeficient mice. PDXs are transduced with luciferase and EGFP prior for imaging and physical separation from brain tissue, respectively. Once brain tumors are established, mice are cannulated for infusions, treatments, and blood sampling. One catheter is surgically placed into the jugular vein of anesthetized recipient mice, and a second line is placed into the carotid artery. In experiments in which mice are to receive oral administration of MMF or vehicle, a third catheter is directed to the stomach. After recovery from surgery, mice are treated with cranial radiation therapy and/or MMF (via gastric line) and then infused with uniformly labeled ¹³C-glucose through the jugular vein. Infusions are performed while mice are awake and active. Aliquots of blood are collected from the carotid line every 5-60 minutes in EDTA-coated tubes for plasma preparation. At the end of infusions, mice are sacrificed, and tissues are collected. Brain tumors are separated from healthy brain tissue by fluorescence-guided microdissection. All samples are immediately flash frozen in liquid nitrogen and stored at −80 degrees Celsius. Small molecules from solid tissues and plasma are extracted and samples are analyzed by liquid chromatography-mass spectrometry (LC-MS) to identify the metabolites and their labeling patterns (FIG. 1A).

### Human Isotope Labeling

Patients with suspected GBM are infused with uniformly labeled ¹³C-glucose via IV line during clinically indicated resection (FIG. 1B). Blood is collected from the arterial line before and at regular time points during infusion (FIG. 1C). When tumor and adjacent normal brain tissue are resected by the neurosurgeon, resected tissues are immediately flash frozen in liquid nitrogen in the operating room (<2 min after tissue removal). Metabolites are then extracted and metabolite labeling is assessed by LC-MS as (FIG. 1D) as with mouse models described above.

### Quantification of Absolute Metabolic Fluxes in the Purine Pathway

### Model Setup and Optimization

To quantify the fluxes in the purine pathway, an Isotopic Nonstationary Metabolic Flux Analysis (INST-MFA) methodology was developed. This method addresses several challenges in in-vivo flux quantification: (1) The metabolite uptake and secretion fluxes (i.e. exchange fluxes) cannot be measured experimentally. (2) In-vivo experiments are resource extensive and can only be performed for a few hours, which is not enough to achieve isotopic steady state. (3) Inter-organ metabolism leads to secondary enrichment in circulating metabolites. To overcome these challenges, a piecewise linear function was fit to the experimentally obtained enrichments of the metabolites that are taken up from outside the reaction network (designated “external metabolites”, equation 1).

Isotopic mass balance is applied to the metabolites within the reaction network (designated “internal metabolites”) to generate a system of ordinary differential equations (ODEs) (equation 2). These ODEs are solved along with linear mass balance constraints and physiologically defined parameter bounds (equation 3). The parameter vector comprises of the network fluxes, the pool sizes of the internal metabolites, and the time-course enrichment of the external metabolites. To select the optimum parameters, time-course enrichment of all metabolites (equations 1 and 2) is simulated and an objective function is defined to evaluate model fit to the experimental data. The objective function is defined as the sum of squared errors between the simulated and experimental values scaled by the standard deviation of the experimental value (equation 4).

These experimental values include enrichments of internal and external metabolites, as well as the known pool sizes. The pool sizes of the metabolites in mouse brain is obtained from literature (table 1) and the corresponding pool sizes on the GBM tissue is estimated based on the relative peak areas from mass spectrometry experiments. The Artelys Knitro solver is used to implement an iterative local optimization approach based on the interior-point/conjugate-gradient algorithm (Byrd, R. H. et al. (2006) KNITRO: An integrated package for nonlinear optimization. Nonconvex Optim 83, 35; Byrd, R. H. et al. (1999) An interior point algorithm for large-scale nonlinear programming. Siam J Optimiz 9, 877-900.) Local optimizations are performed from 100 randomly assigned initial points and the set of parameters with the lowest objective function within the statistically acceptable range as defined by a chi-squared distribution is selected. A parameter sensitivity approach is implemented to estimate the 95% confidence intervals for the optimized parameters as described previously (Antoniewicz, M. R. et al. (2006) Determination of confidence intervals of metabolic fluxes estimated from stable isotope measurements. Metabolic Engineering 8, 324-337).

TABLE 1
Concentration of purine metabolites in mouse brain

| Metabolite | Concentration ± SD (pmol/mg tissue) | Source |
|---|---|---|
| Inosine | 126.7 ± 18.3 | [4] |
| GMP | 157 ± 40 | [5] |
| GDP | 182.1 ± 8.2 | [6] |
| Guanosine | 245.8 ± 31.6 | [4] |
| AMP | 172.1 ± 6 | [6] |

### Model Equations for INST-MFA

$$\frac{{dx}_{i , M + p} ( t )}{dt} = \{ \begin{matrix}\frac{{x}_{i , M + p} ( {t}_{2} ) - {x}_{i , M + p} ( {t}_{1} )}{{t}_{2} - {t}_{1} \\ …} & {t}_{1} \leq t < {t}_{2} \\ \frac{{x}_{i , M + p} ( {t}_{m + 1} ) - {x}_{i , M + p} ( {t}_{m} )}{{t}_{m + 1} - {t}_{m}} & {t}_{m} \leq t < {t}_{m + 1}\end{matrix} \qquad Equation 1$$

$$\frac{{dy}_{i , M + p}}{dt} = ( {\sum }_{{{S}_{ij} > 0}^{j = 1}}^{N} {S}_{i j} \cdot {v}_{j} ( {\prod }_{{{S}_{kj} < 0}^{{Σ q = p}^{k}}} {y}_{k , M + q} ) + {\sum }_{{{S}_{ij} < 0}^{j = 1}}^{N} {S}_{i j} \cdot {v}_{j} \cdot {y}_{i , M + p} ) \cdot \frac{1}{{c}_{i}} \qquad Equation 2$$

$$S . v = 0 \qquad Equation 3$$

$$obj = \sum {( \frac{{y}_{s i m} - {y}_{expt}}{S {D}_{expt}} )}^{2} + \sum {( \frac{{c}_{s i m} - {c}_{expt}}{S {D}_{expt}} )}^{2} \qquad Equation 4$$

A description of the variables in equations 1˜4 is provided in table 2.

TABLE 2
Description of variables in INST-MFA equations 1-4.

| Variable | Description |
|---|---|
| x_{i, M + p}(t) | Enrichment of the M + p^{th} isotopologue of the i^{th} external |
| | metabolite at time t |
| y_{i, M + p} | Enrichment of the M + p^{th} isotopologue of the i^{th} internal |
| | metabolite at time t |
| v | Flux vector |
| S | Stoichiometric matrix |
| cᵢ | Concentration of i^{th} internal metabolite |
| vⱼ | Flux of the j^{th} reaction |
| Sᵢⱼ | Stoichiometric coefficient of the i^{th} metabolite and j^{th} reaction |
| y_{k, M + q} | Enrichment of the M + q^{th} isotopologue of the k^{th} internal or |
| | external metabolite s.t. q ≤ p |
| N | Number of reactions in the model |
| yₛᵢₘ | Simulated enrichment |
| yₑₓₚₜ | Experimentally determined enrichment |
| cₛᵢₘ | Simulated concentration of internal metabolite |
| cₑₓₚₜ | Experimentally determined concentration of internal |
| | metabolite |
| SDₑₓₚₜ | Standard deviation of experimental value |

### Reaction Network and Results

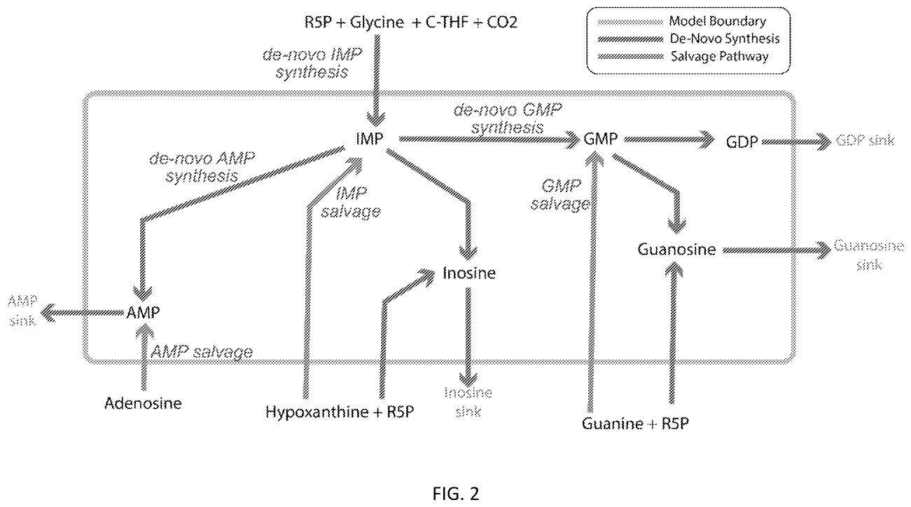

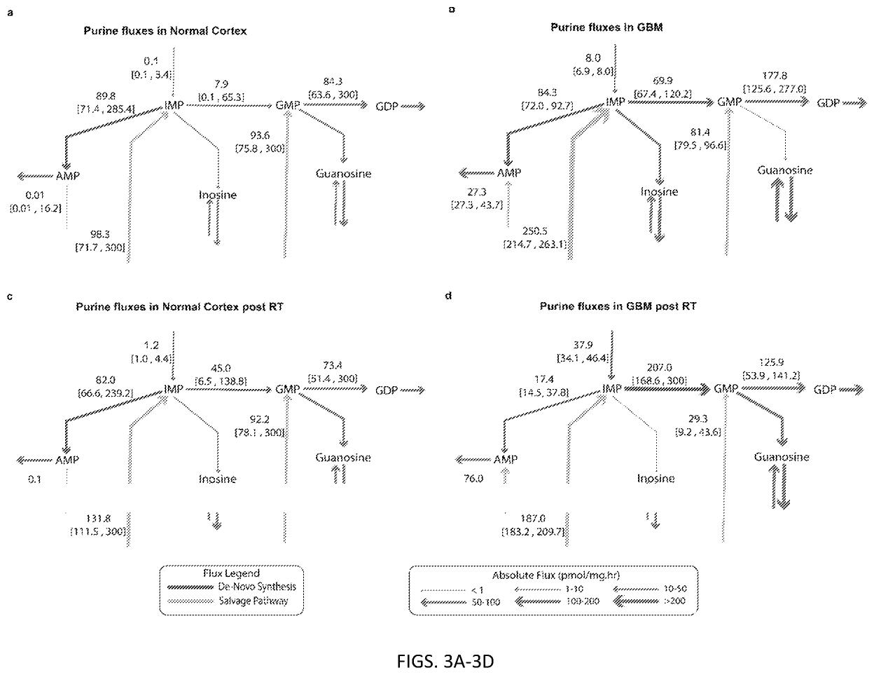

The metabolic network comprises of 15 reactions and 13 metabolites in the purine pathway (FIG. 2). 6 metabolites are designated as internal metabolites: inosine monophosphate (IMP), inosine, guanosine, guanosine monophosphate (GMP), guanosine diphosphate (GDP), and adenosine monophosphate (AMP). 7 metabolites are designated as external metabolites: ribose-5-phosphate (R5P), glycine, formyl-THF, carbon dioxide, hypoxanthine, guanine, and adenosine. Out of these, only R5P, glycine, and formyl-THF have experimentally measured isotopic enrichment. The quantified fluxes are depicted in FIG. 3.

Quantification of Dynamic Metabolic Fluxes after Radiation

### Model Setup and Optimization

A dynamic MFA model was built to quantify the flux profiles in the purine pathway after radiation (equations 1, 5, 6). The time-dependent enrichment of external metabolites is described with piecewise linear functions as in the case of INST-MFA (equation 1). In addition to using time-dependent enrichment and initial metabolite pool sizes in the model, the time-dependent change in the concentration of the internal metabolites was included in the model objective (equation 7). The in-vivo INST-MFA method was modified by describing the fluxes as time-dependent b-splines (Martinez, V. S. et al. (2015) Metab Eng Commun 2, 46-57; Quek, L. E. et al. (2020) iScience 23, 100855). Through hit-and-trial, third order b-splines were determined to be appropriate to model the fluxes in this system. An iterative approach to optimize the number and position of b-spline internal knots is used. Once the knot position(s) are selected, local optimization from 100 initial guesses is performed. The flux solutions from the INST-MFA model was also included for both the control and RT conditions in the set of initial guesses. The set of parameters with the lowest objective value is selected and a likelihood-based approach is applied to estimate the 95% confidence intervals (Kreutz, C. et al. (2012) Likelihood based observability analysis and confidence intervals for predictions of dynamic models. BMC Syst Biol 6, 120. 10.1186/1752-0509-6-120).

### Model Equations for Dynamic-MFA

$$\frac{d c ( t )}{dt} = S * v ( t ) \qquad Equation 5$$

$${y}_{i , M + p} \frac{{dc}_{i}}{dt} + {c}_{i} \frac{{dy}_{i , M + p}}{dt} = {\sum }_{{{S}_{ij} > 0}^{j = 1}}^{N} {S}_{i j} \cdot {v}_{j} ( {\prod }_{{{S}_{kj} < 0}^{{\sum q = p}^{k}}} {y}_{k , M + q} ) + {\sum }_{{{S}_{ij} < 0}^{j = 1}}^{N} {S}_{i j} \cdot {v}_{j} \cdot {y}_{i , M + p} \qquad Equation 6$$

$$obj = \sum {( \frac{{y}_{s i m} - {y}_{expt}}{S {D}_{expt}} )}^{2} + \sum {( \frac{{c}_{0 , s i m} - {c}_{0 , expt}}{S {D}_{expt}} )}^{2} + \sum {( \frac{{( \frac{{c}_{t}}{{c}_{0}} )}_{s i m} - {( \frac{{c}_{t}}{{c}_{0}} )}_{expt}}{S {D}_{expt}} )}^{2} \qquad Equation 7$$

A description of the variables in equations 5-7 is provided in table 3.

TABLE 3
Description of variables in Dynamic-MFA equations 5-7.

| x | Description |
|---|---|
| c(t) | Concentration vector for internal metabolites at time t |
| v(t) | Flux vector at time t |
| S | Stoichiometric matrix |
| cᵢ | Concentration of metabolite i |
| vⱼ | Flux of the j^{th} reaction |
| Sᵢⱼ | Stoichiometric coefficient of the i^{th} metabolite and j^{th} reaction |
| y_{i,M+p} | Enrichment of the M + p^{th} isotopologue of the i^{th} internal metabolite at time t |
| y_{k,M+q} | Enrichment of the M + q^{th} isotopologue of the k^{th} internal or external |
| | metabolite s.t. q ≤ p |
| N | Number of reactions in the model |
| yₛᵢₘ | Simulated enrichment |
| yₑₓₚₜ | Experimentally determined enrichment |
| c_{0,sim} | Simulated concentration of internal metabolites at t = 0 |
| c_{0,expt} | Experimentally obtained concentration of internal metabolites at t = 0 |
| ${( \frac{{c}_{t}}{{c}_{0}} )}_{s i m}$ | Simulated ratio between concentration of internal metabolite at time t and the concentration at t = 0 |
| ${( \frac{{c}_{t}}{{c}_{0}} )}_{expt}$ | Experimentally determined ratio between concentration of internal metabolite at time t and the concentration at t = 0 |
| SDₑₓₚₜ | Standard deviation of experimental value |

### Reaction Network and Results

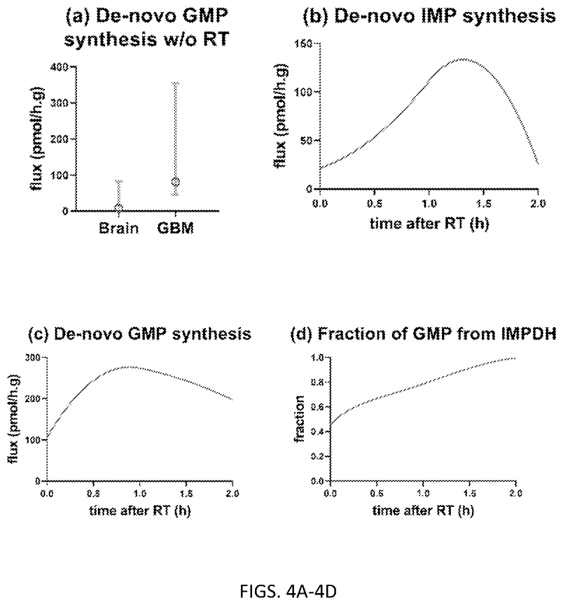

The reaction network is the same as the one used for INST-MFA (FIG. 2). The best-fit time-course profiles of the purine pathway fluxes are depicted in FIG. 4A-FIG. 4D, further described in Example 2.

### Machine Learning to Estimate Relative GMP Synthesis in Human Subjects

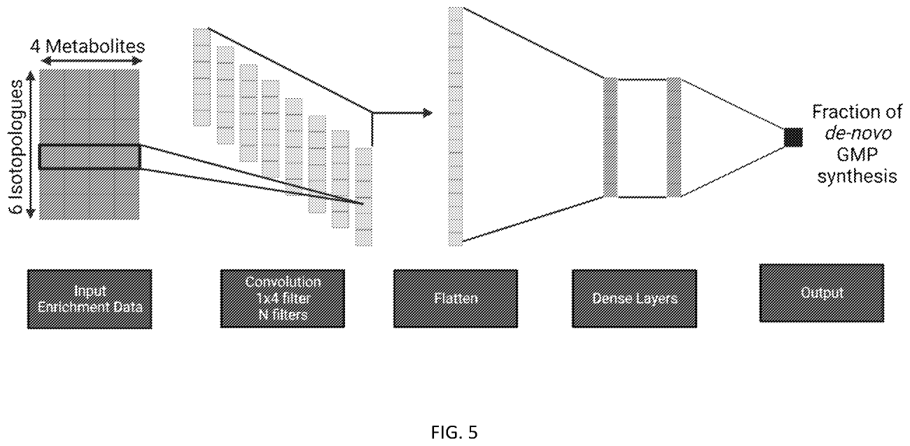

A machine-learning based method to estimate the fraction of GMP that is synthesized de novo in patient GBMs is described herein. This information is used to identify patients whose tumors have higher contribution of IMPDH in GMP synthesis are expected to benefit more from MMF treatment. A significant challenge in estimating the fluxes in human subsets is that we are limited to enrichment data from a single measurement at isotopic non-steady state. To overcome this challenge, the ODE-based model is used to simulate metabolite enrichments for different flux vectors, and then this data is used to fit a convolutional neural network (CNN) model (FIG. 5). The model inputs can be the experimentally obtained enrichments of purine metabolites, and the model output can be the fraction of de novo GMP synthesis. The CNN architecture is set up to capture the relationship between the first 6 isotopologues of four metabolites in the purine pathway: IMP, R5P, GDP, and guanosine. These metabolites are selected on the quality of the metabolomics data. A 1×4 filter is used for data convolution. The number of filters are optimized as a hyperparameter. An exemplary architecture for the CNN model is shown in FIG. 5.

### Example 2

Glioblastoma (GBM) is the most common aggressive brain cancer in adults and has a five-year survival rate of less than 10%. Radiation is a common treatment modality for GBM patients but resistance to radiation is common. Radiation-resistant GBMs have high levels of the purine nucleoside guanosine triphosphate (GTP). GTP may promote survival of glioblastoma tissue after radiation (e.g. may inhibit the ability of radiation to kill glioblastoma cells) by enhancing DNA repair. Inhibition of GTP synthesis by the FDA-approved drug mycophenolate (MMF), an inosine monophosphate dehydrogenase (IMPDH) inhibitor, improves radiation outcomes in mice models (Zhou W. et al., Nat Commun. (2020); 11(1):3811)).

The IMPDH enzyme facilitates de novo GMP (guanosine monophosphate) synthesis and is a promising target for tumors with high flux through IMPDH. However, methods for quantifying tumor IMPDH flux are lacking. Improved methods for quantifying tumor IMPDH flux would identify patients that would benefit from cancer treatment with MMF or other IMPDH inhibitors. For example, patients identified as having high tumor IMPDH flux would likely be sensitive to treatment with an IMPDH inhibitor following radiation in order to enhance efficacy of radiation treatment.

Described herein is the development and use of a method for in-vivo metabolic flux analysis. Specifically, ¹³C-metabolic flux analysis (MFA) was used in GBM xenograft models to quantify fluxes in the purine pathway before and after radiation treatment. Further, ¹³C-glucose tracing was performed in glioblastoma patients to estimate IMPDH activity in patient tumors.

Materials and Methods: GBM mice models were infused with ¹³C glucose as a tracer for different time points. GBM and normal brain tissue were analyzed via mass spectrometry to obtain the ¹³C-enrichment time course profiles of intracellular metabolites. A system of ordinary differential equations was fit based on least squares optimization to estimate purine pathway fluxes with and without radiation treatment. Post-radiation change in metabolite levels was used to estimate the dynamic fluxes with time.

For glioblastoma patients, ¹³C-glucose was infused (intravenously) during tumor resection surgery. Resected tumor tissue was analyzed via mass spectrometry to obtain the ¹³C-enrichment time course profiles of intracellular metabolites. A system of ordinary differential equations was fit based on least squares optimization to estimate purine pathway fluxes with and without radiation treatment. Post-radiation change in metabolite levels was used to estimate the dynamic fluxes with time.

Results and Discussion: Quantification of purine fluxes in mice models revealed that glioblastoma has a higher flux through IMPDH compared to the normal cortex, making it a tumor-specific target (FIG. 4A). Dynamic MFA of the purine pathway revealed that fluxes through de novo IMP synthesis and de novo GMP synthesis increase after radiation (FIG. 4B, 4C). Moreover, the ratio of de novo GMP synthesis (i.e. de novo GMP synthesis facilitated by IMPDH) flux to total GMP synthesis flux increases after radiation treatment (FIG. 4D) Taken together, these data indicate that targeting IMPDH with an IMPDH inhibitor such as MMF would be effective post-radiation in order to enhance efficacy of radiation treatment in glioblastoma.

The data from patients revealed enrichment patterns similar to mice models.

In sum, described herein is a first-principles approach to estimate metabolic flux. Specifically, this example demonstrates an approach to estimate absolute fluxes in xenograft models. The modeling described herein reveals that IMPDH can be a GBM-specific target after radiation. A similar approach can be used to characterize other pathways in different tumor types. It may also be used to characterize patient-derived xenografts to guide precision metabolic therapy.

### Example 3

The brain avidly consumes glucose to fuel neurophysiology. Cancers of the brain, such as glioblastoma (GBM), lose aspects of normal biology and gain the ability to proliferate and invade healthy tissue. How brain cancers rewire glucose utilization to fuel these processes is poorly understood. Herein, infusions of ¹³C-labeled glucose into patients and mice with brain cancer were performed to define the metabolic fates of glucose-derived carbon in tumor and cortex. By combining these measurements with quantitative metabolic flux analysis, it is demonstrated herein that human cortex funnels glucose-derived carbons towards physiologic processes including TCA cycle oxidation and neurotransmitter synthesis. In contrast, brain cancers downregulate these physiologic processes, scavenge alternative carbon sources from the environment, and instead use glucose-derived carbons to produce molecules needed for proliferation and invasion. Targeting this metabolic rewiring in mice through dietary modulation selectively alters GBM metabolism and slows tumor growth.

Gliomas are the most common form of malignant brain tumor, arising when normal glial cells of the central nervous system transform to become aggressive and invade the brain. Glioblastoma (GBM) is the most common aggressive type of brain cancer and characterized by profound invasiveness and treatment resistance. Conventional GBM treatment consists of surgical resection followed by radiation therapy (RT) and temozolomide (TMZ) chemotherapy. Despite these treatments, GBMs invariably recur, and most patients die within 1-2 years of diagnosis. Poor outcomes for patients with GBM and other forms of glioma are due largely to treatment resistance, as the extensive inter- and intratumoral genomic heterogeneity of tumors limits therapeutic efficacy. Further understanding of common targetable phenotypes in glioma could advance efforts to develop novel therapeutics and improve the effectiveness of current standard treatments. Cancer cells exhibit dramatic differences in metabolic activity relative to neighboring healthy cells and rewire the flow of metabolism to favor proliferation and treatment resistance. Moreover, in glioma altered metabolism mediates a variety of important pro-tumor processes including treatment resistance. Thus, targeting tumor metabolism for therapeutic benefit in patients is an attractive clinical strategy.

Defining metabolic pathway activity in human cancer is challenging. Positron emission tomography with glucose analogs shows high glucose uptake in both GBM and normal cortex but provides no information on how these tissues differentially utilize glucose-derived carbons. Quantifying metabolite abundance (such as through mass spectrometry-based metabolomics or magnetic resonance spectrometry) can reveal differences in metabolite levels between tumors and brain tissue. However, these steady state measurements provide minimal information regarding metabolic pathway activity, as the level of a metabolite is a function of both its rate of production and its rate of consumption. For example, high levels of a given metabolite could reflect its increased synthesis (increased activity) or its decreased consumption (decreased activity). Because these disparate biologic states are likely to respond differently to therapeutic inhibition, an understanding of the metabolic pathways that are active in a cancer would assist in appropriately guiding metabolically directed therapy.

Isotope tracing was used herein for direct interrogation of metabolic pathway activity in cancer. In this technique, metabolic substrates containing heavy (but non-radioactive) isotopes such as ¹³C and ²H are administered to living systems. Tracking these isotopes into their downstream fates by mass spectrometry or nuclear magnetic resonance-based methods yields information about which metabolic pathways are active in a system. How active other metabolic pathways are in human brain cancer and how brain cancer metabolism differs from that of cortex are unanswered questions. To answer these questions, ¹³C-labeled glucose was infused into mice bearing orthotopic GBMs and patients with high-grade gliomas, the fates of glucose carbon into numerous downstream metabolic pathways in both cancerous and cortical brain tissue was evaluated. These mass spectrometry-based measurements were paired with newly developed in vivo metabolic flux models to quantify the absolute rates of numerous metabolic reactions. The results herein demonstrate that aggressive brain cancers shift glucose carbon utilization away from physiologic processes such as TCA cycle oxidation and neurotransmitter synthesis, in part by salvaging nutrients like serine from the environment. Instead, they preferentially utilize glucose carbons to synthesize the molecules they need to grow: purines, pyrimidines, and nicotinamide cofactors. This adaptive metabolic regulation was found to be plastic, with GBMs adaptively upregulating these metabolic pathways in response to therapy. Restricting alternative carbon sources by modulating diet shifts GBM metabolism away from biomass production and slows tumor growth. Together, these studies represent the first measurements of numerous metabolic pathways in brain cancer and reveal brain cancer-specific metabolic rewiring that can be selectively targeted with dietary approaches.

### Results

¹³C Glucose Infusions into Glioma Patients and Mouse Models of GBM

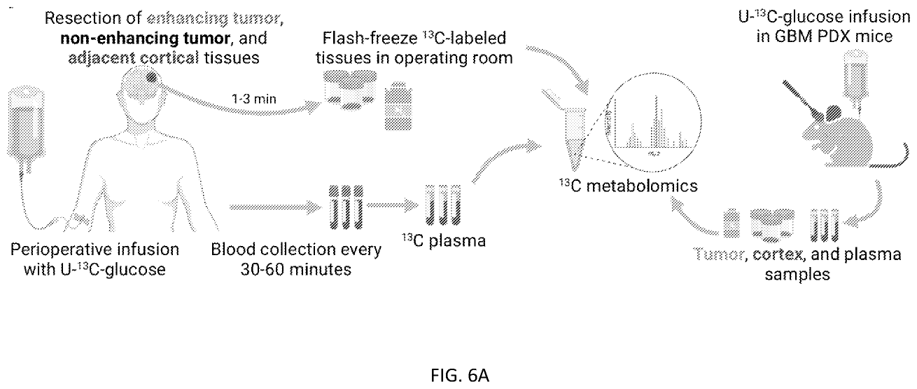

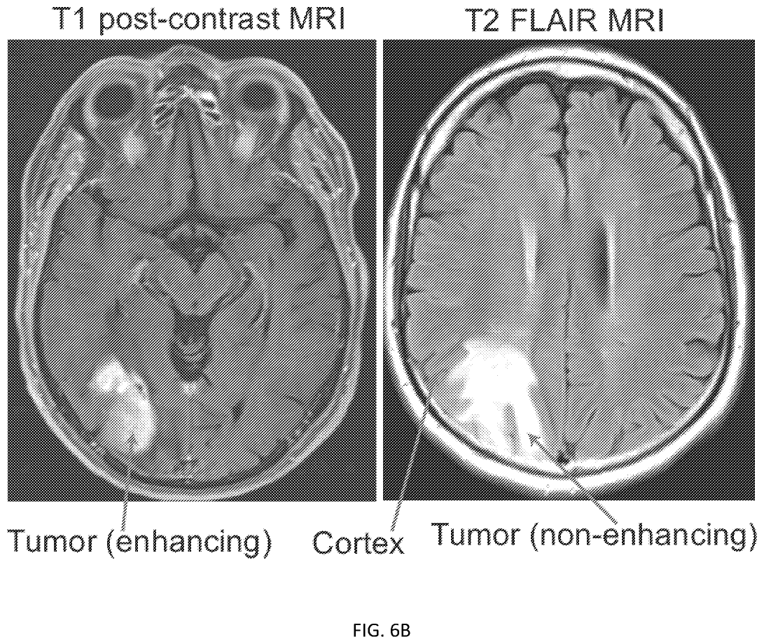

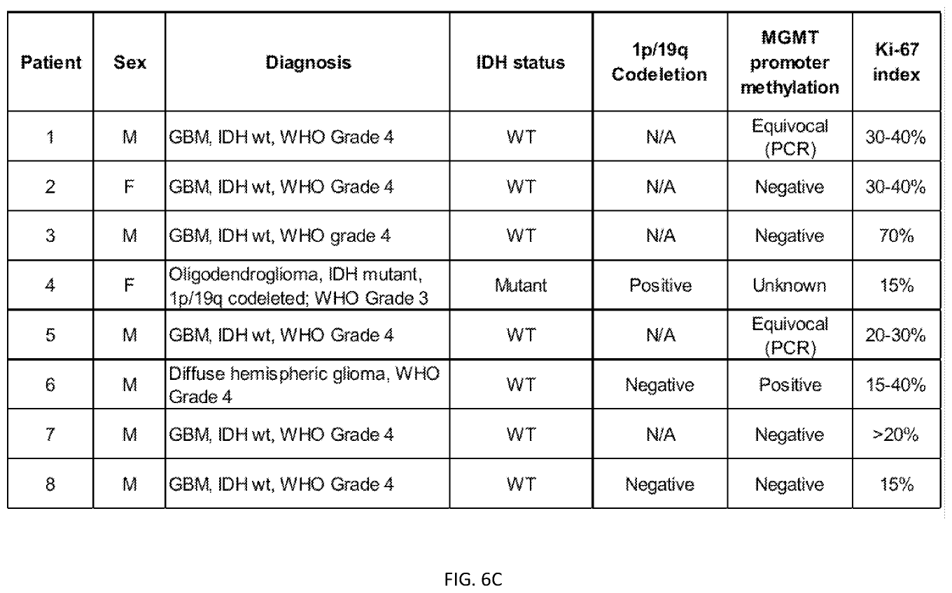

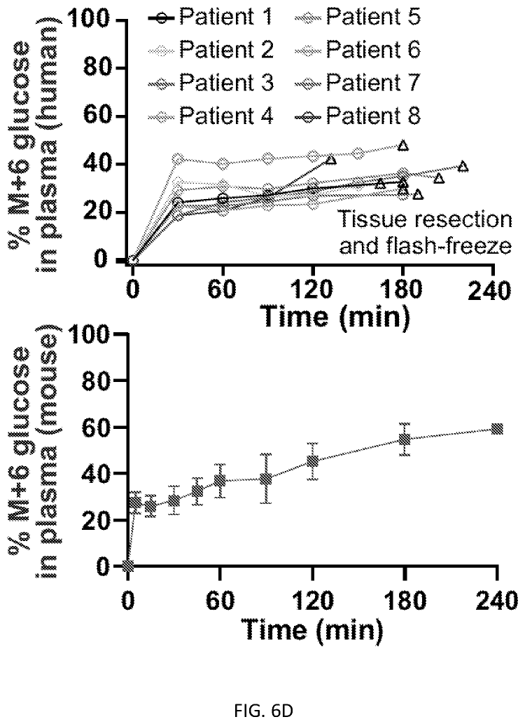

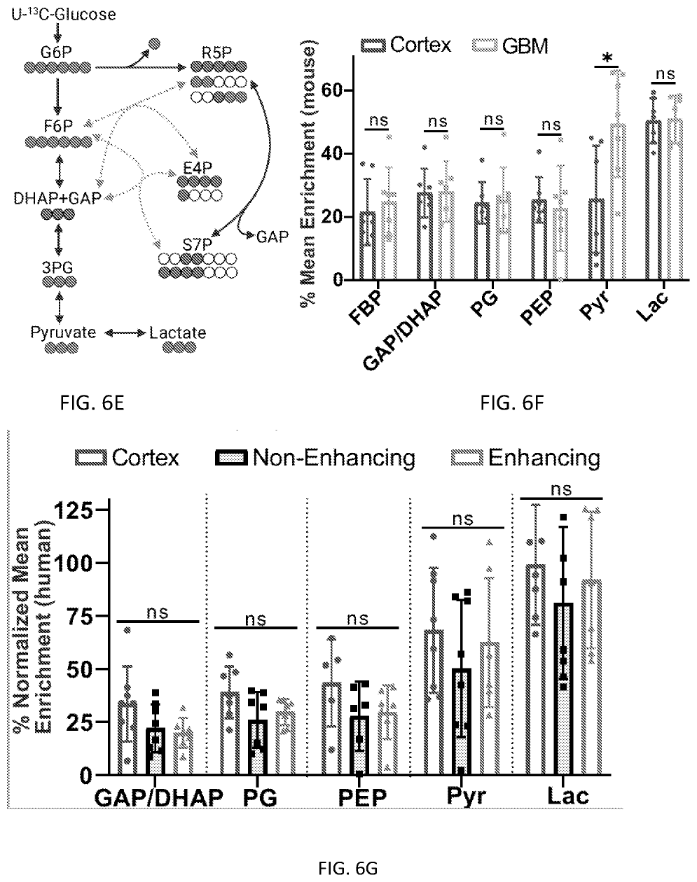

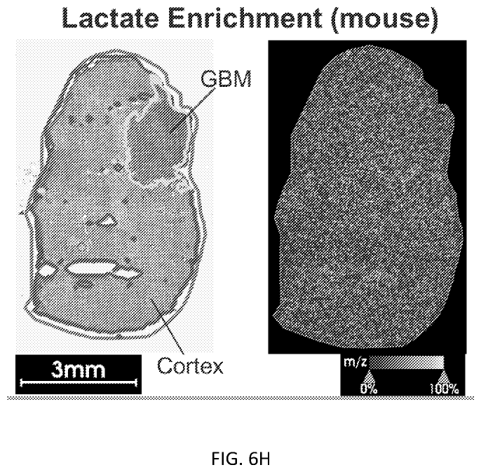

To understand the metabolic fates of glucose in brain tumors, uniformly labeled ¹³C-glucose ([U¹³C]-glucose) was infused into mice with intracranial GBM patient-derived xenografts (PDXs) and into patients with likely GBM undergoing surgical resection. Tissue was then analyzed by mass spectrometry to determine the accumulation of glucose-derived ¹³C into downstream metabolites (FIG. 6A). In mice, a treatment-resistant luciferase⁺ GFP⁺ PDX (GBM38) from the Mayo Clinic repository was used, and GFP⁺ tumors were separated from GFP⁻ cortex using fluorescent-guided microdissection. In patients, sample isolation was performed using MRI guidance by a board-certified neurosurgeon (WNA). Surgical practice for glioma patients with tumors in non-eloquent locations was to perform a supramaximal resection which removes the contrast-enhancing tumor, the non-enhancing fluid-attenuated inversion recovery (FLAIR) hyperintense tumor, and some surrounding cortex (FIG. 6B).

Eight patients were enrolled in this study (FIG. 6C), 6 of whom later were found to have GBMs, one of whom had an isocitrate dehydrogenase (IDH) mutant anaplastic oligodendroglioma, and one of whom had a histone H3 mutant G34R grade 4 glioma. Cortex and non-enhancing tumor were obtained from all 8 patients. Enhancing tumor was obtained from only 7 patients, because one tumor was comprised of entirely non-enhancing disease.

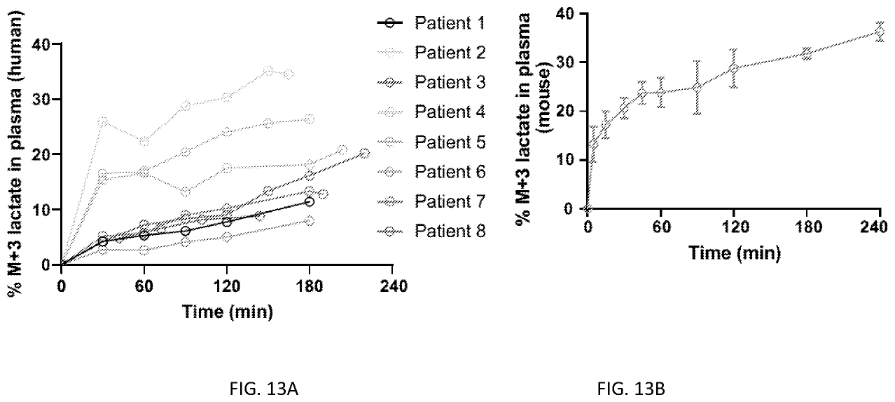

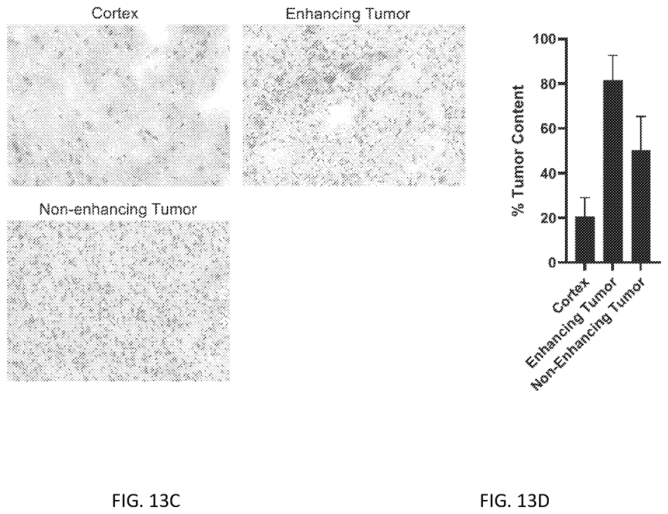

In human patients, [U¹³C]-glucose infusions lasted for the duration of the craniotomy, which was typically around 3 h. Circulating arterial [U¹³C]-glucose levels ranged between 20-40% during surgery and were typically at steady state after 30 minutes (FIG. 6D). ¹³C labeling of arterial lactate (formed from tissue conversion of infused labeled glucose into lactate, which is then secreted into the circulation) varied between patients and was typically between 10-30% (FIG. 13A). Like in patient infusions, labeled glucose reached arterial steady state in mice within 30 minutes and a total label abundance of around 50% (FIG. 6D), while ¹³C labeling of circulating lactate reached approximately 30% (FIG. 13B).

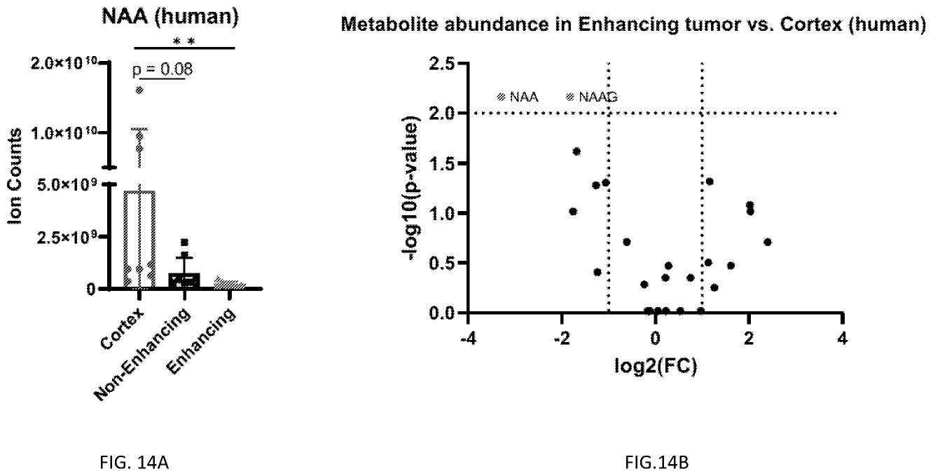

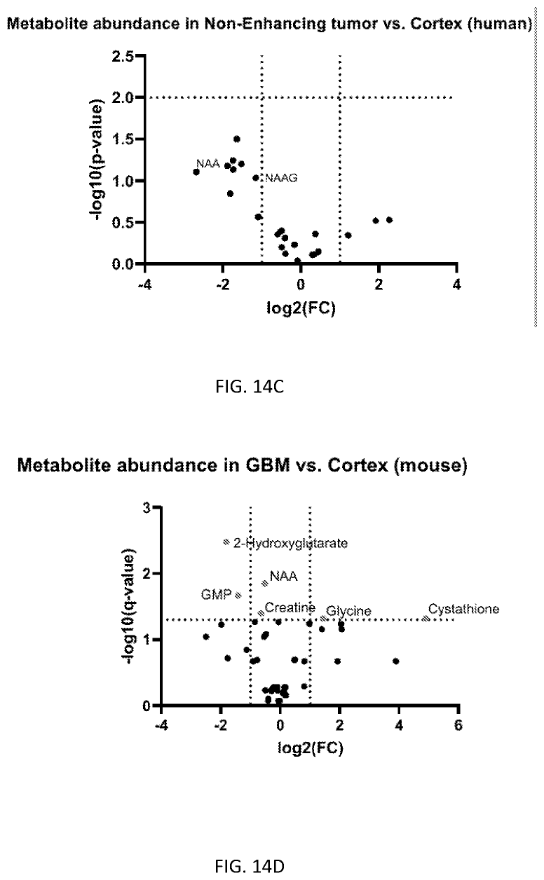

Hematoxylin and eosin (H&E) staining was used to confirm adequate separation of human samples (FIG. 13C). Tumor content quantification by a board-certified neuropathologist (S.V.) indicated approximately 80% tumor content in enhancing tumor samples, 80% cortex in cortex samples and a mixture in non-enhancing tumor samples (FIG. 13D). Consistent with these results, human cortex had nearly 10-fold higher levels of N-acetylaspartate (NAA) than enhancing tumor (FIG. 14A), thereby further supporting adequate surgical separation of the tissues. Numerous other metabolites significantly differed in absolute levels between cortex and tumor tissue in both mouse and patients (FIG. 14B-D).

Glioma and Cortex have Similar Glucose Carbon Incorporation into Glycolytic Intermediates

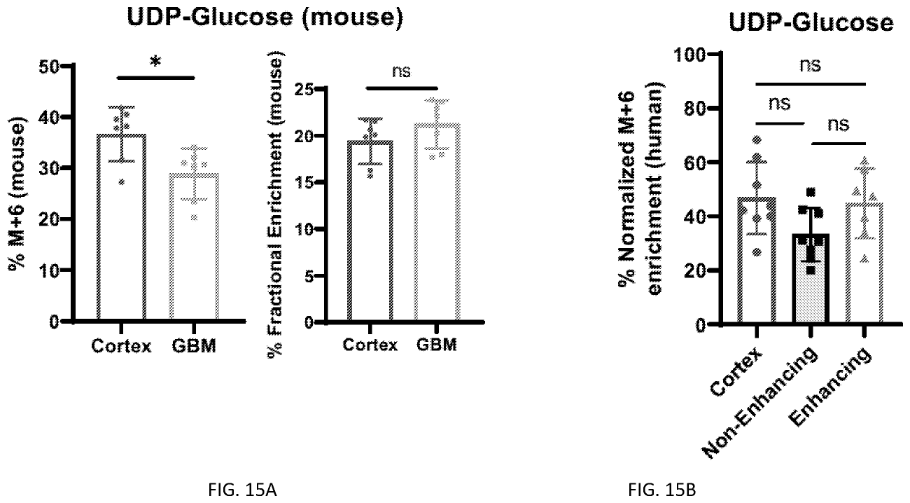

Following its entry into tissues, glucose is metabolized through glycolysis and the pentose phosphate cycle, allowing its carbons to be utilized for numerous downstream fates. ¹³C carbon incorporation into metabolites of these pathways was monitored to determine pathways active in glioma and cortical tissues (FIG. 6E). In tumor bearing mice (FIG. 6F) and humans (FIG. 6G), metabolites involved in upper glycolysis (fructose bisphosphate, GAP/DHAP, phosphoglycerate, PEP) displayed similar levels of ¹³C labeling in both GBM and cortex, indicating that labeled glucose adequately and rapidly reached both tissues. Consistent with this finding, UDP-glucose, which is rapidly formed from glucose-1-phosphate and UTP, was similarly labeled in tumor and non-tumor tissues in mice and patients (FIG. 15A,B).

Interestingly, in both GBM and cortex, the enrichment of lactate and pyruvate were higher than the enrichment of upstream glycolytic intermediates (FIG. 6F,G). These labeling patterns suggest possible ¹³C entry into lower glycolysis through lactate uptake or exchange.

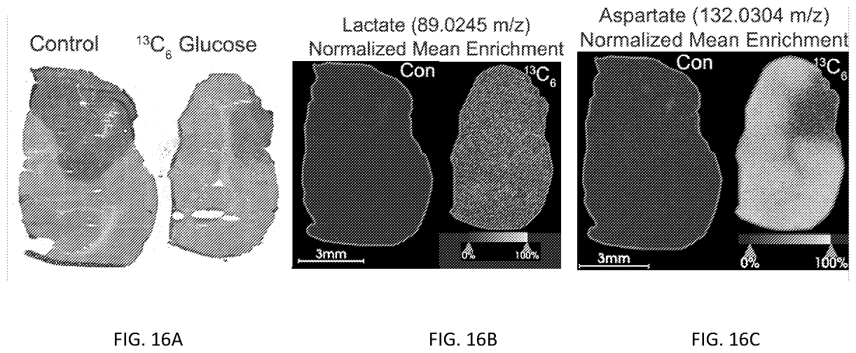

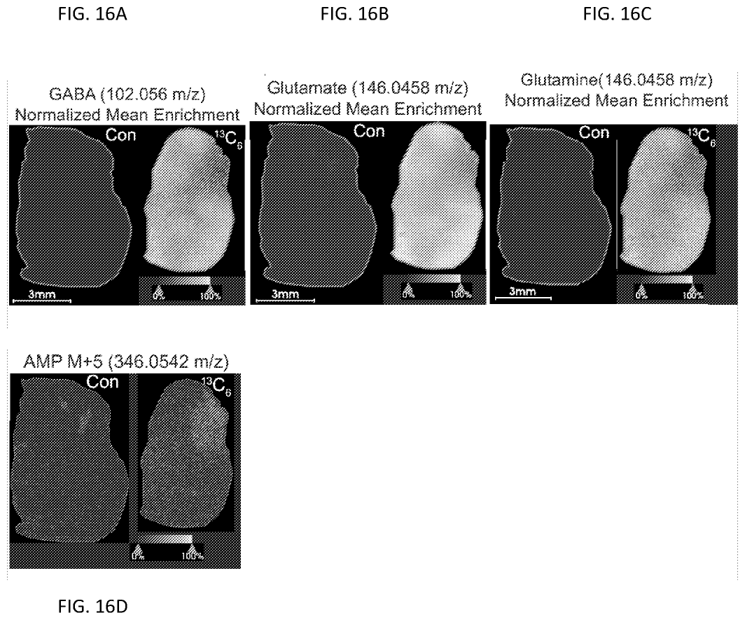

To complement these LC-MS-based analyses, slices of flash-frozen tumor tissue were also analyzed by matrix-assisted laser desorption/ionization (MALDI) mass spectrometry. This technique has the advantage of analyzing metabolite levels in non-homogenized tissue samples and thus does not require cortex/tumor separation. However, it lacks some of the sensitivity and metabolite-identification properties of LC-MS. Consistent with LC-MS results, the glycolytic product lactate had similar ¹³C enrichment in GBM tissue and cortex (FIG. 6H and FIG. 16A,B). Together, these data indicate adequate ¹³C glucose entry into tumor and cortical tissues during infusions and similar utilization/exchange of labeled extracellular lactate.

### Gliomas Reduce Glucose-Driven TCA Cycle Activity and Neurotransmitter Biosynthesis

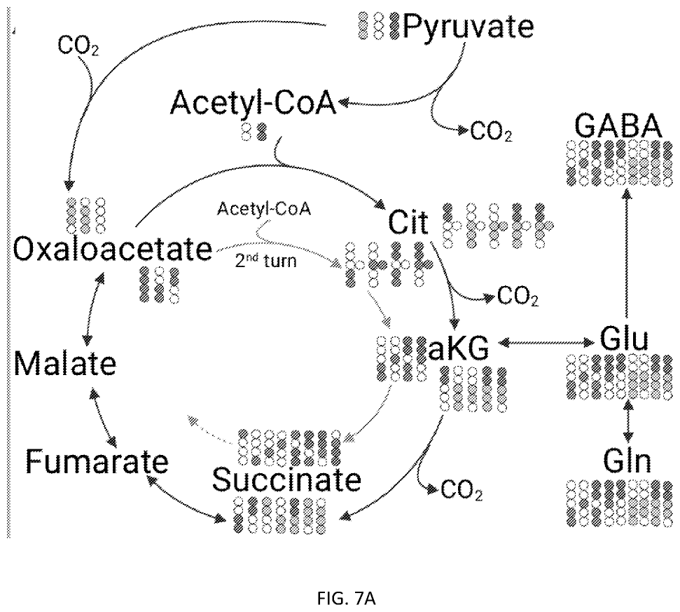

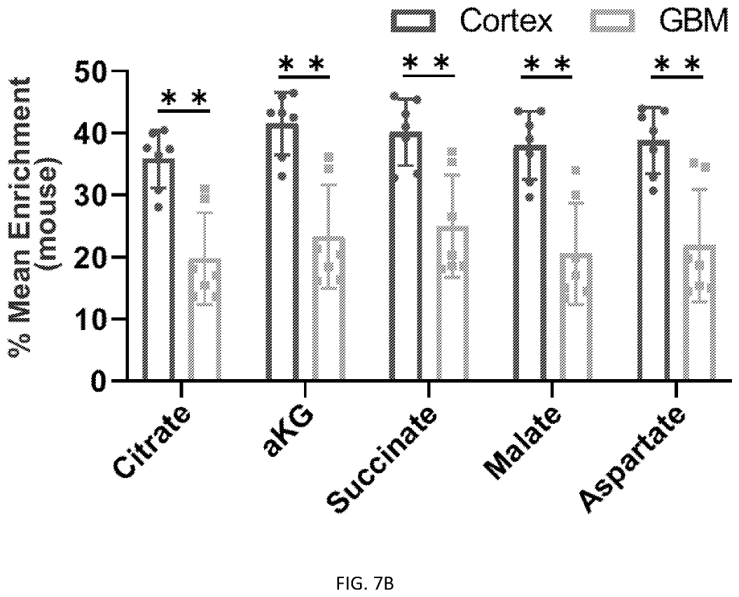

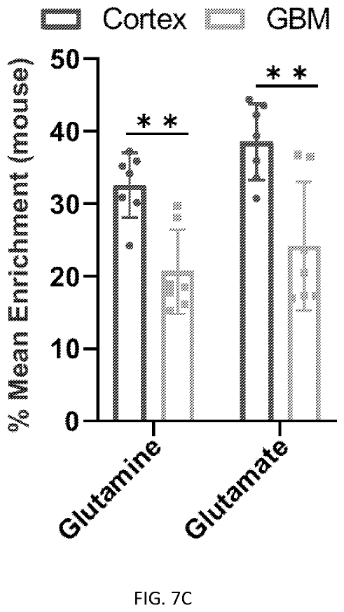

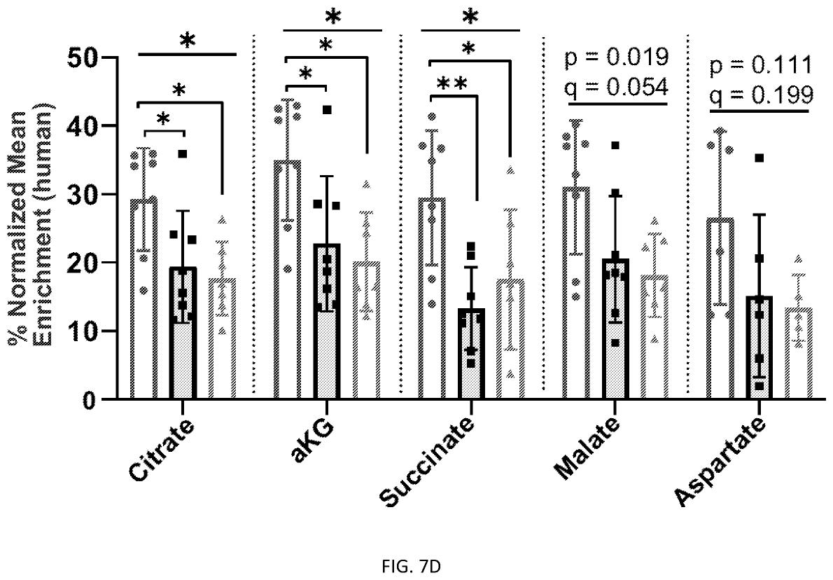

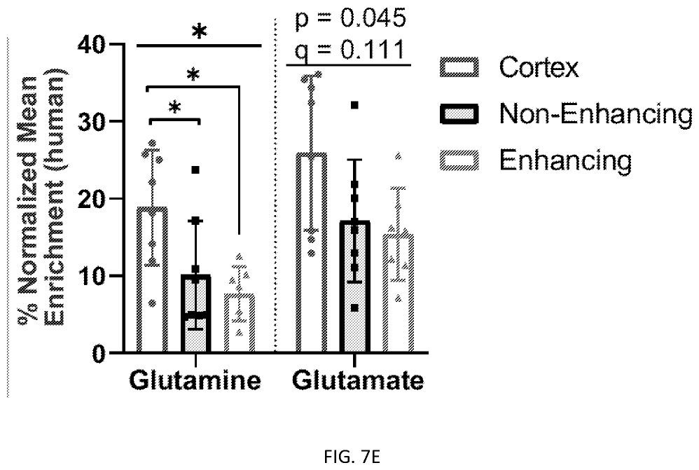

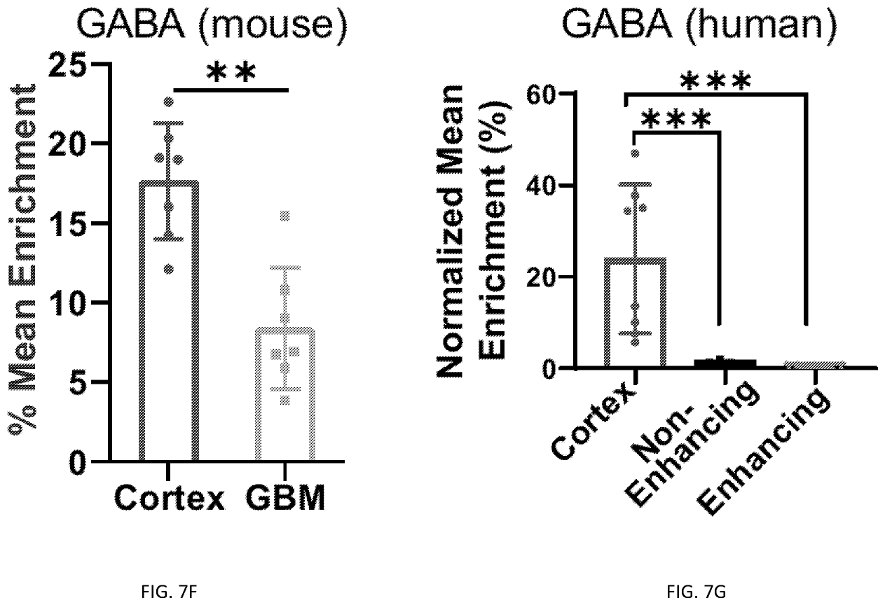

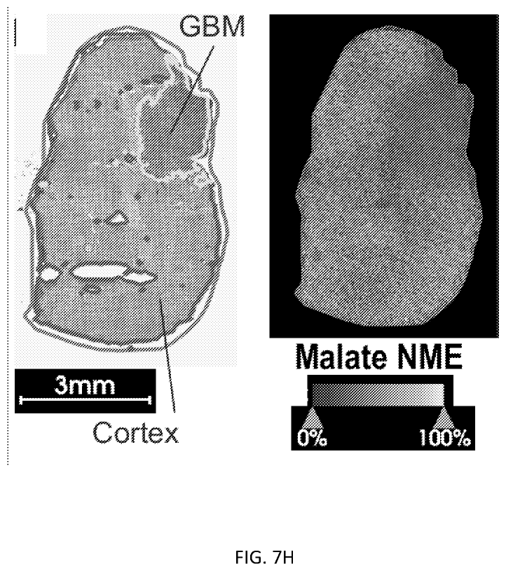

Using these same samples, metabolites in the TCA cycle, a central metabolic hub that allows for both the oxidation of glucose-derived carbons and their conversion into other molecules such as neurotransmitters and amino acids, were investigated (FIG. 7A). In mouse (FIG. 7B) and human (FIG. 7D) cortex, tracer-derived ¹³C accounted for approximately 30-40% of the TCA cycle intermediates citrate/isocitrate, α-ketoglutarate, succinate, and malate. By contrast, in tumor tissue, ¹³C accounted for only 15-20% of these TCA cycle metabolites (FIG. 7. B,D). This decrease in ¹³C labeling may indicate decreased TCA cycle activity in GBM compared to cortex and/or preferential utilization of non-glucose carbon sources to fuel the TCA cycle

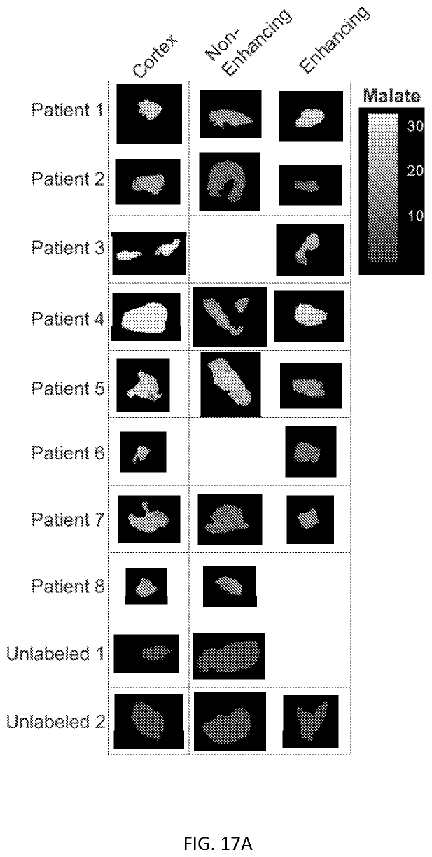

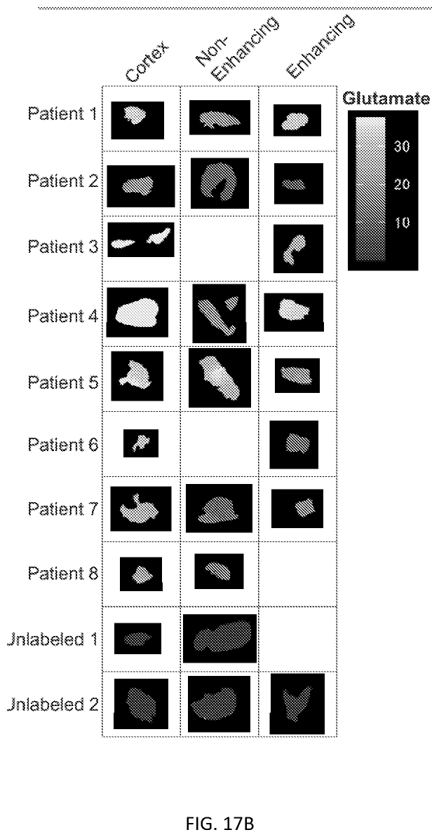

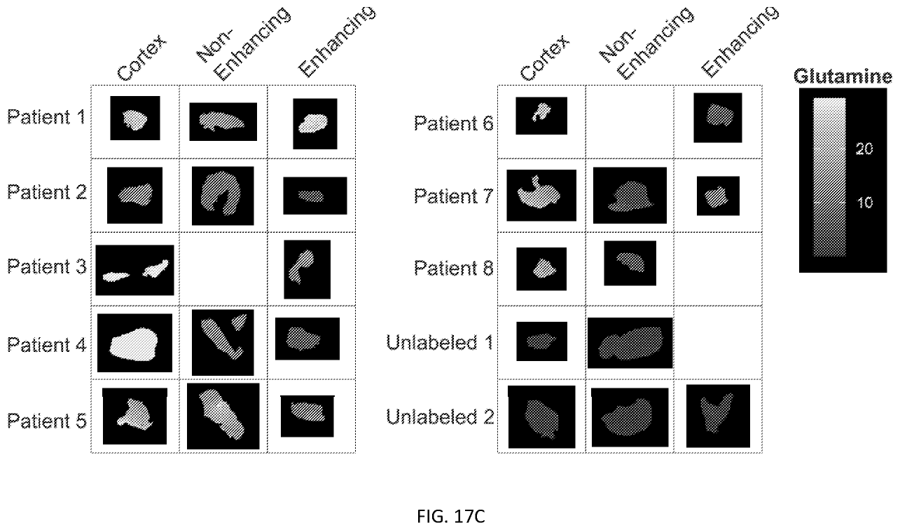

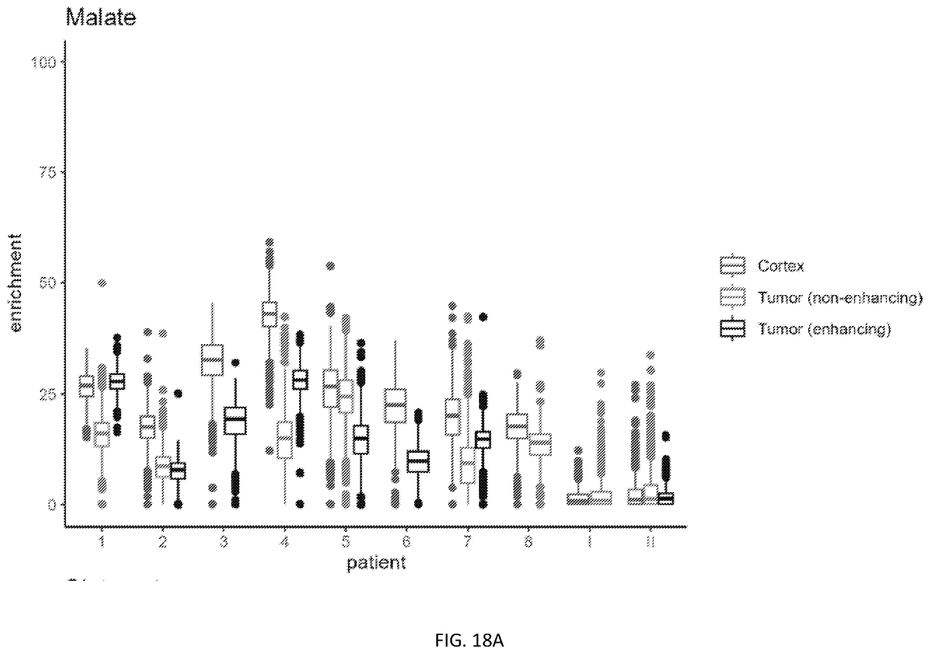

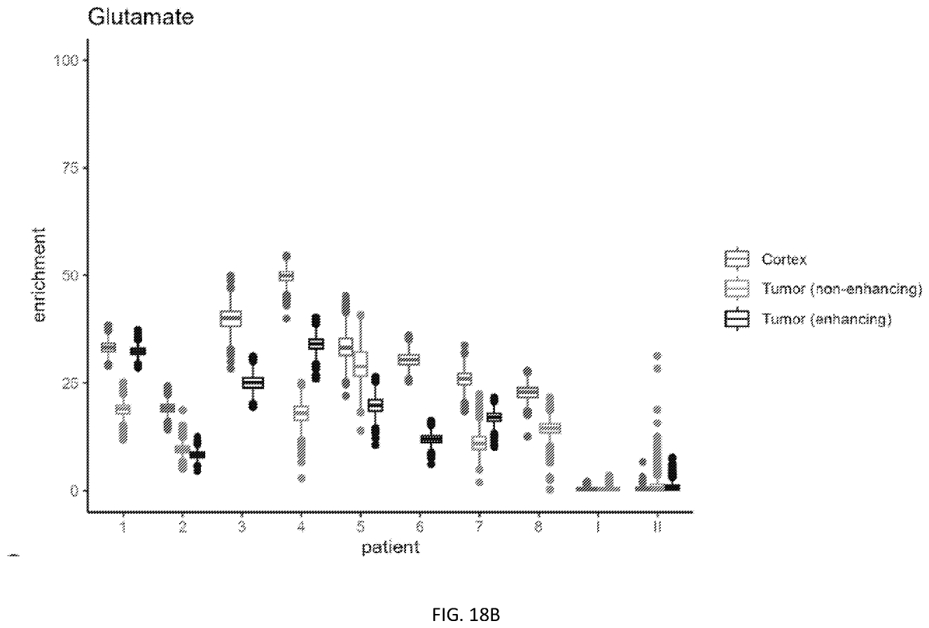

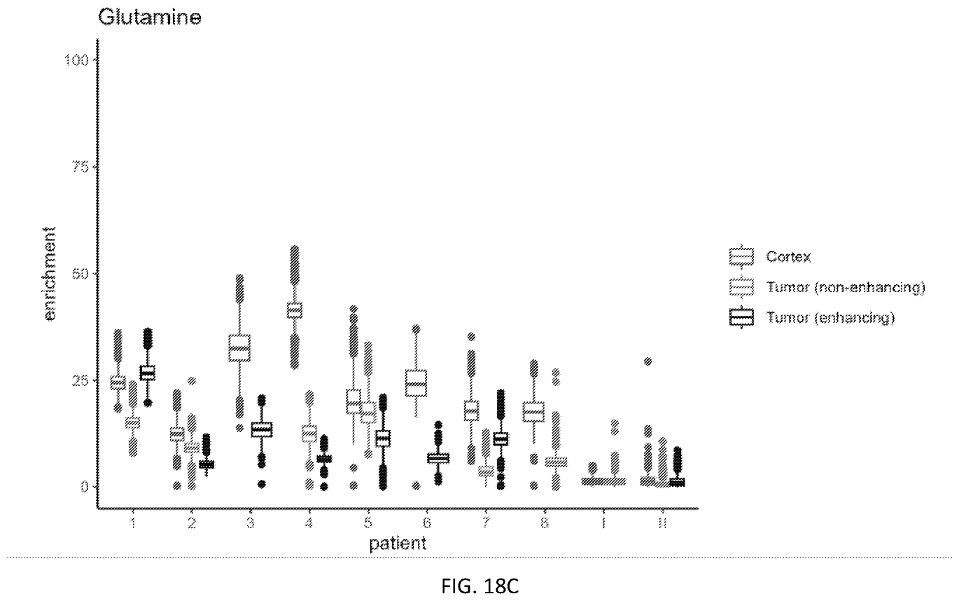

Neurotransmitter synthesis was next investigated. Glucose-derived carbons comprised a significant fraction of the neurotransmitters glutamate and gamma-aminobutyric acid (GABA) (FIG. 7C, E-G). In both human patients and mouse models, tumor tissue had lower ¹³C labeling of glutamate, glutamine, and GABA than normal cortex, with GABA labeling in human GBM virtually absent (FIG. 7G). This data demonstrates that GBMs utilize less glucose to drive neurotransmitter synthesis than does normal cortex. These measurements were largely corroborated by separate MALDI-based analysis which similarly revealed decreased ¹³C labeling in TCA cycle intermediates, glutamate, and glutamine (FIG. 7H-I, FIG. 16C-F, FIG. 17A-C, FIG. 18A-C).

Gliomas Increase Glucose Carbon Incorporation into Nucleotides

With similar glucose uptake yet lower glucose contributions to the TCA cycle and neurotransmitter synthesis, how GBMs preferentially utilized glucose-derived carbons was next determined. Labeling patterns of the glucose-derived metabolites that cells use as building blocks to synthesize the macromolecules they need to grow and divide was evaluated.

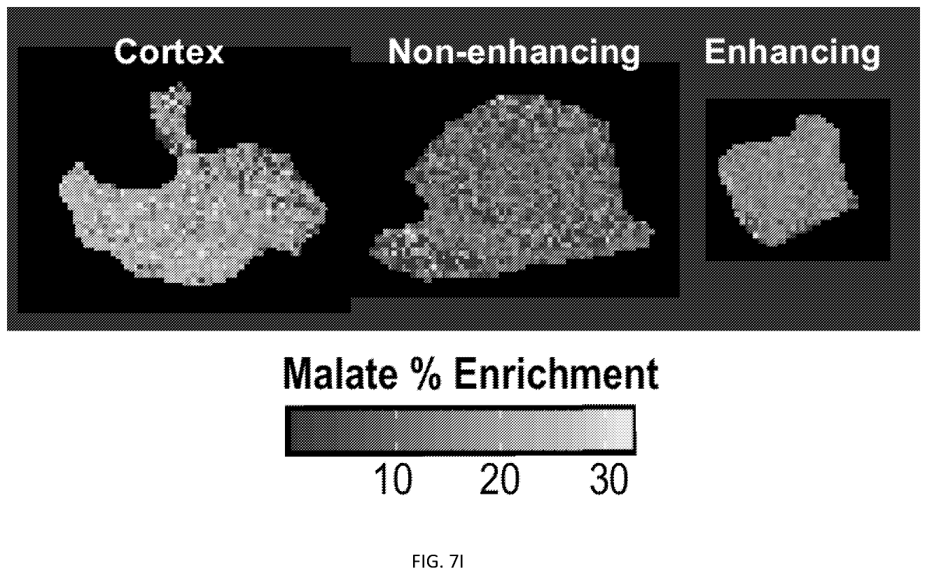

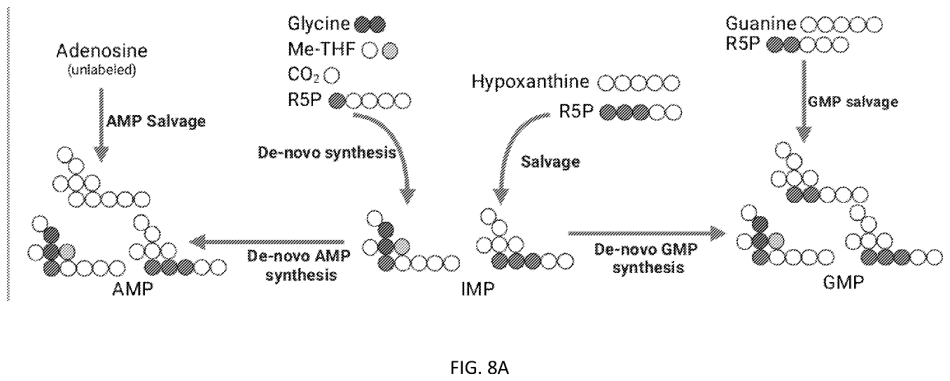

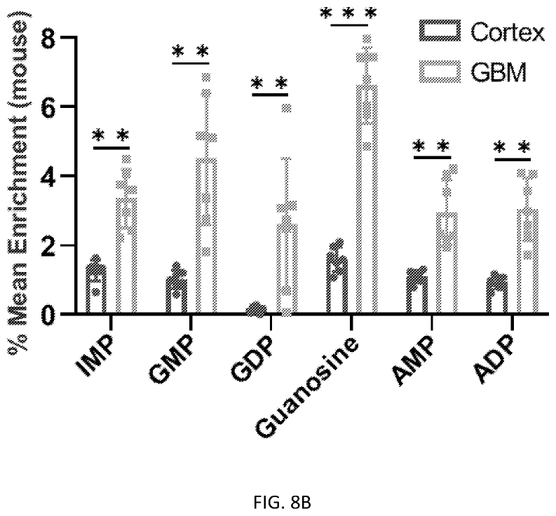

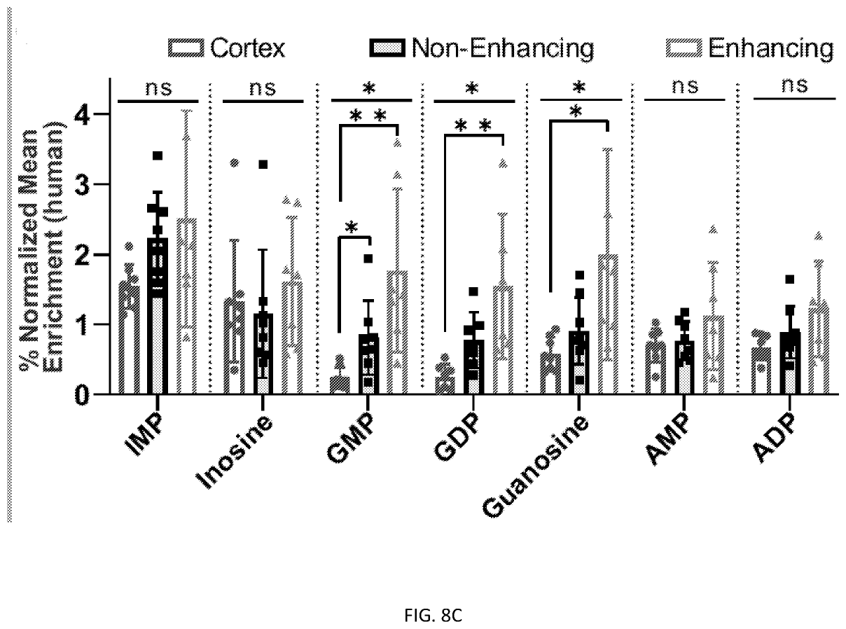

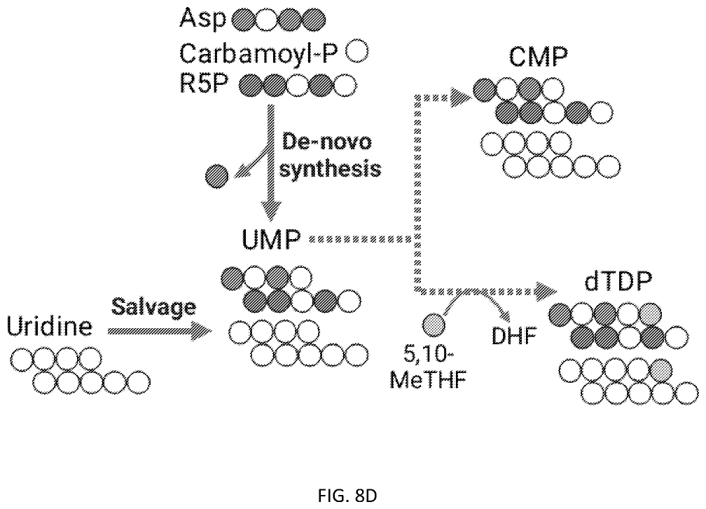

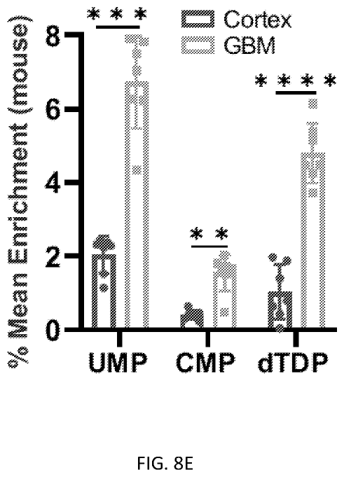

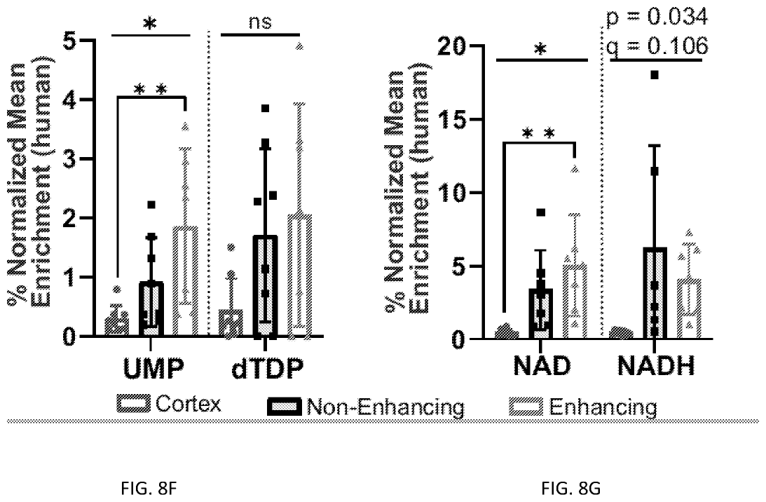

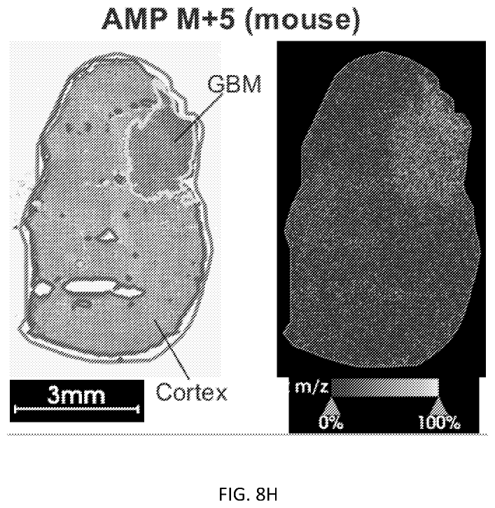

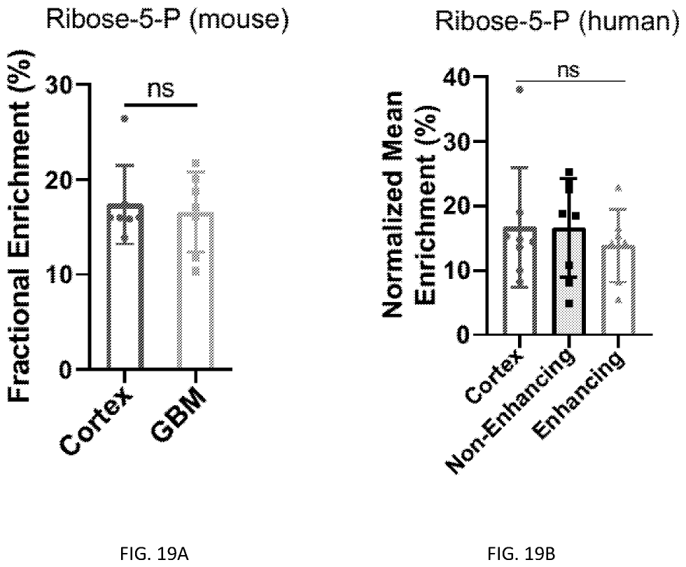

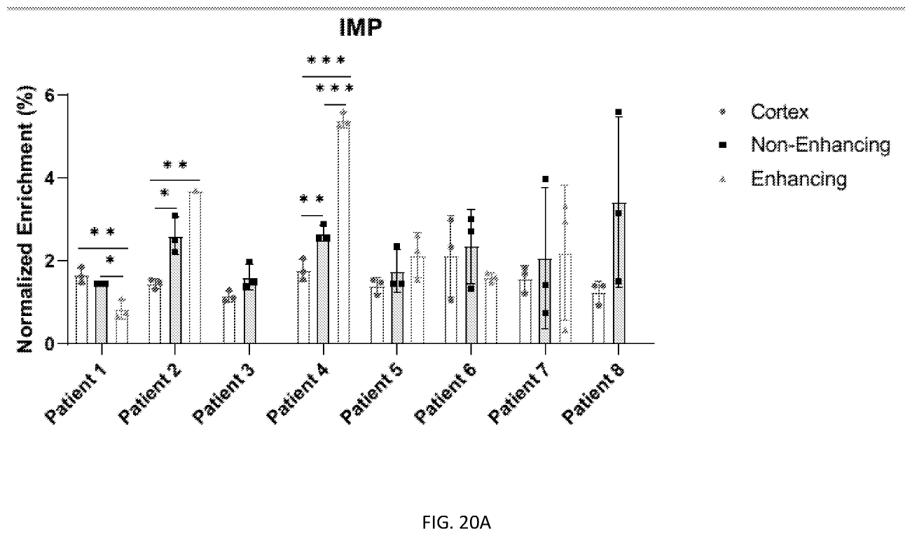

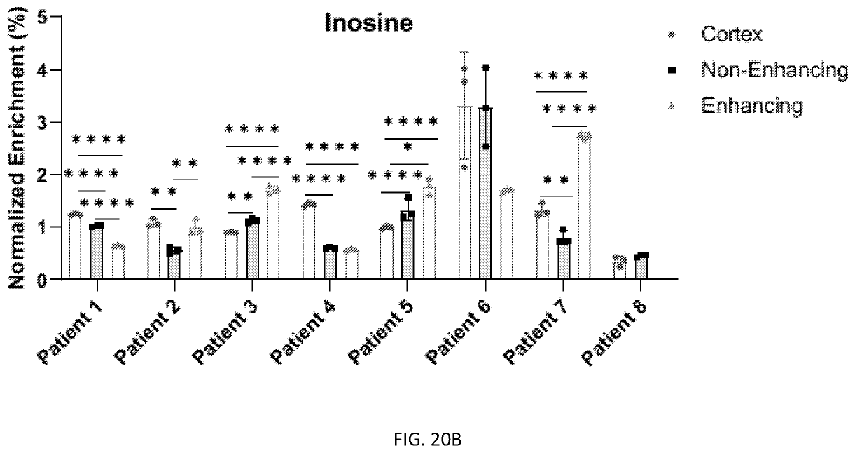

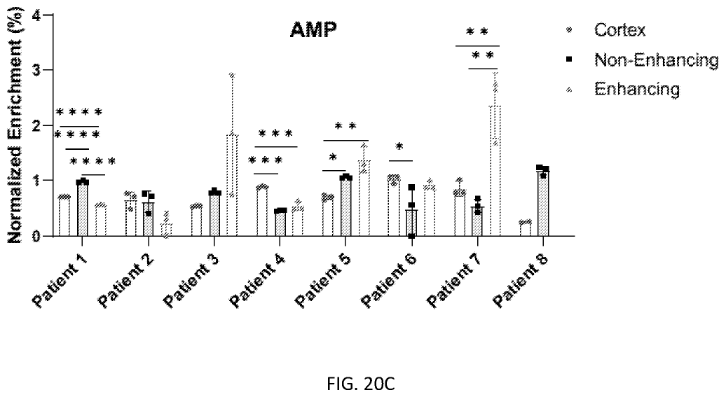

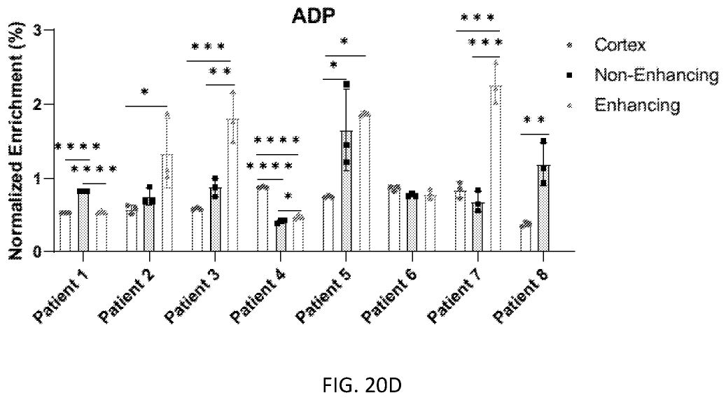

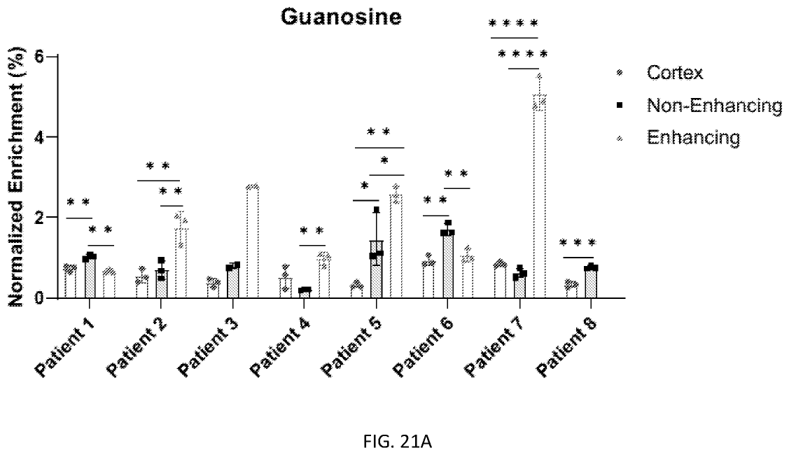

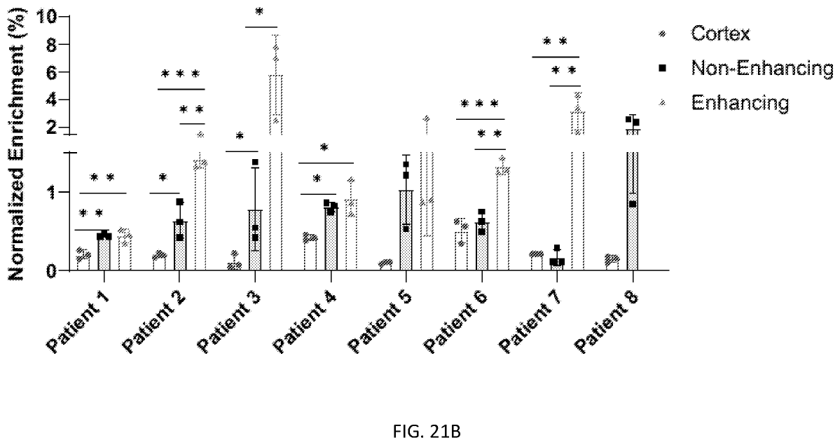

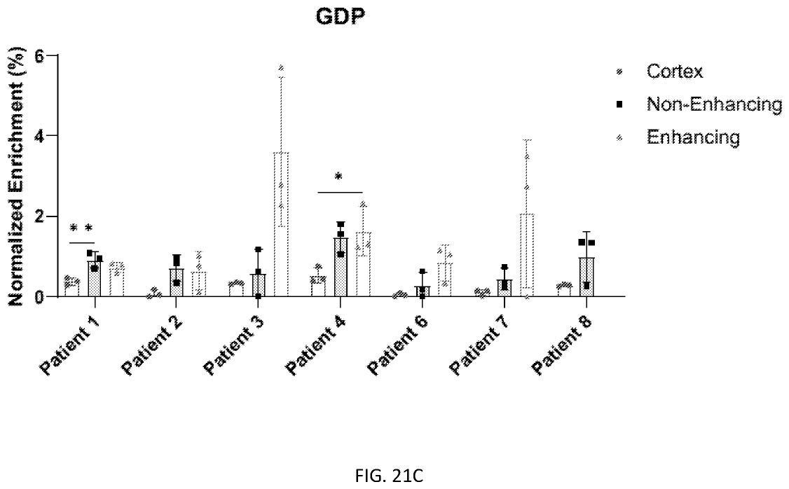

Nucleotides and their derivatives are comprised of purines and pyrimidines, which are produced through separate metabolic pathways that both use glucose-derived ribose 5-phosphate (R5P) produced in the pentose phosphate pathway (FIG. 6E). Cells may generate purines de novo, in which carbon and nitrogen from numerous sources are added to R5P in an energy-intensive process (FIG. 8A). Alternatively, cells can also salvage nucleotides by conjugating R5P with pre-formed nucleobases. (FIG. 8A). Notably, ¹³C labeling of many purine metabolites, including GMP and GDP, was increased in brain cancer relative to cortex in both mouse models (FIG. 8B) and human patients (FIG. 8C). Since the enrichment of R5P is similar between the tissues, the increase in purine ¹³C enrichment is likely a result of higher synthesis in gliomas (FIG. 19A-B). Similar results were seen using MALDI, which specifically showed increased ¹³C-labeling of purines in GBM tissue compared to normal cortex (FIG. 8H, 16G). Interestingly, when we evaluated the enrichment patterns of individual patients, we found that only the GMP arm of the purine pathway had consistently higher enrichment in all patients (FIG. 20A-D, 21A-C). This indicates that GMP production might be especially important for gliomas.

Distinct from purine synthesis, pyrimidine production is initiated by formation of a nucleobase from aspartate and carbamoyl phosphate and then conjugated to R5P to eventually yield UMP (FIG. 8D). UMP may alternatively be salvaged from uridine. UMP can be further metabolized to produce additional pyrimidines. Like purines, pyrimidines have elevated ¹³C labeling in cancerous tissues compared to cortex in both mouse models (FIG. 8E) and human patients (FIG. 8F).

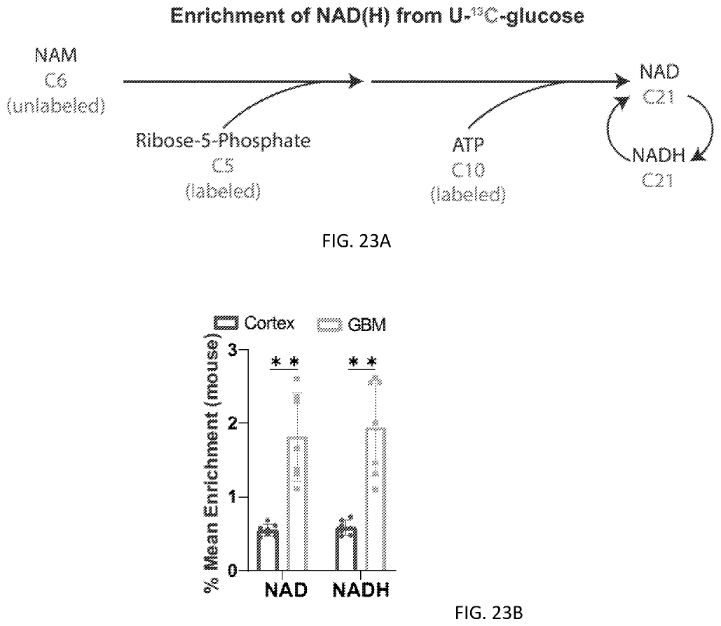

In addition to driving nucleotide synthesis, glucose-derived carbons are also used to form the essential cofactors NAD and NADH, both of which can promote oncogenic phenotypes (FIG. 23A). Consistent with findings of increased nucleotide labeling in glioma tissues compared to cortex, elevated labeling of both NAD and NADH in both mouse (FIG. 23B) and human glioma (FIG. 8G) were seen, indicating increased NAD/NADH synthesis that may support the increased growth and survival demands of tumors. Altogether these data show that gliomas rewire glucose carbon utilization away from TCA cycle oxidation and neurotransmitter synthesis and redirect them to fuel the biosynthetic needs of cancer growth.

### Gliomas Increase Flux of Nucleotide Synthesis and Decrease Oxidative TCA Cycle Flux.

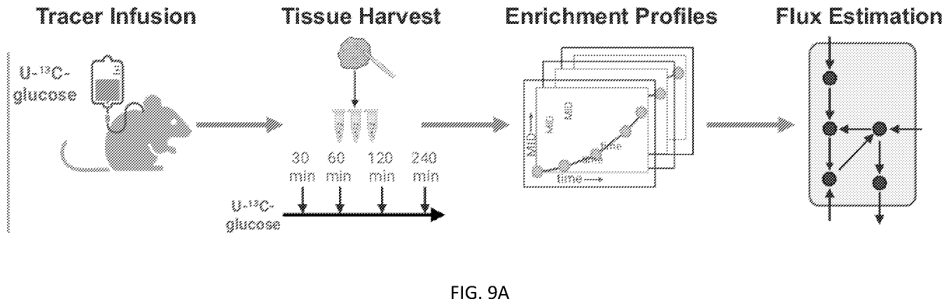

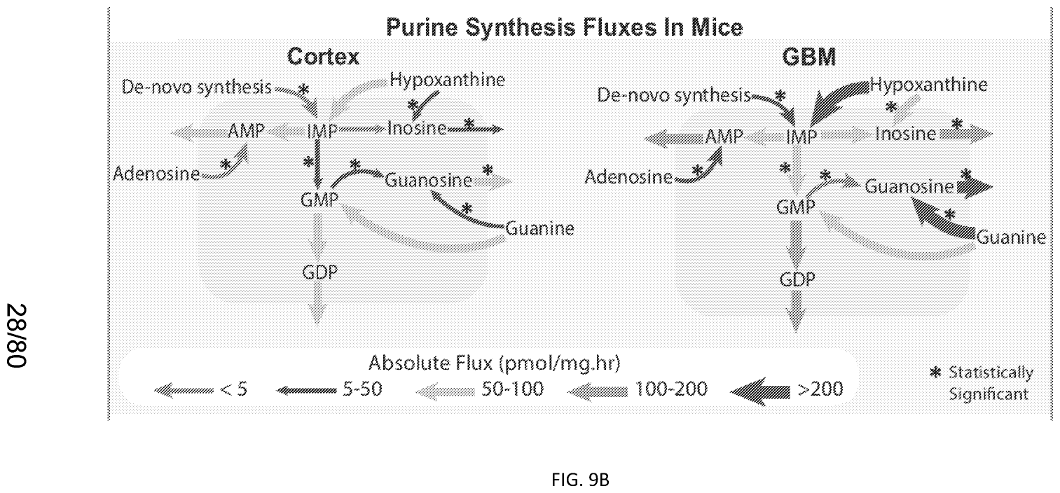

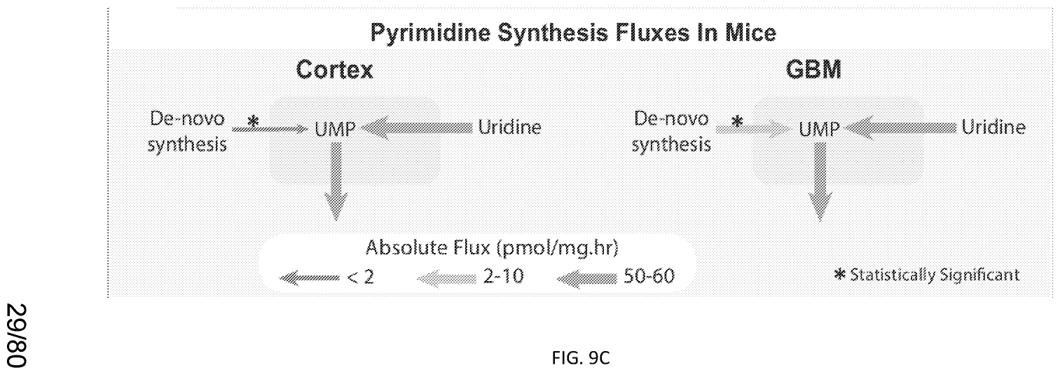

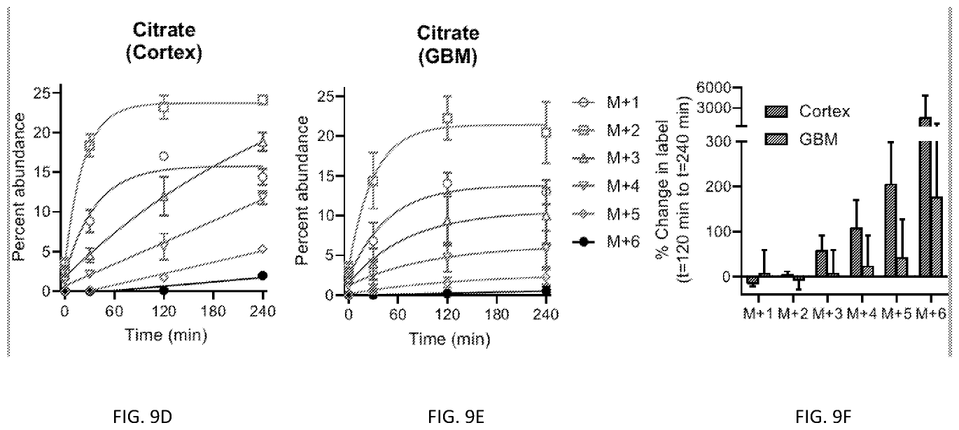

High ¹³C enrichment at a single time-point does not necessarily imply increased biosynthetic flux. For example, a nucleotide formed only from infused ¹³C-containing sources at a low rate might have higher ¹³C enrichment than a nucleotide formed from unlabeled sources at a faster rate. Therefore, a metabolic flux analysis (MFA) approach to directly quantify nucleotide synthesis fluxes from in vivo enrichment data was developed (FIG. 9A). This mathematical framework was applied to the patient-derived orthotopic GBM mouse models to determine if higher GBM nucleotide enrichment corresponds to higher synthetic flux.

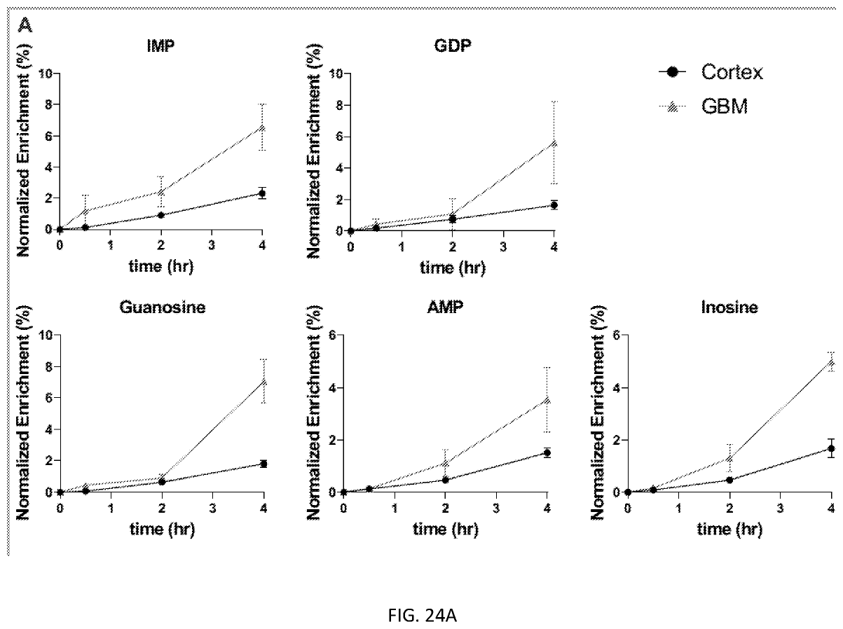

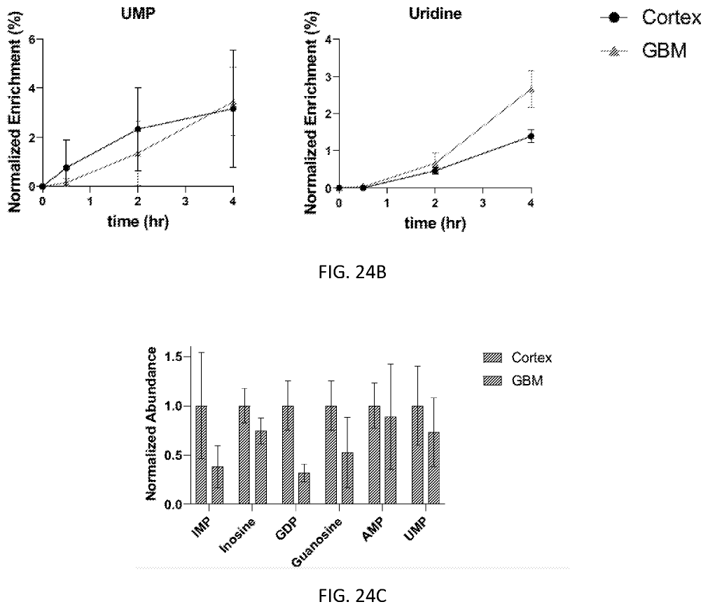

Orthotopic GBM-bearing mice were infused with [U¹³C]-glucose and GBM and normal cortical tissue were harvested for LC-MS analysis at multiple time points post-infusion to generate time-dependent enrichment profiles for MFA (FIG. 9A, 24). The time-course enrichment profiles of purine and pyrimidine nucleotides showed an increasing trend during entirety of the 4 h experiment (FIG. 24A-B), suggesting that isotopic steady state in these pathways had yet to be achieved. To overcome this limitation, an ordinary differential equation (ODE)-based MFA model using time-course nucleotide mass isotopomer distribution (MID) profiles to solve for fluxes was applied (FIG. 9A). 15 reactions in the purine synthesis pathway and 3 reactions in the pyrimidine synthesis pathway were quantified in GBM and adjacent normal cortex tissue (FIG. 25A-B, Tables 4-7).

Using MFA, significant differences in nucleotide synthesis rates between GBM and cortex were seen. In the purine synthetic pathway, GBMs have higher de novo IMP and GMP synthesis fluxes than cortex, accompanied by increased salvage synthesis of IMP and AMP (FIG. 9B, Tables 13-14). Increased synthesis of inosine, guanosine, and GDP were seen in GBM, further highlighting the broad increase in purine biosynthesis in tumors. Notably, comparison of total metabolite concentrations in GBM and cortical tissues showed that many purines were present in equal or lower abundance in GBM compared to cortex (FIG. 24C), further highlighting the increased information that can be obtained from ¹³C-MFA compared to metabolite isotope tracing and static metabolite levels-based analysis. Collectively these results indicate that the higher ¹³C enrichment of purines observed in tumors stems from higher absolute rates of purine synthesis.

Pyrimidine fluxes also differed between GBM and cortex. The de novo synthesis of UMP was elevated about 5-fold in GBM compared to cortex (FIG. 9C and Tables 14-15). However, uridine salvage was the dominant form of pyrimidine synthesis in both GBM and cortical tissue and accounted for more than 80% of UMP synthesis in both.

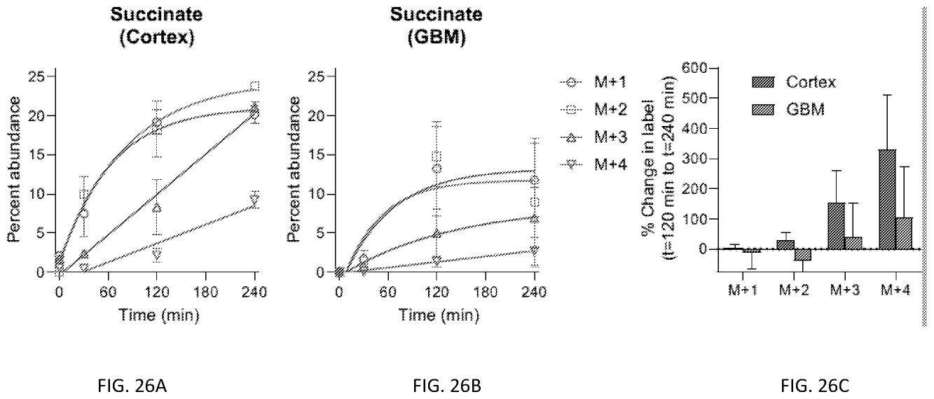

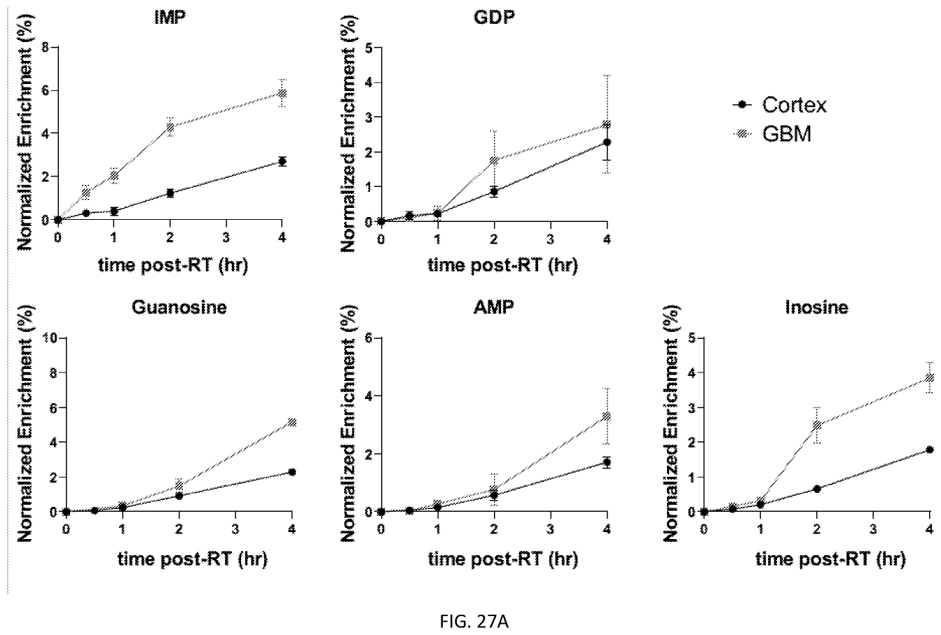

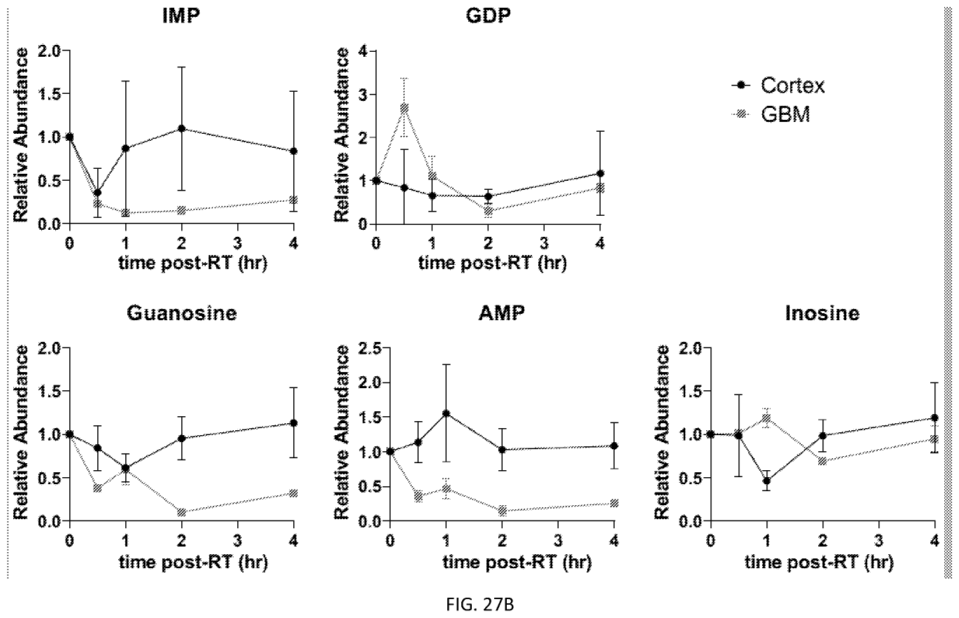

Murine time-course studies were also used to analyze TCA cycle activity in GBM and cortex. Entry of fully ¹³C₆-labeled glucose-derived pyruvate into the TCA cycle through pyruvate dehydrogenase forms TCA cycle intermediates that have two ¹³C atoms (M+2, FIG. 7A). These intermediates can then be oxidized through further turns in the TCA cycle, or they can exit the TCA cycle to drive other metabolic reactions. Intermediates that have experienced multiple turns of the TCA cycle can contain additional ¹³C atoms (M+3 to M+6, with M+3 also possible from a single turn involving the pyruvate carboxylase reaction, FIG. 7A). In both GBM and cortex, observed the rapid formation of M+2 TCA cycle intermediates (citrate, succinate and malate) over time was seen. However, the abundance of M+3 to M+6 TCA cycle intermediates steadily rose over time in cortex but remained low in GBM (FIG. 9D-E, 26A-B, D-E). Further, analyses of isotopologue distribution changes at the latest time points in these studies (t=120 min and t=240 min) in which GBM labeling approached steady state similarly revealed a consistent trend of higher labeling in cortex than GBM (FIG. 9F and FIG. 26C,F). These findings indicate higher oxidative turning of the TCA cycle in cortex and that glucose-derived TCA cycle intermediates undergo less oxidation in GBM.

### GBM Metabolic Activity is Dynamic Following Therapy

GBMs are characterized by profound treatment resistance. To determine whether metabolic adaptations facilitate the ability to respond to standard of care treatments such as RT, GBM-bearing mice were treated with cranial RT delivered to the entire brain (tumor and cortex) immediately (<5 min) prior to [U¹³C]-glucose infusion and harvested tumor and cortex over time as in FIG. 9.

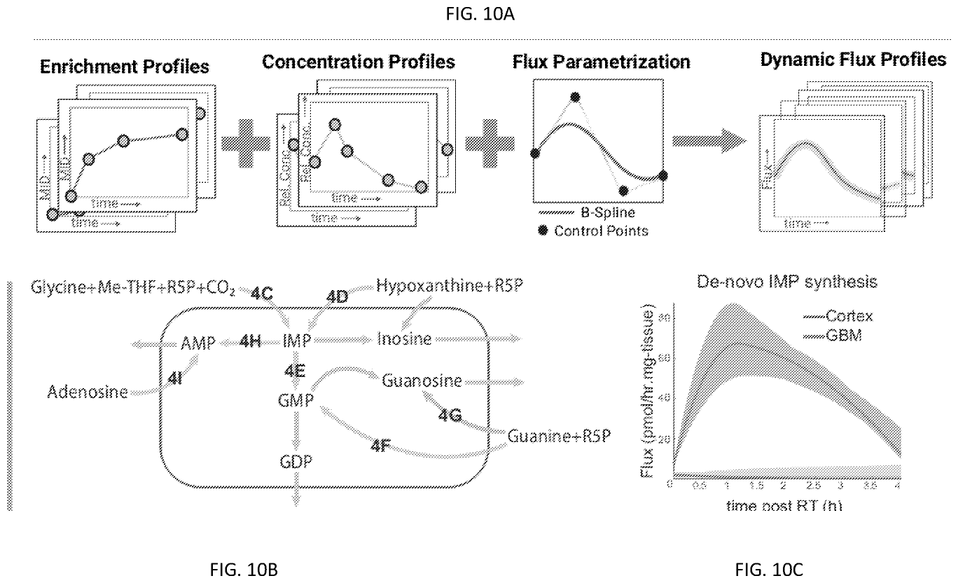

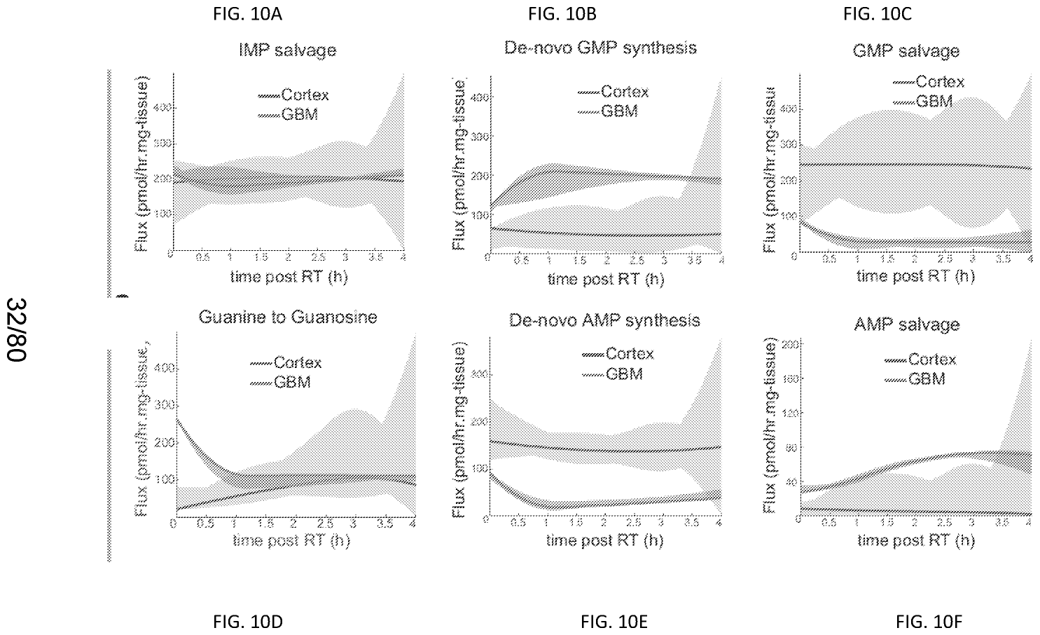

Initial MFA models (FIG. 9) assume metabolic steady state throughout the infusion. This assumption is unlikely to be true after RT due to the rapid activation of the DNA damage response and subsequent cell cycle arrest. Therefore, a dynamic-¹³C-MFA (DMFA) model to quantify purine synthesis fluxes after RT was developed (FIG. 10A).

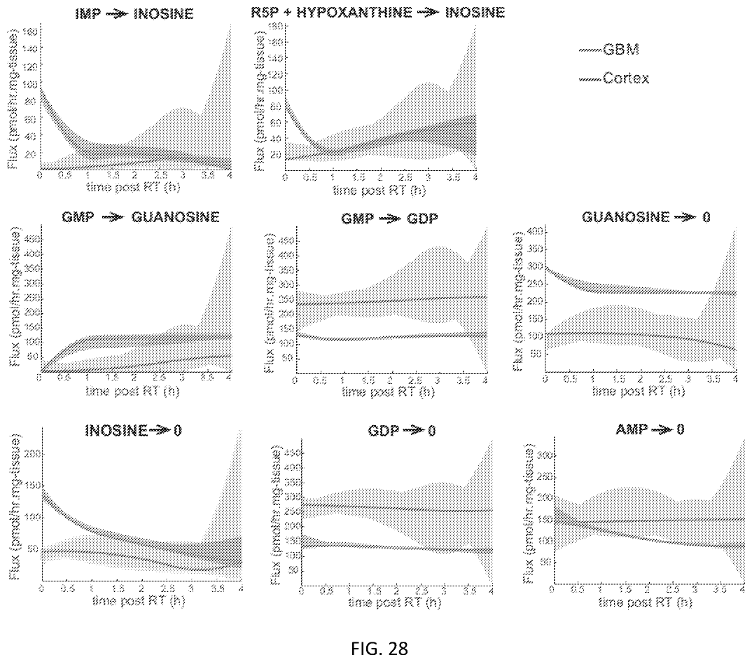

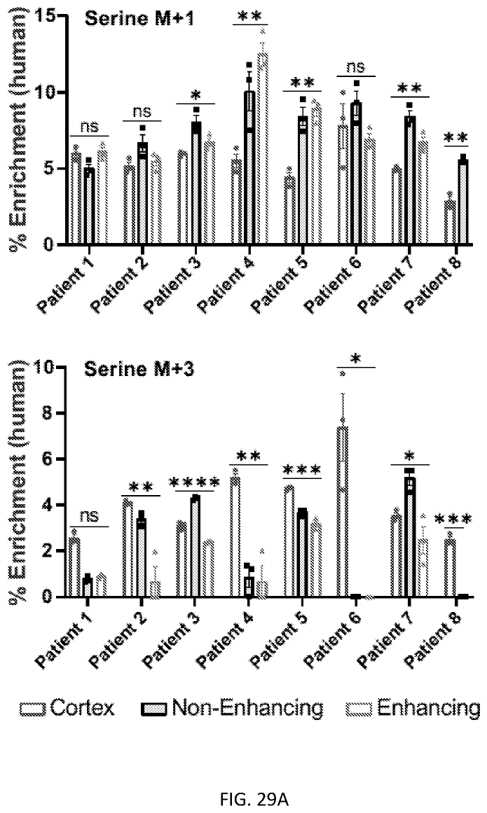

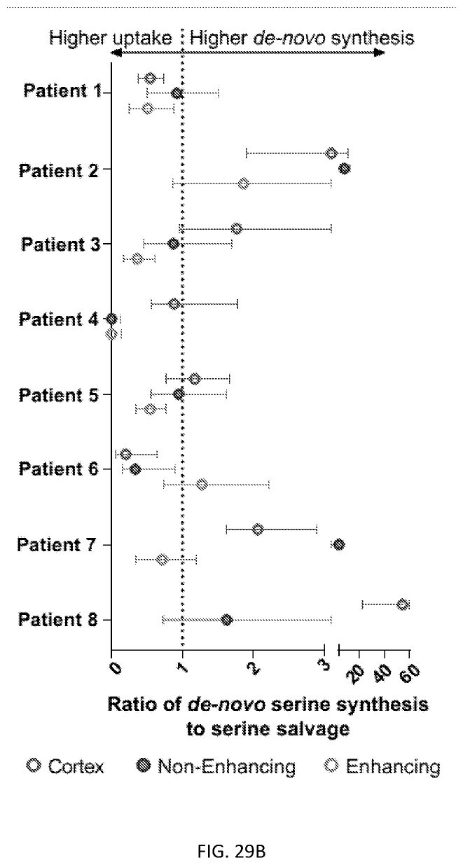

A DMFA model incorporates time-dependent concentration changes to estimate transient flux profiles. The in vivo ¹³C-MFA framework was modified to include dynamic concentration and flux changes (FIG. 10A, 27A-B, Supplemental Methods). Strikingly, purine fluxes changed dynamically after RT in GBM but remained largely unaffected in cortical tissue (FIG. 10B-I, FIG. 28). De novo IMP synthesis increased transiently after RT, with peak activity at approximately 1 h post-RT and diminishing over the next 3 h (FIG. 10C). Notably, this pattern and timeframe of increased de novo IMP synthesis is well-aligned with the known timeframe of DNA damage and repair following RT. In contrast, IMP salvage from hypoxanthine was unaffected in both GBM and cortex (FIG. 10D). De novo synthesis of GMP from IMP also increased over approximately 1 h, consistent with increased IMP synthesis, and this increase was sustained for the next 3 h (FIG. 10E), while guanylate salvage and de novo AMP synthesis both decreased after RT (FIG. 10F-H). Increase in AMP salvage partially compensates for lower de novo AMP synthesis (FIG. 10I). Together these data indicate that after RT, GBMs acutely increase de novo IMP synthesis that feeds into increased guanylate production with an accompanying decrease in AMP production.

### Gliomas Preferentially Rely on Environmental Serine

Synthesis of purines via the de novo pathway requires a variety of amino acid substrates including serine, which is a neurotransmitter, a driver of lipid synthesis and a precursor for glycine and one-carbon units. Because serine metabolism is important for a variety of tumors including GBM, it was next investigated whether isotope infusions could help us understand serine metabolism in human brain cancer.

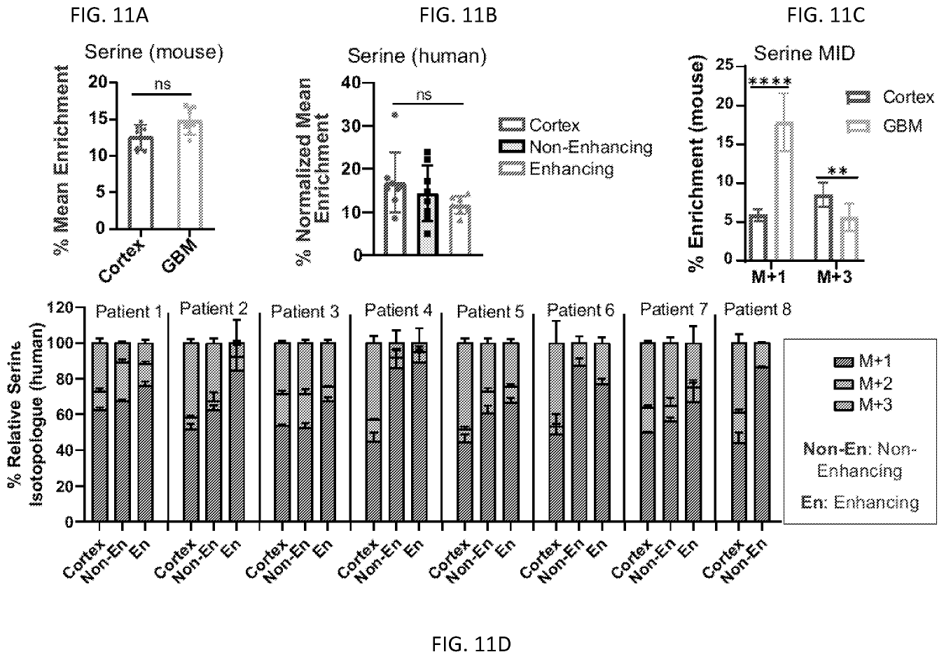

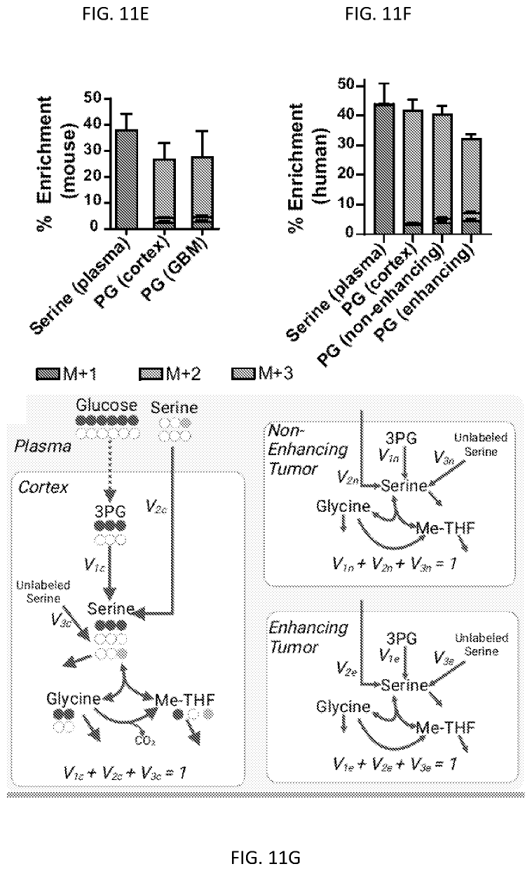

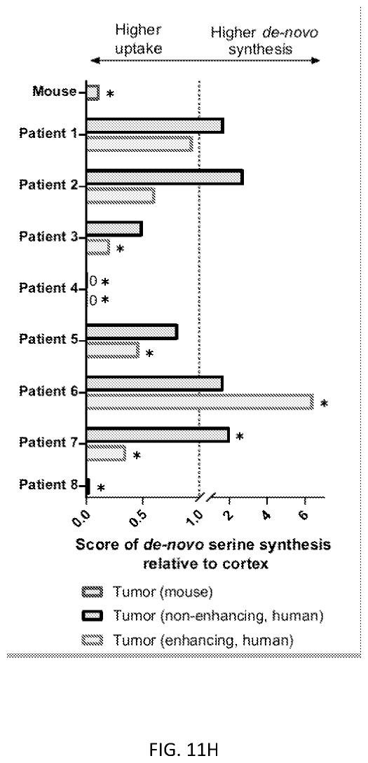

In both murine (FIG. 11A) and human (FIG. 11B) studies, total ¹³C labeling of serine was similar in brain cancer and cortex samples. This finding was distinct from many other amino acids, neurotransmitters and nucleotides, which exhibited differential label enrichment in brain cancer and cortex (FIG. 7, 8). Deeper investigation of labeling patterns in murine GBM and cortical tissue reveal that M+3 labeling of serine predominated in cortex while M+1 labeling predominated in GBM tissue (FIG. 11C). Similar labeling patterns were observed in most human tissues, in which M+3 serine was higher in cortex than enhancing tumor in nearly every case, while M+1 serine predominated in both enhancing and non-enhancing tumor tissue in most patients (FIG. 11D, 29A).

Labeled serine has multiple potential sources including (1) de novo synthesis from glucose, (2) uptake from the environment, and (3) synthesis from the addition of a carbon from the folate cycle with the two-carbon amino acid glycine. In both cortex and GBM tissue in mice, the glycolytic intermediate phosphoglycerate (PG), which is the precursor of serine, is almost exclusively M+3 labelled in both cortex and GBM (FIG. 11E). Like in mice, human PG is predominantly M+3 labelled in all tissues (FIG. 11F). Thus, any serine formed de novo from glycolytic intermediates would also be predominantly M+3 labelled. The predominance of M+3 serine in cortex and M+1 serine in brain cancer suggested that cortex predominantly generates its serine from glucose while GBM tissue likely relied on other sources.

Potential alternative serine sources for brain cancer were investigated. Arterial serine in both patients and mice was predominantly M+1 labelled (FIG. 11E, F), likely due to synthesis from unlabelled glycine and a labelled folate carbon from the kidney and liver. Thus, higher reliance on uptake of circulating serine could give rise to the M+1 serine seen in murine and human brain cancer samples. However, another potential source of M+1 is reverse SHMT flux, which can produce M+1 serine from the combination of unlabelled glycine and labelled 1-C units in the folate cycle (FIG. 11G). Given this complexity, a multicompartment ¹³C-MFA model was developed to understand the relative magnitudes of these fluxes in the patient tumors (FIG. 11H). The model was comprised of multiple tissue compartments, with all compartments having the same source of circulating serine (see methods). To ensure uniformity in the results, the external serine synthesis/uptake in all the compartments was constrained to 1. The resulting score of de novo serine synthesis flux to serine uptake flux is depicted in FIG. 11H (calculation of the score is described in methods). A ratio higher than 1 would signify a higher contribution of de novo serine synthesis in the tumor relative to the cortex, while a ratio lower than 1 would mean higher reliance on serine uptake relative to the cortex. In mouse samples, cortex predominantly relied on de novo serine synthesis while GBM samples derived most serine from extracellular sources (FIG. 11H). There was some heterogeneity in patient samples. While cortex predominantly generated serine de novo, many enhancing (6 of 7) and non-enhancing (4 of 8) tumor samples primarily relied on extracellular serine uptake (FIG. 11H, 29B). Together, these data indicated that the relative flux of de novo serine synthesis is lower in many brain cancers compared to the cortex, and that brain cancers scavenge serine from the environment.

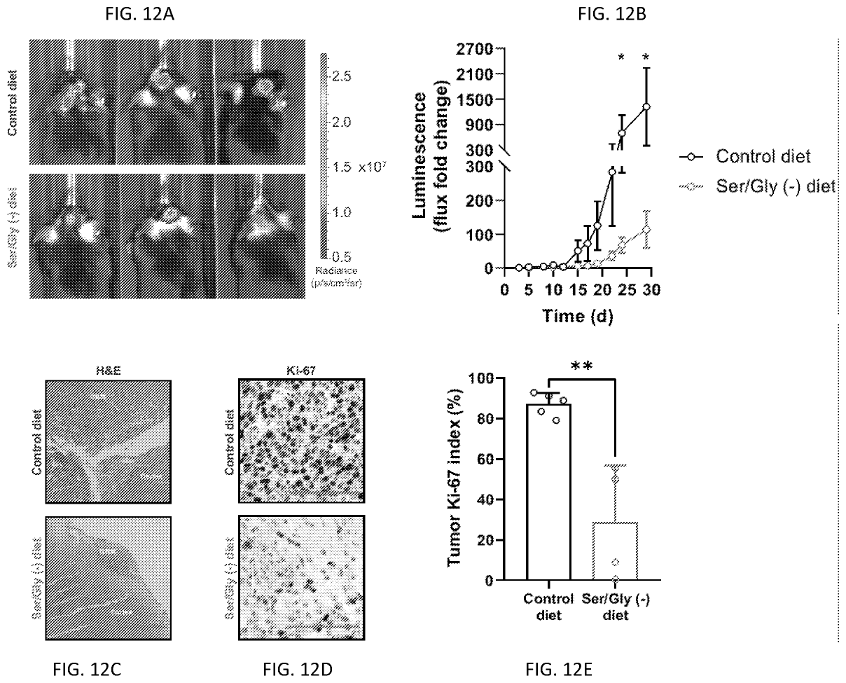

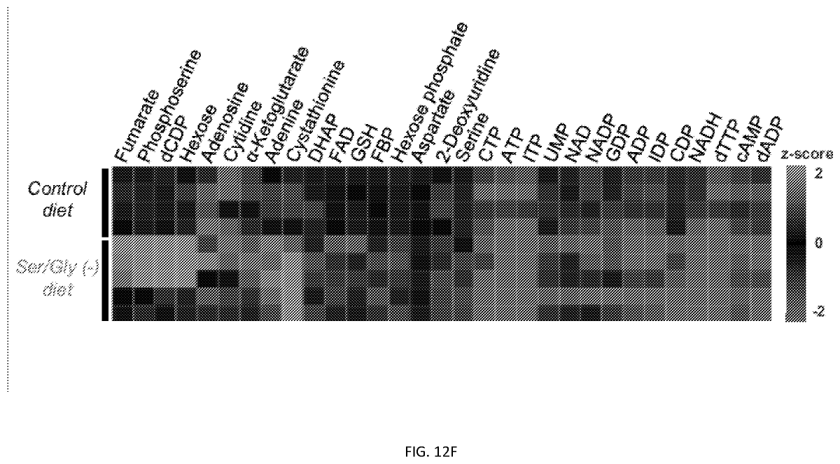

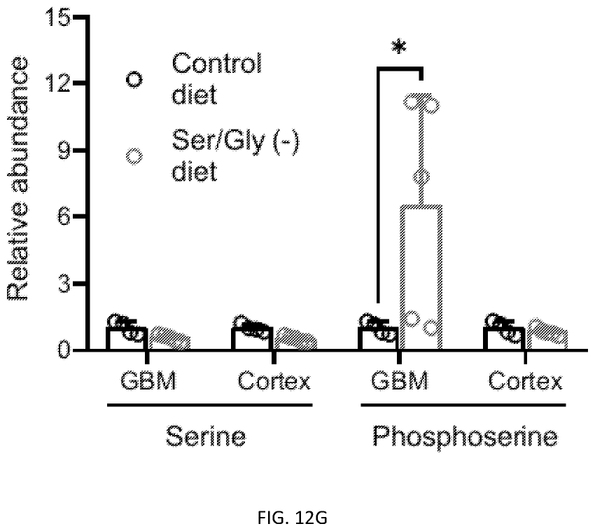

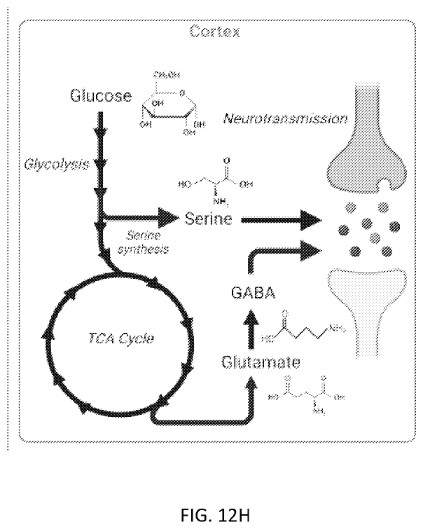

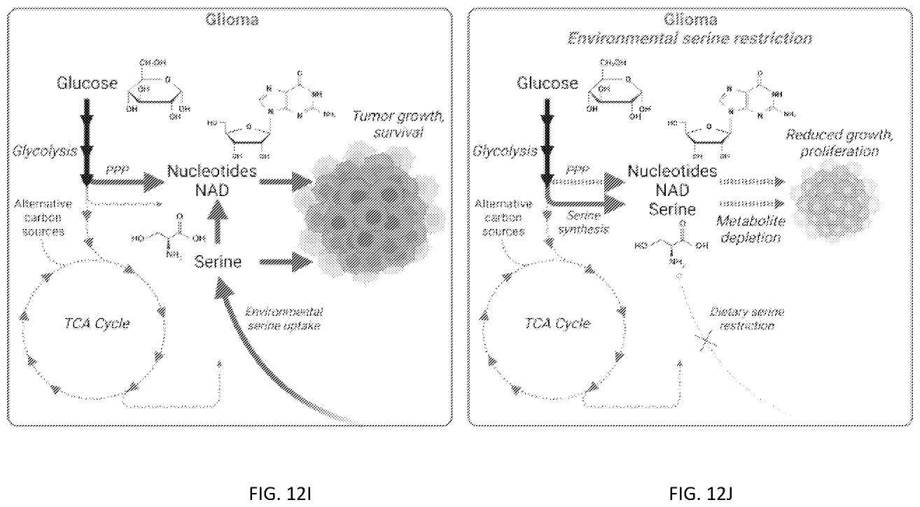

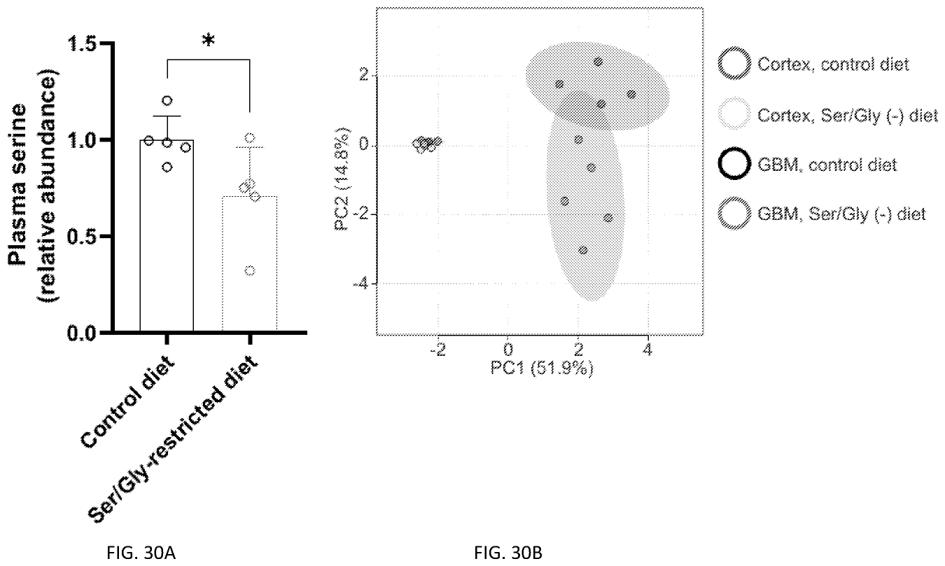

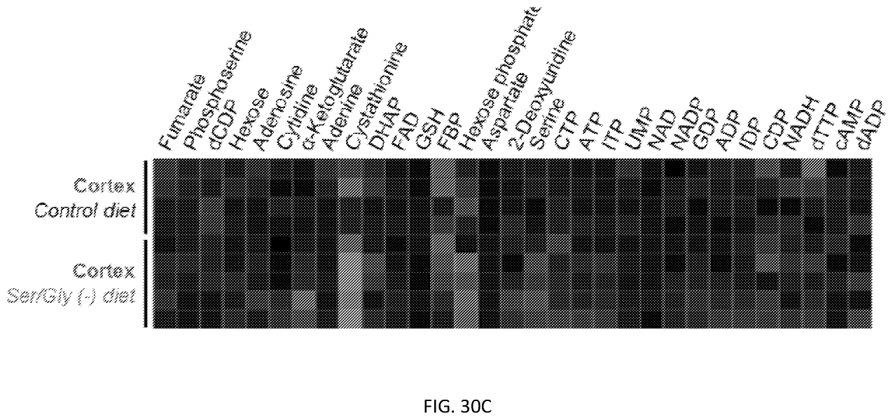

This observation suggested that brain cancers shift towards serine uptake and downregulate glucose-driven serine synthesis so that they can instead utilize glucose carbon for biosynthesis and growth. To test this hypothesis, environmental serine was restricted in orthotopic GFP⁺ fluc⁺ GBM-bearing mice by feeding them a serine-restricted diet (FIG. 12). Dietary serine restriction decreased circulating serine levels as expected (FIG. 30A) and significantly slowed tumor growth as assessed by bioluminescence (FIG. 12A,B). When control mice neared a humane endpoint, mice were euthanized and brain and tumor tissues were harvested from all groups for analysis. Tumors in serine-restricted mice were smaller than controls (FIG. 12C) and had a lower proliferation index as measured by Ki-67 staining (FIG. 12D-E). Metabolite quantification of showed that the serine/glycine restricted diet dramatically altered the metabolome of GBM tissues as assessed by principal component analysis but had little effect on cortical metabolism (FIG. 30B, 30C). This observation is consistent with glucose-driven de novo serine synthesis being the predominant route of synthesis in cortex. GBM samples from mice on low serine/glycine diets had lower nucleotides, NAD⁺ and NADH compared to GBM samples (FIG. 12F) but only modestly decreased serine levels (FIG. 12G). These findings suggested a compensatory re-routing of glucose carbons towards serine within the tumor when extracellular serine sources were limiting. Consistent with this hypothesis, phosphoserine levels (an intermediate formed when glucose carbons are shunted towards serine) were dramatically elevated in GBM tissue from mice fed a serine/glycine restricted diet (FIG. 12G). Together these data indicate that many brain cancers preferentially rely on extracellular sources for serine but can adapt to serine restriction by slowing proliferation and rerouting glucose carbons away from biomass production and towards serine synthesis (FIG. 12H-J).

In summary, experiments herein reveal a profound rewiring of carbon metabolism in aggressive human brain cancers that fuels tumor growth. These cancer-specific metabolic alterations, such as the preference for environmental serine and the reliance on IMPDH to synthesize GTP, are potential therapeutic targets with favorable therapeutic indices.

### DISCUSSION

Herein, a clinical stable isotope tracing program was developed and tested which integrated ¹³C based infusions with ¹³C-MFA to define the metabolic rewiring that occurs in brain cancer and understand its therapeutic implications. While both brain cancer and cortex avidly consume glucose and engage in glycolysis, cortex predominantly utilizes glucose-derived carbons for physiologic processes such as TCA cycle oxidation and neurotransmitter synthesis. Brain cancers downregulate these physiologic processes and instead use glucose-derived carbons to synthesize nucleotides including NAD⁺ and NADH, which they use to fuel proliferation. By developing quantitative ¹³C-MFA models, upregulated de novo purine and pyrimidine synthesis in GBM and a robust GBM-specific metabolic response to radiation therapy was identified. Finally, it is demonstrated herein that cortex synthesizes a higher fraction of its serine from glucose, while brain cancers salvage serine from the environment. This metabolic rewiring is a targetable liability with a therapeutic window. In mouse models, dietary serine restriction depletes GBM nucleotide pools and slows tumor growth while minimally affecting the metabolism of the cortex.

This work provides insights into how metabolism is altered in human brain cancers and adds to a growing body of knowledge regarding metabolic rewiring in cancer. Infused [U¹³C]-glucose rapidly accumulates in both GBM and cortex. The findings herein suggest that broadly targeting glucose uptake in attempt to slow GBM growth is unlikely to have a favorable therapeutic window and may lead to untoward toxicity. An active TCA cycle in brain cancer was observed. However, the unique surgical practice herein allowed for the first comparisons of metabolic pathway activity in human cortex and brain cancer to be completed. While the TCA cycle is active in GBM, it appears downregulated compared to non-malignant cortex with both a preference for non-glucose substrates and decreased turning of the cycle. Routes of serine synthesis have not been interrogated in human cancer. Unlike brain metastases, which appear to rely on de novo serine synthesis in preclinical models, GBMs preferentially rely on environmental serine, and do so to allow glucose carbon to be used for nucleotide synthesis and proliferation instead.

The ¹³C-MFA models described herein provide insights into metabolic rewiring that are difficult to glean with simpler analysis techniques. While de novo synthesis of both purines and pyrimidines is elevated in GBM compared to cortex, salvage of uridine and hypoxanthine appears to dominate nucleotide production. This situation appears to differ from that of other brain tumors, where the de novo synthesis of pyrimidines is dominant. These results suggest that targeting the upstream steps of de novo nucleotide synthesis in GBM may lack efficacy due to compensatory salvage pathways that can fill nucleotide pools when upstream steps are blocked. The flux models herein also indicate that GBMs adaptively rewire metabolism in response to radiation, perhaps explaining why these tumors typically recur following radiation treatment. Further, the dynamic ¹³C-MFA analysis revealed an increased reliance on de novo GMP synthesis after radiation, highlighting the important role that metabolic fluxes can play in treatment responses. Some of the flux models require sample acquisition at multiple timepoints and are not suitable for clinical application where tumor is typically removed only once. However, the serine production flux model herein requires only a single timepoint, so this model will have increased applicability in real world practice. The serine production model makes several simplifying assumptions (described in supplementary methods), as the ability to add complexity to the model depends on the underlying data. This study demonstrates the utility of simplified models in extracting quantitative information from metabolic studies. As experimental techniques grow, the data can be used to develop more complex models.

This work has several important clinical implications. Inhibiting nucleotide synthesis, serine uptake, or non-glucose TCA cycle fuel sources might have a therapeutic index to selectively affect GBM, whereas broadly targeting glucose uptake may cause unacceptably cortical toxicity. Targeting proximal de novo nucleotide synthesis in GBM may be ineffective due to active salvage pathways. Blocking IMPDH may still have therapeutic benefit in glioma with a favorable therapeutic ratio because of the preference for gliomas to salvage hypoxanthine. Restricting dietary serine could help slow GBM growth and potentially augment the efficacy of standard of care GBM treatments, though the efficacy of this approach could be limited by local production of serine in the GBM microenvironment. The patient-to-patient heterogeneity in environmental serine dependence observed suggests that isotope tracing could be used a precision medicine technique to determine which patients are mostly likely to benefit from dietary serine restriction.

This study is the first to directly measure biosynthetic flux in both glioma and cortical tissue in human brain cancer patients. Brain tumors rewire glucose carbon utilization away from oxidation and neurotransmitter production towards biosynthesis to fuel growth. Blocking these metabolic adaptations with dietary interventions slows brain cancer growth with minimal effects on cortical metabolism.

### Methods

### Clinical Stable Isotope Tracing Protocol

Eight patients with suspected GBM were recruited to the IRB-approved clinical study, which was performed perioperatively with standard-of-care craniotomies. Near the start of each procedure (approximately 2-4 hours prior to tissue resection), patients received a bolus intravenous dose of [U¹³C]-glucose (8 g) followed by a continuous intravenous infusion of [U¹³C]-glucose at a rate of 4 g/h. Arterial blood was collected into EDTA-coated vials every 30-60 min for plasma preparation and analysis until solid tissues of interest were harvested from each patient. At this point, radiographically defined enhancing tumor, non-enhancing tumor, and adjacent healthy cortical tissues were resected by the neurosurgeon (WNA), rinsed in cold PBS and immediately (<3 min after resection) flash-frozen in liquid nitrogen by the research team for further analysis.

### Animal Studies and Patient-Derived Xenograft Model

All animal studies were approved by the University Committee on Use and Care of Animals at the University of Michigan. In all animal experiments, male and female mice aged 4-12 weeks were used. Mice were housed in specific pathogen-free conditions at a temperature of 74° F. and relative humidity between 30 and 70% on a light/dark cycle of 12 h on/12 h off with unfettered access to food (PicoLab® Laboratory Rodent Diet, 5L0D) and water.

Studies assessing intracranial tumor-bearing mice used the PDX model GBM38, which is a chemoradiation resistant model that genetically and histologically represents a typical GBM, from the PDX National Resource at Mayo Clinic. Tumor tissue was propagated subcutaneously in the flanks of immunodeficient mice (B6.129S7-Rag1^{tm1Mom}/J [Rag1 KO], Jackson Laboratory). To introduce GFP and fluc into GBM tissue, flank tumors were used to generate short-term explant cultures and transduced by lentiviral infection (lenti-LEGO-Ig2-fluc-IRES-GFP-VSVG). Following infection, cells were enriched for GFP⁺ populations by fluorescence-activated cell sorting and then reintroduced to mice either as subcutaneous flank tumors or intracranial tumors. To generate orthotopic GBM brain tumors, 5×10⁵ GFP⁺ fluc⁺ GBM38 cells were stereotactically implanted into the region of the brain calculated to be the striatum in anesthetized Rag1 KO mice. Tumor development was then confirmed by bioluminescence imaging (BLI) as described below.

### Tumor Growth and Bioluminescence

To monitor intracranial tumor growth in mice, BLI was used, which leverages the expression of luciferase in intracranial tumors. For each measurement, mice intracranially implanted with fluc⁺ GBM38 cells were intraperitoneally injected with 150 mg/kg D-luciferin. Ten minutes after injection, mice were imaged using an IVIS™ Spectrum imaging system (PerkinElmer) while under anesthesia (2% isoflurane inhalation). In tumor growth studies, total fluxes of each tumor were normalized to time 0 flux, which is defined as the first day of detection after intracranial implant.

### Stable Isotope Infusions in GBM-Bearing Mice

At approximately 1-2 weeks before expected death related to brain tumors (˜3 weeks post-implant), intracranial GBM-bearing mice underwent dual catheterizations, with one catheter surgically placed into the jugular vein (for [U¹³C]-glucose administration) and a second catheter placed into the carotid artery (for plasma collection during infusion). Mice were then allowed to recover from surgery for 4-5 days. In studies assessing the influence of RT on metabolism, cannulated mice were anesthetized by 2% isoflurane inhalation and then treated with cranially directed RT at a dose of 8 Gy or sham RT with a lead shield keeping the cranium exposed. Immediately after RT (<5 min), awake and active mice were then administered a bolus dose of [U¹³C]-glucose (0.4 mg/g) followed by a continuous [U¹³C]-glucose infusion (12 ng/g/min) via the intravenous line for a total of 4 h. During infusions, blood was collected periodically via the carotid line into EDTA-coated vials and used to prepare plasma. At the end of infusions, ketamine (50 mg/kg) was administered into the intravenous line rapidly induce anesthesia. Mice were then decapitated, and tissues were extracted on dry ice. To separate orthotopic GBM from normal mouse cortex, we performed microdissection aided by a fluorescent bulb that allowed us to distinguish GFP⁺ tumor from GFP-cortex. All tissues were then immediately (<3 min post-anesthesia) flash-frozen in liquid nitrogen for further analysis.

### Liquid Chromatography-Mass Spectrometry

Flash-frozen tissue samples were homogenized in cold (−80° C.) 80% methanol. For plasma analysis, 100% methanol at −80° C. was added to plasma samples to yield a final methanol concentration of 80%. Insoluble material was then precipitated from all samples by centrifugation at 4° C., and supernatants containing soluble metabolites were dried by nitrogen purging. Dried metabolites were then reconstituted in 50% methanol for LC-MS analysis. Isotope labeling was determined using an Agilent system consisting of an Infinity Lab II UPLC coupled with a 6545 QTOF mass spectrometer (Agilent Technologies, Santa Clara, CA), and data were analyzed with values corrected for natural isotope abundance using MassHunter Profinder 10.0. We used control samples without ¹³C labeling to ensure that labeled isotopologs from [U¹³C]-glucose-infused mice and patients were not from contaminating species. To determine relative metabolite abundances in tissues and plasma from control or Ser/Gly (−) diet fed mice, samples were prepared as described above and then analyzed with an Agilent 1290 Infinity II LC-6470 Triple Quadrupole tandem mass spectrometer system (Agilent Technologies, Santa Clara, CA). For compound optimization, calibration, and data acquisition, Agilent MassHunter Quantitative Analysis Software version B.08.02 was used.

### MALDI Mass Spectrometry

Standard microscope slides with mounted tissue were vacuum desiccated for 20 minutes prior to matrix coating. After desiccation, slides were sprayed with NEDC matrix (10 mg/mL, 1:1 ACN:H₂O) using an M3+ sprayer (HTX Technologies LLC, Chapel Hill, North Carolina, flow rate: 75 μL/min, temperature: 70° C., velocity: 1000 mm/min, track spacing: 1 mm, pattern: crisscross, drying time: 0 sec). Slides were mounted into a MTP Slide Adapter II (Bruker Daltonics, Billerica, MA) before analysis.

MALDI imaging data were acquired using a timsTOF fleX MALDI-2 mass spectrometer (Bruker Daltonics) operating in transmission mode with a 20 μm raster size, acquiring m/z 100-600. The laser (Bruker Daltonics; SmartBeam 3D, 355 nm, 5000 Hz repetition rate) utilized a 16 μm beam scan, resulting in a 20 μm×20 μm ablation area. Taurine was used as a lock mass ([M-H]1−, m/z 124.0074).

Mass spectrometry imaging data were visualized using SCiLS Lab 2023b, with single fractional enrichment, normalized mean enrichment, and fractionalized enrichment images generated in SCiLS Lab using an in-house script utilizing the SCiLS REST API (Bruker Daltonics; version 6.2.114), written in R (version 4.2.2), using RStudio (2022.12.0 Build 353). A segmentation algorithm built into SCiLS Lab was used to create four data-driven regions corresponding to the healthy and GBM tissue in the ¹³C dosed and control tissues (normalization: total ion count, denoising: weak, method: bisecting k-means, metric: Manhattan). Relative isotopologue intensity of these regions was also calculated with another in-house script implemented through the SCiLS REST API.

Tentative annotations were performed using MetaboScape 2023 (Bruker Daltonics) using target lists of known biological molecules generated with the TASQ software (Bruker Daltonics; amino acids, glycolysis, citrate cycle, urea cycle, bile acid, gangliosides) as well as LipidBlast and LIPIDMAPS. The molecular formula of target molecules was used to calculate an accurate mass for each target. Annotations required a mass error of less than 3.0 ppm. In total, 72 features were annotated using this limited list, with annotated peaks having a mass accuracy of 1.1 ppm.

### Metabolic Flux Analysis

In vivo Metabolic Flux Analysis (IMFA) method was developed to estimate the purine and pyrimidine fluxes from isotopologue time-course data. A steady-state IMM-MFA method was used to estimate the serine contribution in human brain tumors.

iMFA at Metabolic Steady State

The model parameters comprised of reaction fluxes (expressed as vector v), the pool sizes of mass-balanced metabolites (expressed as vector c), the isotopologues of input metabolites (R), and the fraction of contribution of reactant isotoplogues to the product isotopologues (f) used only in pyrimidine model. The vector of model parameters, x is described in equation 8.

$$x = [ v , c , f , R ] \qquad Equation 8$$

Metabolites inside the model boundary were mass-balanced and are called balanced metabolites. The metabolites outside the model boundary were not mass-balanced and are called input metabolites. Due to the complicated time-dependent nature of in vivo metabolite enrichments, this demarcation between input metabolites and balanced metabolites was used to establish the model. A set of linear mass balance equations were used to describe overall mass balance. The sum of the isotopologues of the input metabolites was constrained to 1. Equation 9 describes the linear constraint equations. S is the stoichiometric matrix for a model with m balanced metabolites and n reactions. L is the linear constraints on the sum of input metabolite isotopologues, corresponding to p labeled input metabolites and r total isotopologues.

$$[ \begin{matrix}{S}_{m \times n} & {0}_{m \times m} & {0}_{m \times q} & {0}_{m \times r} \\ {0}_{p \times n} & {0}_{p \times m} & {0}_{p \times q} & {L}_{p \times r}\end{matrix} ] x = [ \begin{matrix}{0}_{m \times 1} \\ {1}_{p \times 1}\end{matrix} ] \qquad Equation 9$$

The time-dependent fractional isotopologue enrichment (MIDs) of balanced metabolites is described by a set of ordinary differential equations (ODEs, equation 10). The rate of change of the isotopologue d of metabolite i (M_{i,d}) is described by applying mass balance on the isotopologue.

$$\frac{{dM}_{i , d}}{dt} = \frac{1}{{c}_{i}} \cdot ( {\sum }_{{{S}_{ij > 0}}^{j = 1}}^{n} {S}_{i j} \cdot {v}_{j} ( {\prod }_{{{S}_{kj} < 0}^{{{\sum }_{} q = d}^{k}}} {M}_{k , q} ) + {\sum }_{{{S}_{ij} < 0}^{j = 1}}^{n} {S}_{i j} \cdot {v}_{j} \cdot {M}_{i , d} ) \qquad Equation 10$$

An objective function was minimized to solve the model and estimate the optimal parameters (equation 11). The objective function (obj) was calculated as the sum of square of the differences between the measured values of isotope enrichments (Mₑₓₚₜ) and the isotopic enrichments simulated by the model (Mₛᵢₘ) divided by the standard deviation of the experimental measurements (SDₑₓₚₜ). When the metabolite pool sizes were known, the difference between the known and simulated pool sizes were included in the objective function (cₑₓₚₜ−cₛᵢₘ).

$$obj = \sum {( \frac{{M}_{expt} - {M}_{s i m}}{S {D}_{expt}} )}^{2} + \sum {( \frac{{c}_{expt} - {c}_{sim}}{{SD}_{expt}} )}^{2} \qquad Equation 11$$

To estimate the fluxes and pool sizes, an initial parameter vector was selected randomly and the ODEs were solved to estimate the objective function. The objective function was minimized subject to linear constraints and parameter bounds. The optimization was performed in MATLAB with the Artlelys Knitro toolbox. The solver ode15s was used to solve ODEs as Initial Value Problems (IVPs). All metabolites were unlabeled at time t=0. The optimization was performed for 100 randomly generated initial parameter vectors and the chi-square-goodness-of-fit test was used to select the optimal parameter vector at 95% confidence. When the objective function was higher than the chi-square threshold, the parameter space with the lowest objective value was selected. The 95% confidence intervals for the estimated fluxes were determined.

Dynamic iMFA

The data for radiation-induced time-dependent change in metabolite pool sizes were incorporated into the model along with the MID-time profiles. The time-course flux profiles were parametrized by expressing them as B-splines (equation 12). B-spline is a parametric function that can be used to fit data without assuming a functional relationship between the input and output variables. It comprises of multiple polynomial segments joined together via ‘control points’. These control points control the shape of the b-spline curve and are hyperparameters in the model. Another hyperparameter is the b-spline order, which is equal to d+1, d being the degree of polynomials used to construct the b-spline.

$$v ( t ) = C P * N ( t ) \qquad Equation 12$$

The parameter vector comprised of the metabolite pool size at time t=0 (c₀), and the b-spline parameters expressed as vector cp and the isotopologues of input metabolites (R) (equation 13). The rate of change of metabolite pool size was expressed as a function of the stoichiometric matrix S and the time-dependent flux vector v(t) (equation 14).

$$x = [ cp , {c}_{0} , R ] \qquad Equation 13$$

$$\frac{dc}{dt} = S * v ( t ) \qquad Equation 14$$

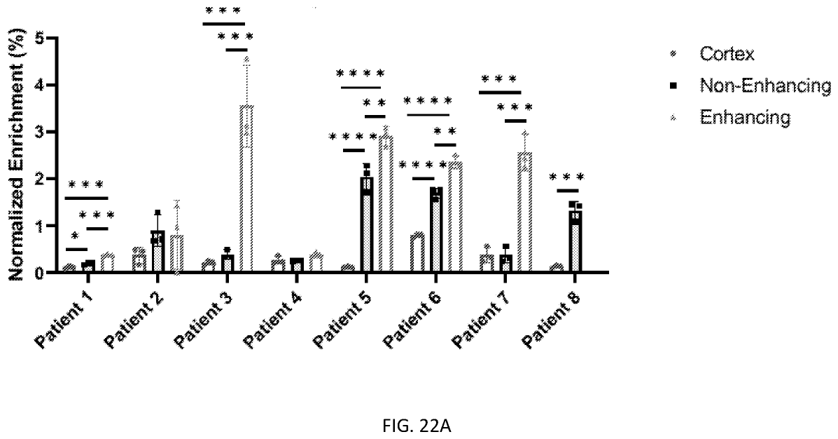

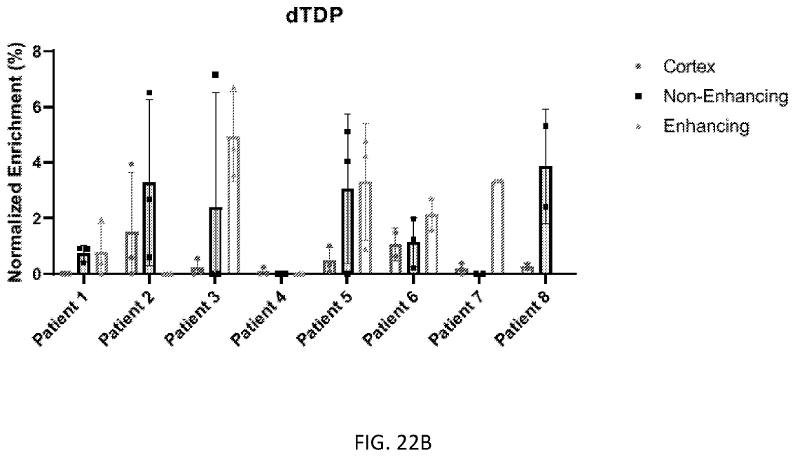

The objective function from the steady state iMFA model was modified to include the terms for time-dependent metabolite pool size (equation 15). Only the relative time-dependent pool size could be measured experimentally. Hence, the relative change of the metabolite pool size at time t(cₜ) relative to the pool size at time zero (c₀) was used in the objective function. The initial metabolite pool size values were the same as those used in the steady state model. The relative time-dependent concentration profiles are provided in FIG. 22B.

$$obj = \sum {( \frac{{M}_{expt} - {M}_{sim}}{{SD}_{expt}} )}^{2} + \sum {( \frac{{c}_{0 , expt} - {c}_{0 , s i m}}{{SD}_{expt}} )}^{2} + \sum {( \frac{{( \frac{{c}_{t}}{{c}_{0}} )}_{expt} - {( \frac{{c}_{t}}{{c}_{0}} )}_{sim}}{{SD}_{expt}} )}^{2} \qquad Equation 15$$

To estimate the flux profiles, an initial parameter vector was randomly selected, and the ODEs were solved to minimize the objective function as done for the steady state model. The flux values at t=0 were constrained to the 95% confidence intervals estimated from the steady state model. All metabolites were unlabeled at time t=0. The optimization was performed for 100 randomly generated initial parameter vectors and the parameter vector with the lowest objective value was selected. To estimate the 95% confidence intervals, the parameter bounds were estimated as done for the steady state model. The determined parameter bounds were used to create the time-course flux profiles corresponding to the 95% confidence interval. All the acceptable flux profiles were recorded and the minimum and maximum flux values at a certain time were reported as the 95% flux bound.

The b-spline hyperparameter selection was performed prior to parameter optimization. B-splines of orders 2, 3, and 4 were tested and a spline of order 3 (quadratic b-spline) was selected after qualitative analysis of the results. A randomized approach was used to select the b-spline control points. All flux profiles were assumed to have the same control points. Control points in the range of [0.2,0.8] were tested at intervals of 0.05. Each time, one position was selected, and parameter optimization was performed for the same initial parameter vector. The control point with the lowest objective was recorded. To optimize the placement of a second control point, the same strategy was used. The second control point was placed at a minimum distance of 0.2 from the first control point. The procedure was repeated for 100 initial guesses. For both the GBM and normal cortex samples, the addition of a second control point did not reduce the minimum objective value for the 100 optimizations. Hence, one control point was used to simulate both conditions. Based on the model fit, 0.3 was selected as the control point for GBM because of a lower mean objective value and central position. 0.65 was selected for the normal cortex.

### Metabolic Model of the Purine Pathway

The metabolic model of the purine synthesis pathway was curated on the basis of experimental data and current literature. The KEGG database was referenced for a list of reactions and the associated enzymes (Kanehisa, M. and Goto, S. (2000) KEGG: kyoto encyclopedia of genes and genomes. Nucleic Acids Res 28, 27-30). Data from the human proteome atlas was used to remove the reactions whose enzymes have low expression in GBM (Sjostedt, E. et al. (2020) An atlas of the protein-coding genes in the human, pig, and mouse brain. Science 367. All the reactions were assumed to be unidirectional and further simplifications were made based on the available experimental data. The net fluxes were assumed to be in the direction of purine synthesis. Further, IMP was assumed to directly produce GMP and the intermediate XMP was excluded from the model because the enrichment data for XMP was not available. All the ribose units (R5P, RIP, PRPP) were assumed to have the same enrichment. The final model comprised of 15 reactions and 23 metabolites (FIG. 25A, tables 4, 5). Six purine metabolites were inside the model boundary and were mass balanced. The rest of the metabolites are substrates in the purine pathway and outside the model boundary. The metabolites adenosine and hypoxanthine were assumed to be unlabeled based on experimental data. The enrichment data for guanine was not available and it was assumed to be produced mainly from the degradation of old DNA and RNA units and hence was unlabeled in the model. The cellular carbon dioxide pool was also assumed to be unlabeled. The enrichment of methyl units cannot be measured experimentally, and the values were estimated from the enrichments of serine and glycine.

TABLE 4
List of model reactions and associated flux bounds.

| | | | Flux Lower | Flux Upper |
|---|---|---|---|---|
| Reaction | | Reaction | Bound | Bound |
| No. | Reaction | Type | (pmol/mg · hr) | (pmol/mg · hr) |
| 1 | R5P + GLY + 2C—THF + CO₂ == IMP | Irreversible | 0.01 | 300 |
| 3 | IMP == INOSINE | Irreversible | 0.01 | 300 |
| 2 | R5P + HYPOXANTHINE == IMP | Irreversible | 0.01 | 300 |
| 4 | R5P + HYPOXANTHINE == INOSINE | Irreversible | 0.01 | 300 |
| 5 | INOSINE == 0 | Sink | 0.01 | 300 |
| 6 | IMP == GMP | Irreversible | 0.01 | 300 |
| 7 | R5P + GUANINE == GMP | Irreversible | 0.01 | 300 |
| 8 | GMP == GUANOSINE | Irreversible | 0.01 | 300 |
| 9 | R5P + GUANINE == GUANOSINE | Irreversible | 0.01 | 300 |
| 10 | GUANOSINE == 0 | Sink | 0.01 | 300 |
| 11 | GMP == GDP | Irreversible | 0.01 | 300 |
| 12 | GDP == 0 | Sink | 0.01 | 300 |
| 13 | IMP == AMP | Irreversible | 0.01 | 300 |
| 14 | ADENOSINE == AMP | Irreversible | 0.01 | 300 |
| 15 | AMP == 0 | Sink | 0.01 | 300 |

Reaction type irreversible refers to reactions that proceed only in one direction. Reaction type sink refer to the reactions that consume the metabolite and are not included in the model. Flux bounds were set according to the experimental data. AMP: adenosine monophosphate; C-THF: 5-methytetrahydrofolate; CO₂: carbon dioxide; GLY: glycine; IMP: inosine monophosphate; GDP: guanosine diphosphate; GMP: guanosine monophosphate; R5P: ribose-5-phosphate

TABLE 5
List of metabolites included in the model.

| Metab- | | | No. of |
|---|---|---|---|
| olite | | | Carbon |
| No. | Metabolite | Metabolite Type | Atoms |
| 1 | Ribose-5-Phosphate (R5P) | Input | 5 |
| 2 | Glycine (GLY) | Input | 2 |
| 3 | Carbon Dioxide (CO₂) | Input (unlabeled) | 1 |
| 4 | 5-Methyltetrahydrofolate | Input | 1 |
| | (C—THF) | | |
| 5 | Inosine Monophosphate (IMP) | Mass-Balanced | 10 |
| 6 | Inosine | Balanced | 10 |
| 7 | Hypoxanthine | Input (unlabeled) | 5 |
| 8 | Guanine | Input (unlabeled) | 5 |
| 9 | Guanosine Monophosphate (GMP) | Mass-Balanced | 10 |
| 10 | Guanosine Diphosphate (GDP) | Mass-Balanced | 10 |
| 11 | Guanosine | Mass-Balanced | 10 |
| 12 | Adenosine Monophosphate (AMP) | Mass-Balanced | 10 |
| 13 | Adenosine | Input (unlabeled) | 10 |

Input metabolites are not mass balanced. Some input metabolites had zero to low isotopic enrichment and were considered unlabeled.

To estimate the methyl unit enrichment, a pseudo-steady state assumption was applied. Serine, glycine, and the methyl enrichments were assumed to be in equilibrium at any given time. The corresponding equations for the metabolite isotopomers are represented in table 6 (SER: serine; GLY: glycine; ME: Me-THF; the number in subscript corresponds to the presence (1) or absence (0) of ¹³C carbon at the position). The variance-weighted sum-of-squared residuals was calculated between the experimental MID values of serine and glycine and the model estimated values. The optimization was performed from 10 initial guesses. To estimate the error in the estimated methyl enrichment, gaussian noise was added to the experimental values and the optimization was repeated 200 times.

TABLE 6
Equations to estimate methyl unit enrichment

| S. No. | Equation |
|---|---|
| 1 | SER₀₀₀ = GLY₀₀ME₀ |
| 2 | SER₀₀₁ = GLY₀₀ME₁ |
| 3 | SER₁₀₀₊₀₁₀ = GLY₁₀₊₀₁ME₀ |
| 4 | SER₁₀₁₊₀₁₁ = GLY₁₀₊₀₁ME₁ |
| 5 | SER₁₁₀ = GLY₁₁ME₀ |
| 6 | SER₁₁₁ = GLY₁₁ME₁ |
| 7 | ME₀ + ME₁ = 1 |

### Metabolic Model of the Pyrimidine Pathway

Pyrimidine model was implemented based on our available enrichment data for pyrimidines, the active reactions in KEGG database, and the enzymes that have high expression in GBM by checking the human protein atlas. The pyrimidine model consists of three reactions assumed to be unidirectional toward synthesis of UMP and listed in Table 7 and depicted in FIG. 25B. Flux bounds were selected based on our observation of optimization space and were relaxed to have no overlap with the flux confidence intervals.

TABLE 7
List of pyrimidine model reactions and associated flux bounds.

| | | | Flux | Flux |
|---|---|---|---|---|
| Reaction | | Reaction | Lower Bound | Upper Bound |
| No. | Reaction | Type | (pmol/mg · hr) | (pmol/mg · hr) |
| 1 | R5P + ASP + CO₂ == UMP + CO2 | Irreversible | 0.01 | 100 |
| 2 | URIDINE == UMP | Irreversible | 0.01 | 200 |
| 3 | UMP == 0 | Sink | 0.01 | 300 |

Reaction type irreversible refers to reactions that proceed only in one direction. Reaction type sink refer to the reactions that consume the metabolite and are not included in the model. Flux bounds were set according to the experimental data. UMP: uridine monophosphate; CO₂: carbon dioxide; R5P: ribose-5-phosphate; ASP: aspartate.

TABLE 8
List of metabolites included in the pyrimidine model.

| Metab- | | | No. of |
|---|---|---|---|
| olite | | | Carbon |
| No. | Metabolite | Metabolite Type | Atoms |
| 1 | Ribose-5-Phosphate (R5P) | Input | 5 |
| 2 | Aspartate (ASP) | Input | 4 |
| 3 | Carbon Dioxide (CO₂) | Input (unlabeled) | 1 |
| 4 | Uridine | Input | 9 |
| 5 | Uridine Monophosphate (UMP) | Mass-Balanced | 9 |

Input metabolites are not mass balanced. Some input metabolites had zero to low isotopic enrichment and were considered unlabeled.

The input metabolites of the model include R5P, aspartate, CO₂, and uridine (Table 8). We assumed that all ribose units including R5P, PRPP, and RIP share the same enrichment profiles. The only metabolite that is inside the model is UMP and we wrote the mass isotopologue balance on it (equation 16).

$$\frac{{dM}_{UMP , i}}{dt} = \frac{1}{{c}_{UMP}} ( {v}_{salvage} \cdot {M}_{URIDINE , i} + {v}_{de novo} \cdot {M}_{R 5 p , j} \cdot {\tilde{M}}_{ASP , k} - {v}_{{UMP}_{tot}} \cdot {M}_{UMP , i} ) ; j + k = i \qquad Equation 16$$

On the left side of the equation 16, the rate of change of UMP isotopologues is calculated. c_{UMP} denotes the concentration of UMP. The first term shows that UMP gets labeling from the salvage flux (v_{salvage}) through uridine MIDs (M_{URIDINE,i} where i denotes the M+i enrichment). The second term show UMP gets labeling from the de novo UMP flux (v_{de novo}) through R5P (M_{R5P,j}) and aspartate (\tilde{M}_{ASP,k}) MIDs. In this model, it was assumed that CO₂ is unlabeled. The produced UMP in de novo synthesis consists of nine carbons in which five carbons come from R5P to structure the ribose unit of UMP. The remaining four carbons in the uracil ring come from unlabeled CO₂ pool (2^{nd} position), and aspartate (4^{th}, 5^{th}, and 6^{th} positions). UMP de novo synthesis is a decarboxylation reaction and the carbon leaving the system as CO₂ might be labeled if the first carbon of aspartate is labeled. Therefore, we defined a new isotopologue distribution for aspartate (\tilde{M}_{ASP,k}) that only contributes to the UMP labeling. Table 9 shows aspartate isotopomers that correspond to each isotopologue of aspartate (M_{ASP,M+i}) that can enrich UMP in the same way. To do so, three isotopomer fractions were defined (f₁, f₂, f₃). For instance, suppose one labeled carbon in UMP comes from aspartate (\tilde{M}_{ASP,M+1}), since the labeling of the first carbon of aspartate does not affect the labeling of UMP, the following isotopomers of aspartate can produce one labeled carbon in UMP: 0001, 0010, 0100, 1001, 1010, and 1100. The defined fractions fᵢ corresponded to M+i isotopologue helped us to separate the isotopomers to isotopomers with labeled produced CO₂ and with unlabeled produced CO₂. For example, f₁ is the ratio of isotopomer 1000 to all isotopomers of M+1 (0001, 0010, 0100, 1000). To constrain the lower and upper bounds of fractions, we used the isotopomer distributions in mouse and human tumors reported in literature [10]. Hence, we set the constraints to [0.1, 0.4], [0.2, 0.6], and [0, 1] for f₁, f₂, and f₃, respectively. To combine the probability of production of UMP M+i isotopologues from aspartate and R5P, we multiplied M+j of R5P (M_{R5P,j}) to M+k of newly defined aspartate isotopologues (\tilde{M}_{ASP,k}) where j+k=i. The third term in equation 16 represents the consumption of UMP in other reactions outside of the boundaries of the model or the total UMP synthesis.

TABLE 9
Contribution of aspartate labeling in UMP labeling.

| Newly defined aspartate isotopologues | Aspartate isotopomers that | Aspartate isotopomers that |
|---|---|---|
| contributed to UMP labeling | produce unlabeled CO₂ | produce labeled CO₂ |
| \tilde{M}_{ASP, M+0} = M_{ASP, M+0} + f₁ M_{ASP, M+1} | 0000 | f₁: 1000 |
| \tilde{M}_{ASP, M+1} = (1 − f₁) M_{ASP, M+1} + | (1 − f₁): 0001, 0010, 0100 | f₂: 1001, 1010, 1100 |
| f₂ M_{ASP, M+2} | | |
| \tilde{M}_{ASP, M+2} = (1 − f₂) M_{ASP, M+2} + | (1 − f₂): 0011, 0101, 0110 | f₃: 1011, 1101, 1110 |
| f₃ M_{ASP, M+3} | | |
| \tilde{M}_{ASP, M+3} = (1 − f₃) M_{ASP, M+3} + | (1 − f₃): 0111 | 1111 |
| M_{ASP, M+4} | | |

### Metabolite Pool Sizes

Purine and pyrimidine pool sizes for mouse brain were obtained from literature, when available. Only the values reported with the measurement standard deviation were included in the objective function. When the measurement error was not available, the reported value was only used to set the bounds for the corresponding pool size parameter. Experimental data was used to estimate the pool sizes for GBM tissue. The average relative metabolite ion count between GBM and brain tissue was calculated and multiplied by the pool size in the brain. Standard error propagation techniques were used to estimate the standard deviation of the GBM pool sizes. GMP was not detected in the brain tissue but was detected in the GBM tissue. Hence, the bounds for GMP concentration were set to be higher than the pool size in the brain. The pool size data and source literature are reported in table 10.

TABLE 10
Purine and pyrimidine pool sizes used in metabolic model.

| | Pool Size in Mouse | Pool Size in GBM | Additional Bounds |
|---|---|---|---|
| Metab- | Brain (Mean ± SD, | (Mean ± SD, | on Pool Size |
| olite | pmol/mg tissue) | pmol/mg tissue) | (pmol/mg tissue) |
| GDP | 182.1 ± 8.2^{[11]} | 59.0 ± 22.1 | |
| Inosine | 126.7 ± 18.3^{[12]} | 94.1 ± 26.8 | |
| AMP | 172 ± 6.0^{[11]} | 95.4 ± 48.9 | |
| Guano- | 245.8 ± 31.6^{[12]} | 70.5 ± 22.0 | |
| sine | | | |
| GMP | 157 ± 40^{[13]} | | GBM: [172, 304] |
| IMP | ~200^{[14]} | | Brain: [125, 300] |
| | | | GBM: [30, 70] |
| UMP | 3.16 ± 1.26^{[15]} | 2.31 ± 1.10 | |

### Enrichment of Input Metabolites

The time-course isotopologue abundances of input metabolites are required to apply the metabolic model. The time-course enrichment of metabolites is complex in in vivo models and cannot be described by exact mathematical functions. A linear piecewise function was fit to the experimental time-point data to estimate the enrichment-time relationship of input metabolites. Equation 17 describes the calculation of the slope of the isotopologue R; between time points tₘ₊₁ and tₘ. A linear function was selected because it requires the least assumptions and does not overfit the data. To account for the uncertainty in the experimental measurements, the input isotopologue abundances were allowed to vary within one standard deviation of the experimentally measured mean value.

$$\frac{{dR}_{i} ( t )}{dt} = \{ \begin{matrix}\frac{{R}_{i} ( {t}_{2} ) - R ( {t}_{1} )}{{t}_{2} - {t}_{1}} & {t}_{1} \leq t < {t}_{2 \\} \\ … & \\ … & \\ \frac{{R}_{i} ( {t}_{m + 1} ) - {R}_{i} ( {t}_{m} )}{{t}_{m + 1} - {t}_{m}} & {t}_{m} \leq t < {t}_{m + 1}\end{matrix} \qquad Equation 17$$

### MFA of Serine Contribution in Human Tumors

The model comprised of three compartments which correspond to the cortex, enhancing tumor, and non-enhancing tumor (FIG. 6H). Each compartment had its own 3PG pool which was used for de novo serine synthesis within the compartment. The compartments were linked by a common input of external serine from circulation. A source of unlabeled serine was included in the model since serine might be derived from protein breakdown. Autophagy has been shown to be a source of serine in in vitro models of glioma [16]. Further, de novo serine synthesis is rate limited by the levels of the PHGDH enzyme, and serine may not be in isotopic equilibrium with 3PG. An unlabeled serine source helped correct for this effect. The net serine input to the model from de novo synthesis, external serine uptake, and the unlabeled serine from recycle was assumed to be 1 unit. Forward and reverse SHMT fluxes and the fluxes of glycine to 5,10-methylene-THF were included in each compartment. Serine was assumed to be the only source of glycine and one-carbon unit. This is in accordance with glycine being mainly derived from serine in the brain. Serine is also a major source of glycine and one-carbon units in gliomas. The enrichment of externally available serine was also assumed to be the same as the serine in circulation. Further, we assumed that tissues are homogenous and did not consider any cell-cell metabolic interactions in the serine-glycine pathway. These assumptions were important to reduce the model complexity and prevent the model from becoming highly underdetermined. Because of the various unknown factors, we only analyzed and reported the relative contributions of serine synthesis and uptake pathways that would yield labeled serine and did not analyze the absolute values. Sink fluxes were also included for serine, glycine, and formyl-THF to account for consumption not included in the model. The list of reactions and the associated carbon transitions are provided in table 11.

The model parameters x comprised of the fluxes v and the known IDV values D of 2PG/3PG, serine, and glycine in the three compartments. The MID of plasma serine was also included in the model parameters (equation 18). The fluxes in each compartment were mass balanced, which was described through the stoichiometric matrix S_{m×n} corresponding to m metabolites and n reactions (equation 19). The external serine was not included in the mass balance. To make sure that the sum of all IDVs of a certain metabolite is always equal to 1, the sums of the IDVs of the external serine and the three tissue 3PG were constrained to 1. This added 4 equations to the model (one for external serine, 3 for 3PG in the three tissue compartments). These equations were represented by the matrix L_{4×d} corresponding to d total IDV values in the model. The three equations constraining the new serine input to 1 were also included in the linear balance equations and are represented by the matrix N_{3×n}.

$$x = [ v , D ] \qquad Equation 18$$

$$[ \begin{matrix}{S}_{m \times n} & {0}_{m \times d} \\ {N}_{3 \times n} & {0}_{3 \times d} \\ {0}_{4 \times n} & {L}_{4 \times d}\end{matrix} ] x = [ \begin{matrix}{0}_{n \times 1} \\ {1}_{7 \times 1}\end{matrix} ] \qquad Equation 19$$

The IMM method was used to formulate the isotopic mass balance equations (equation 20) (Schmidt, K. et al. (1997) Modeling isotopomer distributions in biochemical networks using isotopomer mapping matrices. Biotechnol Bioeng 55, 831-840). These equations were used to add non-linear constraints to the model. Additional linear constraints were applied to avoid the model converging to a trivial solution. The parameters were optimized to minimize the objective function, which was defined to minimize the differences between the experimentally measured MIDs and the MIDs simulated by the model (equation 21). The difference between the experimental and simulated values was normalized to the experimental standard deviation to account for experimental variation. An L2 regularization term for the flux parameters was also included in the objective function because the model was underdetermined, i.e. we had more unknown parameters than the number of known values. The value of the regularization parameter was set to 0.1. To solve the model, local optimization was performed from 200 randomly selected initial points. Knitro toolbox was used for the optimization in MATLAB. The solution with the lowest objective value was selected. To estimate the 95% confidence intervals, gaussian noise was added to the experimental data and the optimization was repeated 1000 times. The distribution of the resulting solutions was used to determine the confidence intervals.

$${\sum }_{{{S}_{ij > 0}}^{j = 1}}^{n} {S}_{i j} \cdot {v}_{j} ( {\prod }_{{{S}_{kj} < 0}^{{{\sum }_{} q = d}^{k}}} {M}_{k , q} ) + {\sum }_{{{S}_{ij} < 0}^{j = 1}}^{n} {S}_{i j} \cdot {v}_{j} \cdot {M}_{i , d} = 0 ) \qquad Equation 20$$

$$obj = \sum {( \frac{\sum {y}_{s i m} - {M}_{expt}}{S {D}_{expt}} )}^{2} + 0.1 * {‖ [ v , y ] ‖}_{2} \qquad Equation 21$$

TABLE 11
List of reactions in the MFA model.

| S. No. | Reaction | Carbon Transition |
|---|---|---|
| 1 | SERₚₗₐₛₘₐ == SER_{cortex} | abc == abc |
| 2 | 3PG_{cortex} == SER_{cortex} | abc == abc |
| 3 | SER_{unlabeled} == SER_{cortex} | abc == abc |
| 4 | SER_{cortex} == GLY_{cortex} + Me—THF_{cortex} | abc == ab + c |
| 5 | GLY_{cortex} + Me—THF_{cortex} == SER_{cortex} | ab + c == abc |
| 6 | GLY_{cortex} == Me—THF_{cortex} + CO₂ | ab == b + a |
| 7 | SER_{cortex} == 0 | |
| 8 | GLY_{cortex} == 0 | |
| 9 | Me—THF_{cortex} == 0 | |
| 10 | SERₚₗₐₛₘₐ == SER_{enhancing} | abc == abc |
| 11 | 3PG_{enhancing} == SER_{enhancing} | abc == abc |
| 12 | SER_{unlabeled} == SER_{enhancing} | abc == abc |
| 13 | SER_{enhancing} == GLY_{enhancing} + Me—THF_{enhancing} | abc == ab + c |
| 14 | GLY_{enhancing} + Me—THF_{enhancing} == SER_{enhancing} | ab + c == abc |
| 15 | GLY_{enhancing} == Me—THF_{enhancing} + CO₂ | ab == b + a |
| 16 | SER_{enhancing} == 0 | |
| 17 | GLY_{enhancing} == 0 | |
| 18 | Me—THF_{enhancing} == 0 | |
| 19 | SERₚₗₐₛₘₐ == SER_{nonenhancing} | abc == abc |
| 20 | 3PG_{nonenhancing} == SER_{nonenhancing} | abc == abc |
| 21 | SER_{unlabeled} == SER_{nonenhancing} | abc == abc |
| 22 | SER_{nonenhancing} == GLY_{nonenhancing} + Me—THF_{nonenhancing} | abc == ab + c |
| 23 | GLY_{nonenhancing} + Me—THF_{nonenhancing} == SER_{nonenhancing} | ab + c == abc |
| 24 | GLY_{nonenhancing} == Me—THF_{nonenhancing} + CO₂ | ab == b + a |
| 25 | SER_{nonenhancing} == 0 | |
| 26 | GLY_{nonenhancing} == 0 | |
| 27 | Me—THF_{nonenhancing} == 0 | |

SER: serine;
GLY: glycine;
3PG: 3-phosphoglycerate;
Me—THF: 5,10-methylene-THF

TABLE 12
Estimated purine fluxes for GBM tissue. Flux bounds represent 95% confidence intervals.

| Reaction | | Flux | Lower Bound | Upper Bound |
|---|---|---|---|---|
| No. | Reaction | (pmol/mg · hr) | (pmol/mg · hr) | (pmol/mg · hr) |
| 1 | R5P + GLY + 2C—THF + CO₂ == IMP | 8.0 | 6.9 | 8.0 |
| 2 | IMP == INOSINE | 77.2 | 58.8 | 93.7 |
| 3 | R5P + HYPOXANTHINE == IMP | 250.4 | 214.7 | 263.1 |
| 4 | R5P + HYPOXANTHINE == INOSINE | 87.9 | 77.8 | 99.0 |
| 5 | INOSINE == 0 | 165.1 | 102.9 | 190.7 |
| 6 | IMP == GMP | 96.9 | 67.5 | 120.2 |
| 7 | R5P + GUANINE == GMP | 81.1 | 79.5 | 96.6 |
| 8 | GMP == GUANOSINE | 0.2 | 0.2 | 0.2 |
| 9 | R5P + GUANINE == GUANOSINE | 297.2 | 267.5 | 300.0 |
| 10 | GUANOSINE == 0 | 297.4 | 240.9 | 300.0 |
| 11 | GMP == GDP | 177.8 | 125.6 | 277.0 |
| 12 | GDP == 0 | 177.8 | 125.6 | 277.0 |
| 13 | IMP == AMP | 84.3 | 72.1 | 92.7 |
| 14 | ADENOSINE == AMP | 27.3 | 27.3 | 43.7 |
| 15 | AMP == 0 | 111.6 | 74.1 | 183.7 |

TABLE 13
Estimated purine fluxes for normal cortex tissue. Flux bounds represent 95% confidence intervals.

| Reaction | | Flux | Lower Bound | Upper Bound |
|---|---|---|---|---|
| No. | Reaction | (pmol/mg · hr) | (pmol/mg · hr) | (pmol/mg · hr) |
| 1 | R5P + GLY + 2C—THF + CO₂ == IMP | 1.0 | 0.1 | 3.4 |
| 2 | IMP == INOSINE | 1.6 | 0.1 | 61.5 |
| 3 | R5P + HYPOXANTHINE == IMP | 98.3 | 71.7 | 300.0 |
| 4 | R5P + HYPOXANTHINE == INOSINE | 24.3 | 13.5 | 71.0 |
| 5 | INOSINE == 0 | 25.9 | 17.6 | 46.5 |
| 6 | IMP == GMP | 7.9 | 0.1 | 65.3 |
| 7 | R5P + GUANINE == GMP | 93.6 | 75.8 | 300.0 |
| 8 | GMP == GUANOSINE | 17.1 | 1.9 | 41.4 |
| 9 | R5P + GUANINE == GUANOSINE | 45.4 | 20.4 | 81.5 |
| 10 | GUANOSINE == 0 | 62.6 | 37.1 | 109.0 |
| 11 | GMP == GDP | 84.3 | 63.6 | 300.0 |
| 12 | GDP == 0 | 84.3 | 63.6 | 300.0 |
| 13 | IMP == AMP | 89.8 | 71.5 | 285.4 |
| 14 | ADENOSINE == AMP | 0.0 | 0.0 | 16.2 |
| 15 | AMP == 0 | 89.8 | 76.3 | 300.0 |

TABLE 14
Estimated pyrimidine fluxes for GBM tissue. Flux bounds represent 95% confidence intervals.

| Reaction | | Flux | Lower Bound | Upper Bound |
|---|---|---|---|---|
| No. | Reaction | (pmol/mg · hr) | (pmol/mg · hr) | (pmol/mg · hr) |
| 1 | R5P + ASP + CO₂ == UMP + CO2 | 7.2 | 3.1 | 12.6 |
| 2 | URIDINE == UMP | 52.3 | 33.0 | 149.1 |
| 3 | UMP == 0 | 59.6 | 25.6 | 94.8 |

TABLE 15
Estimated pyrimidine fluxes for normal cortex tissue.
Flux bounds represent 95% confidence intervals.

| Reaction | | Flux | Lower Bound | Upper Bound |
|---|---|---|---|---|
| No. | Reaction | (pmol/mg · hr) | (pmol/mg · hr) | (pmol/mg · hr) |
| 1 | R5P + ASP + CO₂ == UMP + CO2 | 1.5 | 1.3 | 1.8 |
| 2 | URIDINE == UMP | 54.3 | 53.7 | 78.3 |
| 3 | UMP == 0 | 55.8 | 48.2 | 62.6 |

### Score of De Novo Serine Synthesis Relative to Cortex

A score was defined to compare the relative serine synthesis and uptake between the cortical and tumor tissues. The ratio of de novo serine synthesis to serine uptake was estimate for each tissue along with the 95% confidence interval of the ratio (FIG. 25B). The score was subsequently estimated by dividing the ratio of tumor tissues to that of matched cortical tissue.

### Dietary Restriction of Serine and Glycine

Three days prior to intracranial tumor implantation, mice were placed on either a control diet containing 1.00% serine and 0.99% glycine (TestDiet® Baker Amino Acid Diet, 5CC7), or a modified diet (TestDiet® Modified Baker Amino Acid Diet, 5BJX) containing 0% serine and 0% glycine with all other amino acids adjusted to account for serine and glycine reduction. Mice were then implanted with intracranial tumors and maintained on respective diets for the remainder of experimentation. Tumor growth was monitored by BLI. Once control mice neared humane endpoints, mice were deeply anesthetized, and blood was collected by cardiac puncture into EDTA-coated vials for plasma preparation. Mice were then decapitated, and brains were bisected through tumor tissue. One half of brain was fixed and embedded for histopathologic analysis. The other half of brain underwent rapid GFP⁺-based separation of tumor and cortex as described above. Tissues were then rapidly harvested on dry ice and flash frozen in liquid nitrogen for metabolomic analysis by LC-MS on an Agilent 6470 mass spectrometer as described above.

### Statistical Analysis of Metabolite Enrichment

Statistical analysis was performed either in R or in GraphPad Prism. Data were tested for normal distribution using the D'Agostino normality test (n>=8) or the Shapiro-Wilk normality test (n<7). For normally distributed data, a t-test was used for two groups, and a one-way ANOVA followed by Tukey HSD was used for more than two groups. For non-normal data, a Mann-Whitney U test was used for two groups. For multiple groups, the Kruskal-Wallis test was used, followed by Mann-Whitney U test with Bonferroni correction for pairwise comparison. To reduce chances of false positives in the differential enrichment analysis, FDR correction was used.

### Example 4

The most prevalent and aggressive brain tumor, glioblastoma (GBM), is distinguished by severe invasiveness and treatment resistance. The standard course of treatment for GBM entails surgical resection, followed by radiation therapy (RT) and temozolomide chemotherapy. GBMs typically recur despite current treatments, and the majority of patients die within 1-2 years after diagnosis.

Cancer cells have metabolic changes that provide them access to both conventional and unconventional nutrient sources. They utilize these nutrients to produce new biomass to support their proliferation. This metabolic rewiring represents a targetable liability with a therapeutic window.

One way to study cancer metabolism is isotope tracing. It is possible to determine which metabolic pathways are active in a system by following these isotopes to their downstream metabolites using mass spectrometry.

U13C-glucose was administered to GBM patients and GBM patient-derived xenografts (PDXs) and higher synthesis of purines and pyrimidines was observed in glioma samples which is in agreement with their need for nucleotides to sustain their regulated proliferation. Nucleotide synthesis consists of a salvage pathway wherein free bases react with phosphoribosyl pyrophosphate (PRPP), and de novo synthesis in which carbons and nitrogens from numerous sources are combined with PRPP. Understanding the level of contribution of these pathways in the synthesis of nucleotides is beneficial in that if de novo synthesis is the primary source of nucleotide synthesis, medications targeting de novo reactions such as mycophenolate mofetil (MMF) or 5-fluorouracil can be effectively employed. However, because all these pathways generate labeled metabolites, enrichment data alone cannot be utilized to determine whether the purines are being generated via de novo pathways or salvage pathways. Hence, quantification of salvage and de novo synthesis fluxes is beneficial.

The quantification of metabolic fluxes can be performed using metabolic flux analysis (MFA) which uses enrichment data at steady state, or isotopic non-steady state MFA (INST-MFA) which uses time course enrichment data. Herein, U13C-glucose was infused in patients at the time of craniotomy which usually takes between 2-4 hrs. The patient enrichment data of purines cannot be used in conventional MFA methods to quantify salvage and de novo synthesis because the enrichments are at a single time point, and purine metabolism is slow, and the enrichment of purines cannot reach steady state during the time of surgery.

Enrichment data show that among all nucleotides, guanosine monophosphate (GMP) was the only metabolite that had significantly higher enrichment in all glioma samples compared to normal cortex in both patients and PDXs. This suggests the synthesis of GMP is essential for gliomas to proliferate. Determining the level of contribution of salvage and de novo pathways in GMP synthesis is of interest. The drug MMF inhibits the enzyme IMPDH (inosine monophosphate dehydrogenase), which catalyzes de novo GMP synthesis. Accordingly, if GMP is majorly produced through de novo synthesis in a patient, MMF treatment might be administered for that patient. Otherwise, MMF might not be beneficial for that patient.

To estimate the contribution of de novo GMP synthesis to the overall GMP synthesis, machine learning models were implemented. There are two main advantages of machine-learning based models to estimate contribution of fluxes: (1) Machine learning can identify complex patterns that may be missed by traditional approaches. Hence, machine learning may be able to identify a structure in the enrichment data to estimate pathway contributions from single time point enrichment patterns. (2) Machine learning models are easier and faster to implement compared to mathematical MFA models that require more time and expertise to implement. (3) Once a machine learning model is trained, the response of prediction is quick, and it can be used by clinicians to suggest personalized treatments. Hence, a machine learning framework was established herein to indicate which patients might benefit most from targeting GMP de novo pathway by quantifying the contribution of de novo GMP synthesis to the overall GMP synthesis.

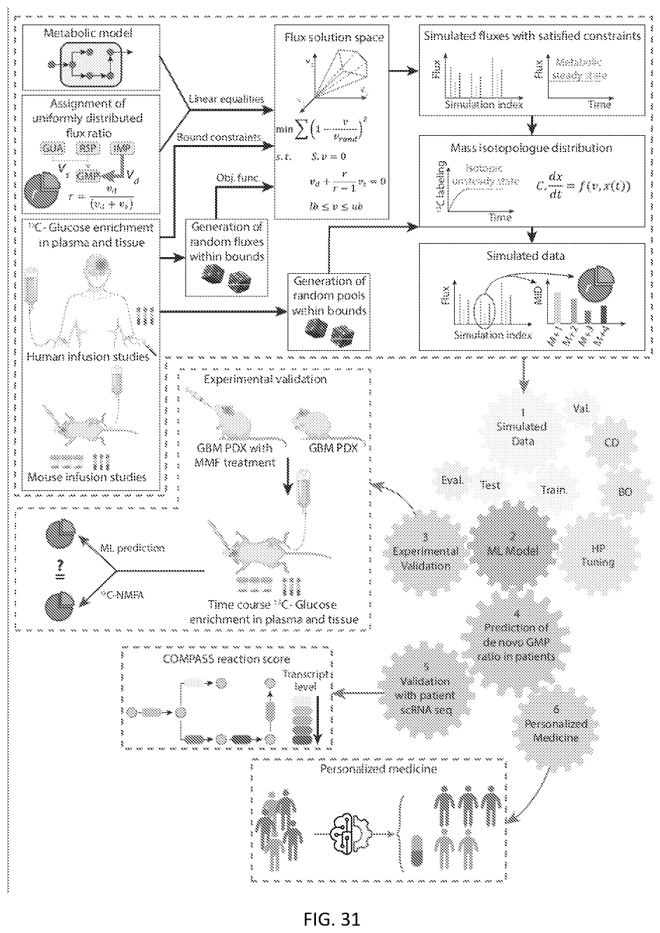

An overview of methods is shown in FIG. 31. To train machine learning models that can predict the ratio of de novo synthesis of GMP to the overall GMP synthesis, simulated MIDs were used as input features. The data was split into training, validation, and test datasets. Training and validation datasets were used to train the data and evaluate the model performance in each epoch. In addition, validation dataset was used to tune hyperparameters of the model. The criteria we used in hyperparameter tuning was the coefficient of determination (CD) between the actual and predicted values of the validation dataset using Bayesian optimization (BO). The test dataset was used to provide an unbiased evaluation of a final model fit on the unseen data. A machine learning model with a good evaluation score on a test dataset should be also evaluated on some experimental value for further validation. The designed experiment consisted of two conditions: GBM PDXs control cohort and GBM PDX treated with MMF. PDXs were infused with U13C-glucose and plasma and tissue samples were collected at multiple time points. By measuring the MIDs in these two groups, the de novo GMP ratio can be calculated using the INST-MFA method and compared to machine learning results. The INST-MFA method, intuitively, should result in lower de novo GMP ratio in the MMF treated cohort. After experimental validation, machine learning models can be applied to patients. Both patient MIDs and scRNA-seq data was available for the experiments herein. The de novo GMP ratio can be calculated via flux balance analysis and compared to our machine learning results. This method can be applied in a clinical trial in which machine learning predicts which patients may benefit from MMF treatment.

### Simulation of Mass Isotopologue and Flux Distribution to Train Machine Learning

Simulation of mass isotopologue and flux distributions is used herein, in part because supervised machine learning needs features and their corresponding targets of numerous samples; and in part because patient samples are limited and the fluxes (i.e., targets) are unknown. The framework of data simulation is shown in FIG. 31. To simulate purine MIDs and fluxes, a metabolic model that includes the upstream of metabolites producing purines de novo was used. The metabolic model enforces the fluxes to follow stoichiometric mass balances. It was assumed that fluxes and pools are at steady state while MIDs are not. The ratio of de novo GMP synthesis to the overall GMP synthesis (i.e., target of machine learning model) was set to a uniform distribution within the range of [0, 1] which helps the machine learning model to learn this prediction with the same frequency. A non-linear optimization problem was defined to simulate mass-balanced random fluxes (Eq. 22). To solve this initial value problem, a set of uniformly distributed random fluxes and pools was generated in each iteration. Previous INST-MFA of time-course metabolite enrichments in GBM PDXs, were able to determine the fluxes of purine metabolism. Utilizing MFA, enrichment of time-course patient plasma samples and a multicompartment model of cortex, non-enhancing, and enhancing tumors, serine de novo and salvage fluxes were determined. To limit the distribution of initial values of fluxes and constrained fluxes in our optimization, the flux bounds were set based on INST-MFA and MFA fluxes.

ScRNA-seq of patients and flux balance analysis of patients can be used for further validation of flux bounds. The fmincon function in MATLAB was used to solve the optimization problem. By solving the optimization problem, a set of constrained fluxes for each iteration was calculated.

$$min \sum {( 1 - \frac{v}{{v}_{r a n d}} )}^{2} \qquad Eq . ( 22 )$$

$$s . t .$$

$$S . v = 0$$

$${v}_{d} + \frac{r}{r - 1} {v}_{s} = 0$$

$$lb \leq v \leq u b$$

Then, by writing the mass balance on isotopologues and isotopomers (Eqs. 23 and 24) and solving a system of ordinary differential equations (ODEs), time-course MIDs were simulated for each set of fluxes. This process was repeated for 50,000 times to generate enough samples for machine learning training.

$$\frac{{dM}_{i , d}}{dt} = \frac{1}{{c}_{i}} \cdot ( {\sum }_{{{S}_{ij} > 0}^{j = 1}}^{n} {S}_{i j} \cdot {v}_{j} ( {\prod }_{{{S}_{kj} < 0}^{{\sum q = d}^{k}}} {M}_{k , q} ) + {\sum }_{{{S}_{ij} < 0}^{j = 1}}^{n} {S}_{i j} \cdot {v}_{j} \cdot {M}_{i , d} ) \qquad Eq . ( 23 )$$

Where in Eq. 23, the rate of change of isotopologue d of metabolite i (M_{i,d}) is calculated from the isotopologues of other metabolites that produce M_{i,d} (first term), and the isotopologues that consume M_{i,d} (second term). Note that Eq. 23 can be used if a metabolic model contains only concentration reactions i.e., there is only one product per reaction. For cleavage reactions, contribution of isotopomer patterns in mass balance equations is used (Eq. 24).

$$\frac{{dM}_{i}}{dt} = \frac{1}{{c}_{i}} \cdot ( {\sum }_{{{S}_{ij} > 0}^{j = 1}}^{n} {S}_{i j} \cdot {v}_{j} ( {\prod }_{{{S}_{kj} < 0}^{k}} {M}_{k} \cdot {IMM}_{k \to i} ) + {\sum }_{{{S}_{ij} < 0}^{j = 1}}^{n} {S}_{i j} \cdot {v}_{j} \cdot {M}_{i} ) \qquad Eq . ( 24 )$$

Here, the rate of change of the isotopomer vector i (Mᵢ) is expressed as a function of the reaction fluxes v, stoichiometric matrix S, and the concentration of the metabolite ci. The reactions that produce or consume metabolite i are represented by j. Index k refers to the metabolites that are converted to i. Mₖ is the isotopomer vector of k and the atom transition from k to i is denoted by the matrix IMM_{k→i}.

### Metabolic Model

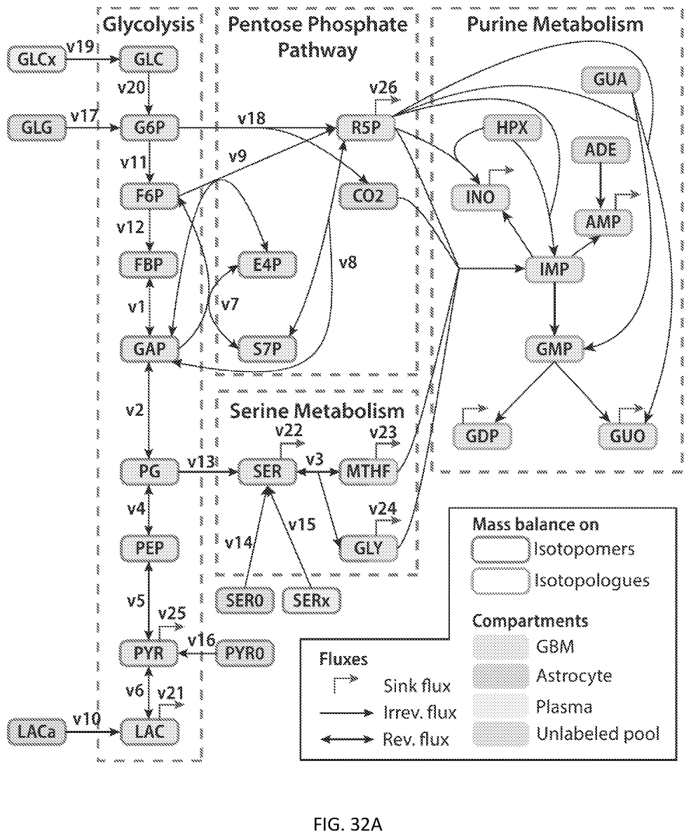

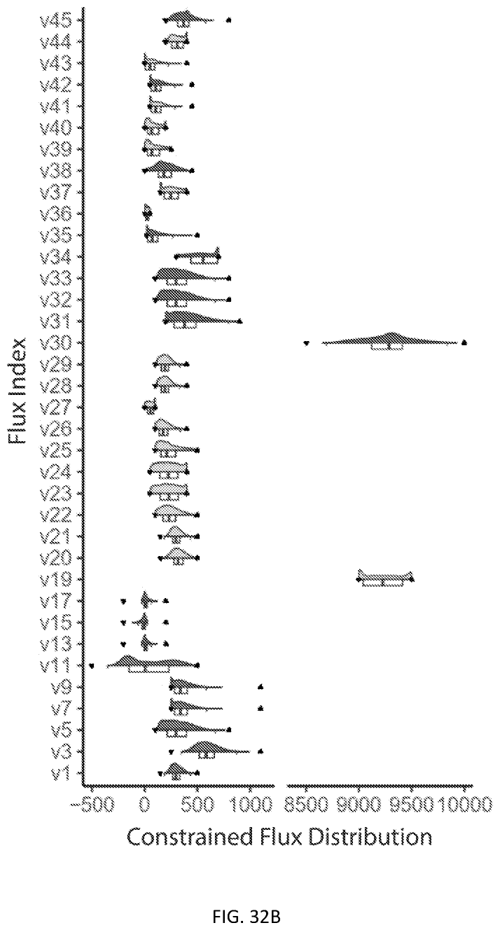

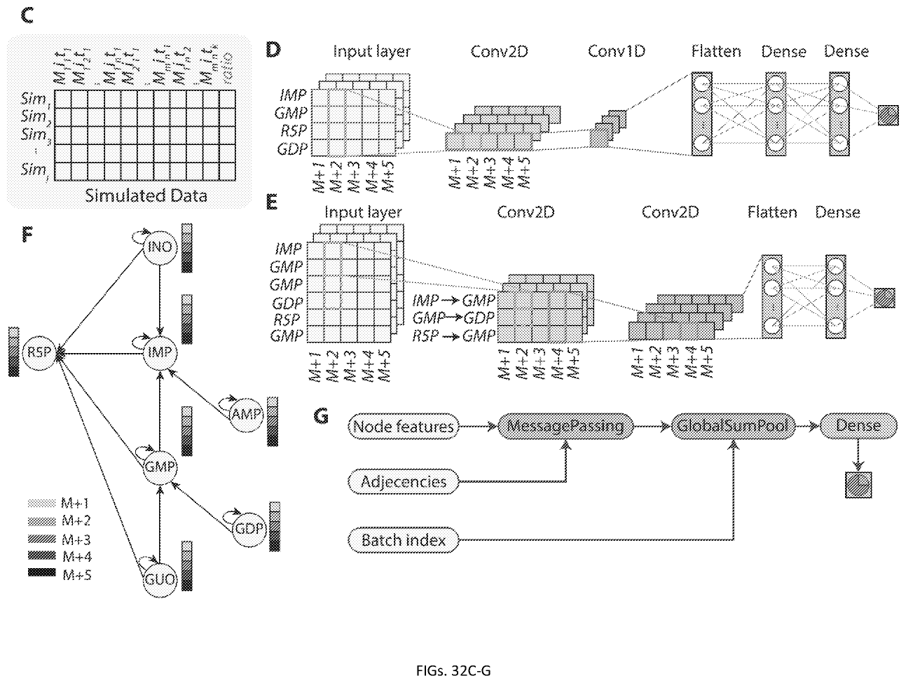

A metabolic map consisting of glycolysis, pentose phosphate pathway (PPP), serine metabolism, and purine metabolism was employed in flux simulation (FIG. 32A). Some metabolites were added to the metabolic model to encompass the patient MIDs and different compartments. They include enrichments of plasma glucose and serine as input metabolites (GLCx and SERx). The plasma glucose was assumed to have enrichment patterns of M+0 or M+6. The enrichment of M+6 glucose was estimated with Eq. 25 [1].

$$G L {C}_{M + 6} = G L {C}_{M + 6 , st . st .} + ( \frac{P}{P + Q} - G L {C}_{M + 6 , st . st .} ) {e}^{- \frac{r ( 1 - {GLC}_{M + 6 , st . st} ) t}{Q + G L {C}_{M + 6 , st . st .}}} \qquad Eq . ( 25 )$$

Where, GLC_{M+6,st.st.} is the steady state value of M+6 glucose in the plasma and was selected in the range of [0.2, 0.45] based on our patient plasma enrichments. Q is the total amount of glucose in the body and was randomly set to a value between 3 g and 7 g, which is the physiological range for humans (fasting glucose concentration is 72-108 mg/dL, the average human has 5.5 L blood) [2-4]. r and P are the rate of glucose infusion and the bolus dose, respectively. In our experiments, r is 4 g/hr and P is 8 g. Plasma serine was assumed to have M+0 and M+1 labeling based on our patient plasma enrichments. M+1 serine was assumed to be present in the 001 isotopomer form. Serine M+1 was assumed to be at steady state and assigned a randomly selected value between 0.2 and 0.65 based on our patient plasma enrichments. Additionally, astrocytic lactate (LACa) was another metabolite added to the metabolic model. Astrocytes are key regulators of central carbon metabolism and known to have a high conversion of glucose to lactate. Astrocytes secrete lactate into the extracellular environment where it can be used by other cell types. The described metabolic model was used to constrain random fluxes (Eq. 22). The stoichiometric constraints defined by this metabolic model changed the distribution of random fluxes from uniform to unique distributions governed by mass conservation rules (FIG. 32B). A system of ODEs consisting of isotopomer mass balances (Eq. 24) was solved to estimated time-course isotopomers of metabolites in glycolysis, serine metabolism and PPP as shown in FIG. 32A. The input metabolites of purine metabolism from other pathways are ribose 5-phosphate (R5P), CO₂, 10-formyl tetrahydrofolate (MTHF), and glycine (GLY). The MIDs of these metabolites was calculated by summing over isotopomers that contributes to an isotopologue. Then, another system of ODEs was solved which describes isotopologue mass balances on purine metabolites (Eq. 23).

### Data Processing for Machine Learning Models

Although the simulated data includes many metabolites from glycolysis, serine metabolism, and PPP, only purine metabolites (IMP, GMP, guanosine diphosphate (GDP)) and R5P were kept to configure input data with M+1 to M+5 labeling. Additionally, since these metabolites produce/consume GMP, they affect the de novo GMP ratio directly. The shape of simulated data is shown in FIG. 32C, where each row represents the MIDs (iₙ) of different metabolites (Mₘ) at multiple time points (tₖ) in one simulation with one set of fluxes (Simⱼ); where n is the no. of MIDs, m is the no. of metabolites, k is the no. of time points, and j is the no. of simulations. Few simulations included negative MIDs and infeasible solutions of the flux optimization problem and hence were removed. Data was split into 85% training and 15% test datasets by random sampling across simulations. Data was standardized according to mean and standard deviation of training data. This way, we reduced the effect of time variance of MIDs on our steady state target. The training data was further split into 85% training and 15% validation datasets. The data was split into features and target (ratio of de novo GMP synthesis).

### Convolutional Neural Network (CNN) Design

Input features were reshaped to a 4-dimensional (4-D) matrix (no. of samples in each data set*k, m, n, 1). The input layer of CNN shown in FIG. 32D is configured based on the shape of the input data to be (m, n, 1). The input layer is followed by a 2-D convolution layer (Conv2D) with a kernel size of (m, 1) which results in (1, n) output. The Conv1D layer applied on this output has a kernel size of (n,) which results in a single value. A flatten layer flattens these values across all kernels and enters a fully connected network with two dense layers. The intuition behind this network is that the Conv2D captures the dependencies between different metabolites with the same MIDs and the Conv1D captures the total labeling.

Another CNN was also implemented based on the reactions that include purine metabolites and R5P (FIG. 32E). Input features were reshaped to a 4-D matrix (no. of samples in each data set*k, no. of reactions that include Mₘ*2, n, 1). The intuition behind this model is to capture a relationship between labeling of reactants and products. Since there are three reactions that affect the GMP labeling directly and there is one reactant and one product labeled in these reactions, we configured our first layer with a shape of (no. of reactions*2, n, 1). A Conv2D layer with the kernel size of (2, 1) and stride of (2, 1) applies on the input layer which results in the (no. of reactions, n, 1) output. This output describes reactions that produce/consume GMP which is followed by another Conv2D layer that captures the relationship between GMP production reactions and GMP consumption reactions. The output of this layer enters a Conv1D layer to capture the total labeling. Then, a flatten layer provides a suitable form of Conv1D output to enter a dense layer. One structural advantage of CNN model shown in FIG. 32E than the CNN model shown in FIG. 32D is that it has fewer trainable parameters since it has more convolution layers and fewer dense layers.

To train the CNN, the trainable parameters such as the weights of layers must be optimized such that a loss function is minimized. A convolution layer consists of a kernel with trainable weights that cross-correlate to its previous layer output. A mini batch gradient descent approach with the size of 256 was selected to update all the weights according to the gradient of loss function using Adam optimizer with learning rate of 0.004 and epsilon of 0.4. Since the de novo GMP ratio is a continuous variable, a regression model is required to predict it. Hence, the loss function was set to the mean squared error (MSE). For all layers, no bias term, ReLU activation function, L2 kernel regularizer, HeUniform kernel initializer were considered. A batch normalization layer applied after each activation function. These functions were utilized from the TensorFlow Keras library in python.

### Graph Neural Network (GNN) Design

Another machine learning model that is suitable for the reaction network is graph neural network (GNN). The data processing of GNN is similar to CNN but with three differences: (1) simulated MIDs of 7 metabolites including R5P, inosine (INO), IMP, GMP, GDP, adenosine monophosphate (AMP), and guanosine (GUO) were kept; (2) features were reshaped to a 3-D matrix (no. of samples in each data set*k, m, n); (3) input data has to be in graph format with defined nodes and edges. To customize the 3-D matrix of features into a directed graph, a network of metabolites was established as nodes. A directed edge from node B to node A if reaction A→B exists was added. NetworkX library in python was used to build the graph in FIG. 32F and determine the graph adjacency matrix. The intuition behind this graph structure is based on mass isotopologue balances. For example, the isotopologue mass balance on GMP is shown in Eq. 26 where the rate of change of GMP M+i is shown on the left. On the right, the first two terms show production of GMP M+i, while the third term shows the consumption of GMP M+i. To analogize Eq. 26 to a graph, we added directed edges from GMP to IMP and R5P and a self-loop on GMP for the GMP consumption term.

$$\frac{{dM}_{G M P , i}}{dt} = \frac{1}{{c}_{G M P}} ( {{v}_{IMP}}_{\to GMP} \cdot {M}_{IMP , i} + {v}_{GUA + R5P \to GMP} \cdot {M}_{R 5 P , i} - \text{} ( {v}_{GMP \to GDP} + {v}_{GMP \to GUO} ) \cdot {M}_{G M P , i} ) \qquad Eq . ( 26 )$$

To create training, validation, and test graphs, Spektral library in python was used where the 3-D matrix of features was reconstructed to no. of samples*k graphs with m nodes connected according to the predefined adjacency matrix and each node has n features (M+1 to M+5). A minibatch gradient descent was administered to update node embeddings based on Eq. 27 where H^{(l)} is the updated node embeddings, A is the adjacency matrix with the shape of (m, m), H^{(l-1)} is the previous node embeddings with the shape of (m, n), W^{(l)} represents the trainable weights with the shape of (n, n), and σ is an activation function.

$${H}^{( l )} = σ ( {AH}^{( l - 1 )} {W}^{( l )} ) \qquad Eq . ( 27 )$$

The advantage of GNN over CNN is that the kernel in CNN has a constant shape, but in GNN, node connectivity can be defined by adjacency matrix. A metabolite might have a degree of one, while another metabolite might have a degree of ten; hence a CNN with constant shape of kernel cannot cross-correlate to the MIDs of a broader metabolic network and resembling the metabolic connectivity is only possible through GNNs.

Since there at most 2-hop connectivity in the proposed graph, the GNN can benefit from 1 or two message passing layers (FIG. 32G). Prediction of de novo GMP synthesis is a graph-level regression task, thus a pooling layer such as GlobalSumPool was used to flatten the graph convolution output which can be entered a fully connected layer to predict the ratio of de novo GMP synthesis to the overall synthesis.

### Hyperparameter Tuning

Bayesian optimization was used to tune hyperparameters of machine learning models using Optuna library in python. These hyperparameters include number of neurons in dense layers, number of dense layers, number of filters in convolution layers, Adam optimizer hyperparameters such as learning rate and epsilon. In each trial of combination of different hyperparameters, the model is trained and evaluated on validation dataset. Bayesian optimization has a surrogate function that helps it to converge faster to the optimal set of hyperparameters by skipping some hyperparameter combinations. The Bayesian model tries to maximize the coefficient of determination between predicted and actual labels of validation dataset.

### Results

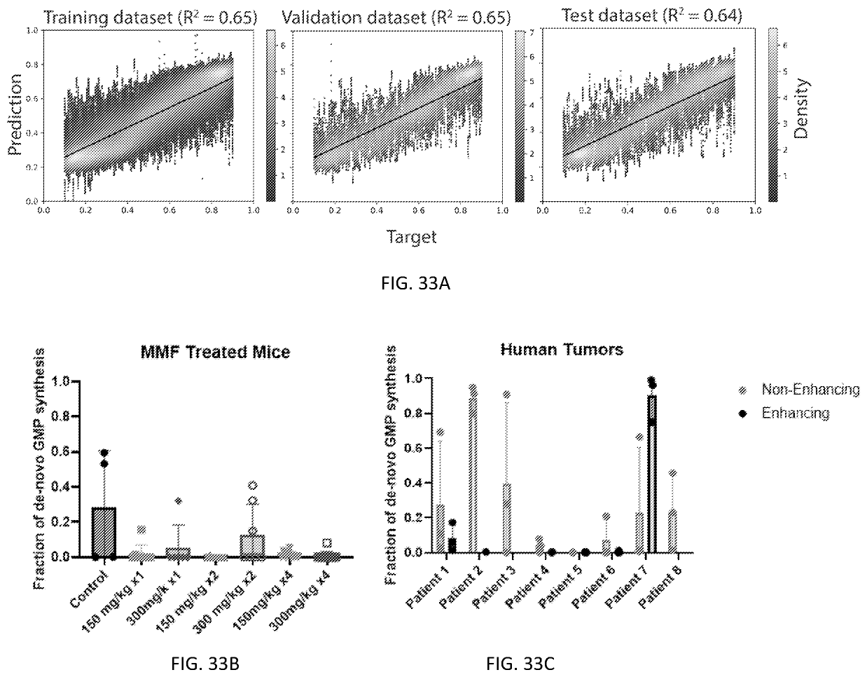

The model was evaluated on the training and test datasets, and a similar correct prediction rate was seen which suggests the model doesn't overfit and is generalizable on unseen data (FIG. 33A). To validate the machine learning model, GBMs were grown in PDXs and treated some with MMF and infused U13C glucose and measured metabolite enrichment patterns in both conditions. Since MMF targets IMPDH enzyme or de novo GMP synthesis, lower de novo GMP synthesis ratio in MMF treated mice would be expected. Consistent with this, the machine learning model predicted lower de novo GMP synthesis ratio in MMF treated mice (FIG. 33B). The GMP de novo synthesis ratio in patient tumors was then predicted (FIG. 33C). Enhancing and non-enhancing tumor samples were classified based on their histology in contrast imaging. Non-enhancing tumors have lower penetration of blood vessels than enhancing tumors. The model predicted almost consistent results among patients comparing enhancing and non-enhancing tumors with non-enhancing tumors having higher de novo GMP synthesis.

The ratio of de novo GMP synthesis can be quantified using the time course U13C glucose tracing and INST-MFA model the INST-MFA results can be compared with the machine learning results. Fluxes can be estimated using the sc-RNA seq data from patients and flux balance analysis and these estimations can be compared with the machine learning predictions.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

We claim:

1. A method of estimating absolute metabolic flux for a metabolic pathway of interest, the method comprising:
a) administering a tracer to subject;
b) collecting a sample from the subject; and
c) generating an estimate of absolute metabolic flux for a pathway of interest.

2. The method of claim 1, wherein the estimate of absolute metabolic flux for the pathway of interest is generated based upon a level of one or more metabolites and/or isotopologues thereof in the sample at a single point in time following administration of the tracer to the subject.

3. The method of claim 1 or claim 2, wherein the pathway of interest is a purine synthesis pathway.

4. The method of claim 3, wherein the one or more metabolites and/or isotopologues thereof comprise one or more of Ribose-5-Phosphate (R5P), Glycine (GLY), Carbon Dioxide (CO2), 5-Methyltetrahydrofolate (C-THF), Inosine Monophosphate (IMP), Inosine, Hypoxanthine, Guanine, Guanosine Monophosphate (GMP), Guanosine Diphosphate (GDP), Guanosine, Adenosine Monophosphate (AMP), and Adenosine.

5. The method of claim 3 or claim 4, further comprising determining a contributing fraction of de novo GMP synthesis and/or de novo IMP synthesis in the purine synthesis pathway.

6. The method of claim 1 or claim 2, wherein the pathway of interest is a pyrimidine synthesis pathway.

7. The method of claim 6, wherein the one or more metabolites and/or isotopologues thereof comprise one or more of Ribose-5-Phosphate (R5P), Aspartate (ASP), Carbon Dioxide (CO2), Uridine, and Uridine Monophosphate (UMP).

8. The method of claim 1 or claim 2, wherein the pathway of interest is a serine synthesis pathway.

9. The method of claim 8, wherein the one or more metabolites and/or isotopologues thereof comprise one or more of 3-phosphoglycerate (3PG), glycine (gly), and 5,10-methylene-THF (Me-THF).

10. The method of any one of claims 1-9, wherein the sample is a tissue sample.

11. The method of claim 10, wherein the subject has cancer, and wherein the tissue sample comprises a tumor tissue sample.

12. The method of claim 1, wherein the cancer is a brain cancer.

13. The method of claim 12, wherein the brain cancer is a glioblastoma.

14. The method of any one of claims 11-13, wherein the estimate of absolute metabolic flux for the pathway of interest is generated based upon a level of one or more metabolites and/or isotopologues thereof in the sample at a single point in time following administration of radiation therapy to the subject.

15. The method of any one of claims 1-14, wherein the estimate of absolute metabolic flux for the pathway of interest is generated using an artificial intelligence/machine learning (AI/ML) model.

16. The method of any one of claims 1-15, wherein the tracer comprises a moiety labeled with ¹³C, ¹⁵N, ¹⁸O, or ²D.

17. The method of claim 16, wherein the moiety comprises glucose, methionine, serine, glutamine, lactate, acetate, hypoxanthine, or uridine.

18. A method of selecting a therapy for a subject in need thereof, the method comprising:
a) generating an estimate of absolute metabolic flux for a pathway of interest using the method of any one of claims 1-17, and
b) selecting a therapy for a subject in need thereof based upon the estimate of absolute metabolic flux for the pathway of interest.

19. The method of claim 18, wherein the subject has cancer, and wherein the pathway of interest is a cancer-related pathway.

20. The method of claim 19, wherein the cancer is a brain cancer.

21. The method of claim 20, wherein the brain cancer is a glioblastoma.

22. The method of any one of claims 18-21, wherein the pathway of interest is a purine synthesis pathway.

23. The method of any one of claims 18-21, wherein the pathway of interest is a pyrimidine synthesis pathway.

24. The method of any one of claims 18-21, wherein the pathway of interest is a serine synthesis pathway.

25. The method of any one of claims 18-24, further comprising administering the selected therapy to the subject.

26. The method any of claims 18-25, wherein the therapy is an inosine monophosphate dehydrogenase (IMPDH) inhibitor and/or dietary serine restriction.

27. A machine-learning method for estimating absolute activity of one or more metabolic fluxes, the method comprising:
a) administering a tracer to a subject;
b) obtaining a plurality of measurements from samples collected from a subject, wherein each measurement is a level of a metabolite and/or an isotopologue thereof in a metabolic pathway of interest at a single point in time; and
c) applying an artificial intelligence/machine learning (AI/ML) model to the measurements to generate an estimate of absolute activity of one or more metabolic fluxes.

28. The method of claim 27, wherein the pathway of interest is a purine synthesis pathway.

29. The method of claim 28, wherein the one or more metabolites and/or isotopologues thereof comprise one or more of Ribose-5-Phosphate (R5P), Glycine (GLY), Carbon Dioxide (CO2), 5-Methyltetrahydrofolate (C-THF), Inosine Monophosphate (IMP), Inosine, Hypoxanthine, Guanine, Guanosine Monophosphate (GMP), Guanosine Diphosphate (GDP), Guanosine, Adenosine Monophosphate (AMP), and Adenosine.

30. The method of claim 28 or claim 29, further comprising determining a contributing fraction of de novo GMP synthesis and/or de novo IMP synthesis in the purine synthesis pathway.

31. The method of claim 27, wherein the pathway of interest is a pyrimidine synthesis pathway.

32. The method of claim 31, wherein the one or more metabolites and/or isotopologues thereof comprise one or more of Ribose-5-Phosphate (R5P), Aspartate (ASP), Carbon Dioxide (CO2), Uridine, and Uridine Monophosphate (UMP).

33. The method of claim 27, wherein the pathway of interest is a serine synthesis pathway.

34. The method of claim 33, wherein the one or more metabolites and/or isotopologues thereof comprise one or more of 3-phosphoglycerate (3PG), glycine (gly), and 5,10-methylene-THF (Me-THF).

35. The method of any one of claims 27-34, wherein the sample comprises a tissue sample.

36. The method of claim 35, wherein the subject is diagnosed with or at risk of having cancer.

37. The method of claim 36, wherein the cancer is a brain cancer.

38. The method of claim 37, wherein the brain cancer is a glioblastoma.

39. The method of any one of claims 27-38, wherein the tissue sample is a tumor tissue sample.

40. The method of any one of claims 27-39, wherein the tracer comprises a moiety labeled with ¹³C, ¹⁵N, ¹⁸O, or ²D.

41. The method of claim 40, wherein the moiety comprises glucose, methionine, serine, glutamine, lactate, acetate, hypoxanthine, or uridine.

42. The method of any one of claims 36-41, wherein the plurality of measurements are obtained from samples collected after administering radiation therapy to the subject.

43. A method of selecting a therapy for a subject in need thereof, the method comprising:
a) estimating absolute activity of one or more metabolic fluxes by the method of any one of claims 27-42; and
b) selecting therapy for a subject in need thereof based upon the estimated absolute activity of the one or more metabolic fluxes.

44. The method of claim 43, wherein the therapy is an inosine monophosphate dehydrogenase (IMPDH) inhibitor and/or dietary serine restriction.

45. The method of claim 43 or claim 44, further comprising the step of administering the selected therapy to the subject.

46. A method of selecting a therapy for a subject having cancer, the method comprising:
a) determining a contributing fraction of de novo GMP synthesis and/or de novo IMP synthesis in a purine synthesis pathway by the method of claim 5 or claim 30; and
b) selecting a inosine monophosphate dehydrogenase (IMPDH) inhibitor for the subject when the contributing fraction of de novo GMP synthesis and/or de novo IMP synthesis is increased relative to a threshold value.

47. The method of claim 46, wherein the subject has brain cancer.

48. The method of claim 47, wherein the brain cancer is a glioblastoma.

49. A method of selecting a therapy for a subject having cancer, the method comprising:
a) determining absolute activity of a serine synthesis pathway by the method of claim 8, 9, 33, or 34; and
b) selecting dietary serine restriction for the subject when the absolute activity of a serine synthesis pathway is decreased relative to a threshold value.

50. The method of claim 49, wherein the subject has brain cancer.

51. The method of claim 50, wherein the brain cancer is a glioblastoma.
